(19) United States
(12) Patent Application Publication
(10) Pub. No.: US 2026/0367509 A1
(43) Pub. Date: Nov. 19, 2026

(54) RESIST COMPOSITION, RESIST PATTERN FORMATION METHOD, COMPOUND, AND ACID DIFFUSION CONTROL AGENT

(71) Applicant: TOKYO OHKA KOGYO CO., LTD., Kawasaki-shi (JP)

(72) Inventors: Hiroki KATO, Kawasaki-shi (JP); Tetsuya MATSUSHITA, Kawasaki-shi (JP)

(21) Appl. No.: 19/118,533

(22) PCT Filed: Oct. 4, 2023
(86) PCT No.: PCT/JP2023/036248

§ 371 Date: Apr. 4, 2025

(30) Foreign Application Priority Data
Nov. 7, 2022 (JP) 2022-178038

(51) Int. Cl. G03F 7/004; C07C 65/21; C07C 381/12; C07D 333/76; G03F 7/038; G03F 7/039; G03F 7/32

(52) U.S. Cl. CPC G03F 7/0045; C07C 65/21; C07C 381/12; C07D 333/76; G03F 7/0382; G03F 7/0388; G03F 7/0397; G03F 7/32

(57) ABSTRACT

A resist composition in which temporal stability during storage is improved and deterioration of fine resolution after storage is suppressed, a resist pattern formation method using the resist composition, and a compound useful as an acid diffusion control agent used in the resist composition. The resist composition includes a base material component whose solubility in a developing solution is changed by an action of an acid, and a compound represented by General Formula (d0). In Formula (d0), R¹ represents a saturated hydrocarbon group; I represents an iodine atom; R² represents a substituent; Yd⁰ represents a divalent linking group or a single bond; j represents an integer of 0 to 3; k represents an integer of 1 to 4; at least one of Rb¹¹, Rb¹², Rb¹³, or Rb¹⁴ is a fluorine atom or a fluorinated alkyl group; and Rb² and Rb³ represent hydrocarbon groups or are bonded to each other to form a ring together with a sulfur atom

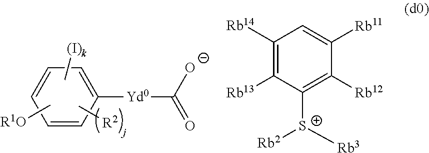

### TECHNICAL FIELD

The present invention relates to a resist composition, a resist pattern formation method, a compound, and an acid diffusion control agent. Priority is claimed on Japanese Patent Application No. 2022-178038, filed Nov. 7, 2022, the content of which is incorporated herein by reference.

### BACKGROUND ART

In recent years, in the manufacture of semiconductor elements and liquid crystal display elements, advances in lithography technologies have led to a rapid progress in the field of pattern fining. These fining methods generally involve shortening the wavelength (increasing the energy) of the exposure light source.

Resist materials are required to have lithography characteristics such as sensitivity to these exposure light sources and resolution that enables reproduction of patterns with fine dimensions.

As a resist material that satisfies these requirements, in the related art, a chemically amplified resist composition containing a base material component whose solubility in a developing solution is changed by an action of an acid and an acid generator component that generates an acid upon light exposure has been used.

In the resist pattern formation, the behavior of the acid generated from the acid generator component upon light exposure is considered as one factor that has a great influence on lithography characteristics.

Meanwhile, a chemically amplified resist composition having both an acid generator component and an acid diffusion control agent that controls the diffusion of an acid generated from the acid generator component upon light exposure has been suggested.

For example, Patent Document 1 discloses a resist composition containing a resin component whose solubility in a developing solution is changed by an action of an acid, an acid generator component, and a photoreactive quencher having a cation moiety of a specific structure, as an acid diffusion control agent. This photoreactive quencher is said to be a component that exhibits a quenching effect by causing an ion exchange reaction with an acid generated from an acid generator component. In a case where such a photoreactive quencher is blended, the diffusion of the acid generated from the acid generator component from exposed portions to unexposed portions of the resist film is controlled, and the lithography characteristics are improved.

### CITATION LIST

### Patent Document

Patent Document 1: Japanese Unexamined Patent Application, First Publication No. 2021-103232

### SUMMARY OF INVENTION

### Technical Problem

However, the resist composition in the related art as described in Patent Document 1 has a problem in that the fine resolution is deteriorated due to storage.

Therefore, an object of the present invention is to provide a resist composition in which temporal stability during storage is improved and deterioration of fine resolution after storage is suppressed, a resist pattern formation method using the resist composition, and a compound useful as an acid diffusion control agent used in the resist composition.

### Solution to Problem

In order to solve the above-described problems, the present invention has adopted the following configurations.

That is, according to a first aspect of the present invention, there is provided a resist composition which generates an acid upon light exposure and whose solubility in a developing solution is changed by an action of the acid, the resist composition containing: a base material component (A) whose solubility in a developing solution is changed by the action of the acid; and a compound (D0) represented by General Formula (d0).

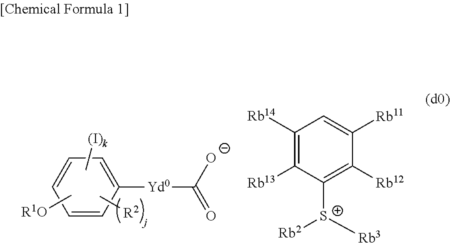

[In the formula, R¹ represents a saturated hydrocarbon group. I represents an iodine atom. R² represents a substituent. Yd⁰ represents a divalent linking group or a single bond. j represents an integer of 0 to 3. k represents an integer of 1 to 4. 1≤j+k≤4 is satisfied. Rb¹¹, Rb¹², Rb¹³, and Rb¹⁴ each independently represent a fluorine atom, a fluorinated alkyl group, or a hydrogen atom. At least one of Rb¹¹, Rb¹², Rb¹³, or Rb¹⁴ is a fluorine atom or a fluorinated alkyl group. Rb² and Rb³ each independently represent a hydrocarbon group which may have a substituent, or Rb² and Rb³ are bonded to each other to form a ring together with a sulfur atom in the formula.]

According to a second aspect of the present invention, there is provided a resist pattern formation method including: a step of forming a resist film on a support using the resist composition according to the first aspect; a step of exposing the resist film to light; and a step of developing the resist film exposed to light to form a resist pattern.

According to a third aspect of the present invention, there is provided a compound which is represented by General Formula (d0).

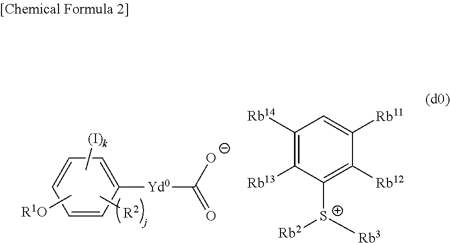

[In the formula, R¹ represents a saturated hydrocarbon group. I represents an iodine atom. R² represents a substituent. Yd⁰ represents a divalent linking group or a single bond. j represents an integer of 0 to 3. k represents an integer of 1 to 4. 1≤j+k≤4 is satisfied. Rb¹¹, Rb¹², Rb¹³, and Rb¹⁴ each independently represent a fluorine atom, a fluorinated alkyl group, or a hydrogen atom. At least one of Rb¹¹, Rb¹², Rb¹³, or Rb¹⁴ is a fluorine atom or a fluorinated alkyl group. Rb² and Rb³ each independently represent a hydrocarbon group which may have a substituent, or Rb² and Rb³ are bonded to each other to form a ring together with a sulfur atom in the formula.]

According to a fourth aspect of the present invention, there is provided an acid diffusion control agent including: the compound according to the third aspect.

### Advantageous Effects of Invention

According to the present invention, it is possible to provide a resist composition in which temporal stability during storage is improved and deterioration of fine resolution after storage is suppressed, a resist pattern formation method using the resist composition, and a compound useful as an acid diffusion control agent used in the resist composition.

### DESCRIPTION OF EMBODIMENTS

In the present specification and the scope of the present claims, the term “aliphatic” is a relative concept used with respect to the term “aromatic” and defines a group, a compound, or the like that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched, or cyclic divalent saturated hydrocarbon group unless otherwise specified.

Examples of “halogen atom” include a fluorine atom, a chlorine atom, a bromine atom, and an iodine atom.

The term “constitutional unit” means a monomer unit constituting a polymer compound (a resin, a polymer, or a copolymer).

The expression “may have a substituent” includes both a case where a hydrogen atom (—H) is substituted with a monovalent group and a case where a methylene group (—CH₂—) is substituted with a divalent group.

The term “light exposure” is a general concept for irradiation with radiation.

The term “acid decomposable group” is a group having acid decomposability in which at least a part of bond in the structure of the acid decomposable group can be cleaved by an action of an acid.

Examples of the acid decomposable group whose polarity is increased by the action of an acid include groups which are decomposed by the action of an acid to generate a polar group.

Examples of the polar group include a carboxy group, a hydroxyl group, an amino group, and a sulfo group (—SO₃H).

More specific examples of the acid decomposable group include a group in which the above-described polar group has been protected by an acid dissociable group (for example, a group in which a hydrogen atom of the OH-containing polar group has been protected by an acid dissociable group).

Here, the term “acid dissociable group” indicates both a group (i) having an acid dissociation property in which a bond between the acid dissociable group and an atom adjacent to the acid dissociable group can be cleaved by the action of an acid and a group (ii) in which some bonds are cleaved by the action of an acid, a decarboxylation reaction further occurs, and thus the bond between the acid dissociable group and the atom adjacent to the acid dissociable group can be cleaved.

It is necessary that the acid dissociable group that constitutes the acid decomposable group is a group which exhibits a lower polarity than that of the polar group generated by the dissociation of the acid dissociable group. Thus, in a case where the acid dissociable group is dissociated by the action of an acid, a polar group exhibiting a higher polarity than that of the acid dissociable group is generated so that the polarity is increased. As a result, the polarity of an entire component (A1) is increased. Due to the increase in the polarity, the solubility in a developing solution is relatively changed such that the solubility is increased in a case where the developing solution is an alkali developing solution and the solubility is decreased in a case where the developing solution is an organic developing solution.

The term “base material component” is an organic compound having a film-forming ability. The organic compounds used as the base material component are classified into non-polymers and polymers. As the non-polymers, typically non-polymers having a molecular weight of 500 or greater and less than 4000 (hereinafter, referred to as “low-molecular-weight compounds”) are used. Hereinafter, “resin”, “polymer compound”, or “polymer” indicates a polymer having a molecular weight of 1000 or greater. As the molecular weight of the polymer, the weight-average molecular weight in terms of polystyrene determined by gel permeation chromatography (GPC) is used.

The expression “constitutional unit derived from” means a constitutional unit that is formed by the cleavage of a multiple bond between carbon atoms, for example, an ethylenic double bond.

In “acrylic acid ester”, the hydrogen atom bonded to the carbon atom at the α-position may be substituted with a substituent. The substituent (R^{αx}) that substitutes the hydrogen atom bonded to the carbon atom at the α-position is an atom other than the hydrogen atom or a group. Further, the acrylic acid ester includes itaconic acid diester in which the substituent (R^{αx}) has been substituted with a substituent having an ester bond or α-hydroxyacryl ester in which the substituent (R^{αx}) has been substituted with a hydroxyalkyl group or a group obtained by modifying a hydroxyl group thereof. Further, the carbon atom at the α-position of acrylic acid ester indicates the carbon atom to which the carbonyl group of acrylic acid is bonded unless otherwise specified.

Hereinafter, acrylic acid ester in which the hydrogen atom bonded to the carbon atom at the α-position has been substituted with a substituent is also referred to as an α-substituted acrylic acid ester.

The term “derivative” is used as a concept that includes those obtained by substituting a hydrogen atom at the α-position of a target compound with another substituent such as an alkyl group or a halogenated alkyl group, and a derivative thereof. Examples of the derivative thereof include a derivative obtained by substituting a hydrogen atom of a hydroxyl group of a target compound, in which the hydrogen atom at the α-position may be substituted with a substituent, with an organic group; and a derivative obtained by bonding a substituent other than a hydroxyl group to a target compound in which the hydrogen atom at the α-position may be substituted with a substituent. The α-position refers to the first carbon atom adjacent to the functional group unless otherwise specified.

Examples of the substituent that substitutes the hydrogen atom at the α-position of hydroxystyrene include the same substituent as those for R^{αx}.

In the present specification and the scope of the present claims, asymmetric carbons may be present and enantiomers or diastereomers may be present depending on the structures represented by the chemical formulae. In this case, these isomers are represented by one chemical formula. These isomers may be used alone or used as a mixture.

### (Resist Composition)

The resist composition of the present embodiment is a resist composition which generates an acid upon light exposure and whose solubility in a developing solution is changed by the action of the acid.

Such a resist composition contains a base material component (A) (hereinafter, also referred to as “component (A)”) whose solubility in a developing solution is changed by the action of the acid, and a compound (D0) (hereinafter, also referred to as “component (D0)”) represented by General Formula (d0) described below.

In the resist composition of the present embodiment, the component (A) may generate an acid upon light exposure, or an additive component that is blended separately from the component (A) may generate an acid upon light exposure.

Specifically, the resist composition of the present embodiment may (1) further contain an acid generator component (B) (hereinafter, referred to as “component (B)”) that generates an acid upon light exposure; (2) the component (A) may be a component that generates an acid upon light exposure; and (3) the component (A) may be a component that generates an acid upon light exposure and further contain a component (B).

That is, in the cases of (2) and (3) described above, the component (A) is “base material component which generates an acid upon light exposure and whose solubility in a developing solution is changed by the action of the acid”. In a case where the component (A) is a base material component which generates an acid upon light exposure and whose solubility in a developing solution is changed by the action of the acid, it is preferable that the component (A1) described below is a resin which generates an acid upon light exposure and whose solubility in a developing solution is changed by the action of the acid. As such a resin, a polymer compound having a constitutional unit that generates an acid upon light exposure can be used. As the constitutional unit that generates an acid upon light exposure, known constitutional units can be used.

Among these, it is preferable that the resist composition of the present embodiment corresponds to the case (1). That is, it is preferable that the resist composition of the present embodiment contains the component (A) and the component (B).

In a case where a resist film is formed using the resist composition of the present embodiment and the resist film is subjected to the selective light exposure, for example, since an acid is generated from the component (B) and the solubility of the component (A) in a developing solution is changed by the action of the acid in an exposed portion of the resist film while the solubility of the component (A) in a developing solution is not changed in an unexposed portion of the resist film, a difference in solubility in the developing solution occurs between the exposed portion and the unexposed portion. Therefore, in a case where the resist film is developed, the exposed portion of the resist film is dissolved and removed to form a positive-tone resist pattern in a case where the resist composition is a positive-tone type, whereas the unexposed portion of the resist film is dissolved and removed to form a negative-tone resist pattern in a case where the resist composition is a negative-tone type.

The resist composition of the present embodiment may be a positive-tone resist composition or a negative-tone resist composition. Further, in the formation of a resist pattern, the resist composition of the present embodiment may be applied to an alkali developing process using an alkali developing solution in the developing treatment, or a solvent developing process using a developing solution containing an organic solvent (organic developing solution) in the developing treatment.

### <Component (A)>

In the resist composition of the present embodiment, it is preferable that the component (A) contains a resin component (A1) whose solubility in a developing solution is changed by the action of an acid (hereinafter, also referred to as “component (A1)”).

Since the polarity of the base material component before and after the light exposure is changed by using the component (A1), an excellent development contrast can be obtained not only in the alkali developing process but also in the solvent developing process.

As the component (A), another polymer compound and/or a low-molecular-weight compound may be used in combination with the component (A1).

In the resist composition of the present embodiment, the component (A) may be used alone or in combination of two or more kinds thereof.

### In Regard to Component (A1)

The component (A1) is a resin component whose solubility in a developing solution is changed by the action of an acid.

It is preferable that the component (A1) has a constitutional unit (al) containing an acid decomposable group whose polarity is increased by the action of an acid.

The component (A1) may have other constitutional units as necessary in addition to the constitutional unit (a1).

<<Constitutional unit (a1)>>

The constitutional unit (a1) is a constitutional unit that contains an acid decomposable group whose polarity is increased by the action of an acid.

Examples of the acid dissociable group include the same as those which have been suggested so far as the acid dissociable groups of the base resin for a chemically amplified resist composition.

Specific examples of the suggested acid dissociable group of the base resin for a chemically amplified resist composition include “acetal type acid dissociable group”, “tertiary alkyl ester type acid dissociable group”, and “tertiary alkyloxycarbonyl acid dissociable group” described below.

Acetal type acid dissociable group:

Examples of the acid dissociable group that protects a carboxy group or a hydroxyl group in the polar groups include an acid dissociable group represented by General Formula (a1-r-1) (hereinafter, also referred to as “acetal type acid dissociable group”).

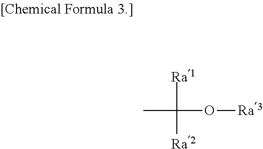

[In the formula, Ra′¹ and Ra′² represent a hydrogen atom or an alkyl group. Ra′³ represents a hydrocarbon group, and Ra′³ may be bonded to any of Ra′¹ and Ra′² to form a ring.]

In Formula (a1-r-1), it is preferable that at least one of Ra′¹ and Ra′² represents a hydrogen atom and more preferable that both Ra′¹ and Ra′² represent a hydrogen atom.

In a case where Ra′¹ or Ra′² represents an alkyl group, examples of the alkyl group include the same alkyl groups described as the substituent which may be bonded to the carbon atom at the α-position in the description on α-substituted acrylic acid ester. Among these, an alkyl group having 1 to 5 carbon atoms is preferable. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′³ represents a cyclic hydrocarbon group, the hydrocarbon group may be an alicyclic hydrocarbon group or an aromatic hydrocarbon group and may be a polycyclic group or a monocyclic group.

As the alicyclic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the alicyclic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclo[5.2.1.0^{2,6}]decane, and tetracyclododecane.

In a case where the cyclic hydrocarbon group as Ra′³ is an aromatic hydrocarbon group, the aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms.

Specific examples of the aromatic ring include an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (for example, biphenyl or fluorene); and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The alkylene group bonded to the aromatic hydrocarbon ring or aromatic heterocyclic ring has preferably 1 to 4 carbon atoms, more preferably 1 or 2 carbon atoms, and particularly preferably 1 carbon atom.

The cyclic hydrocarbon group as Ra′³ may have a substituent. Examples of the substituent include —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN, and —R^{P2}—COOH) (hereinafter, these substituents will also be collectively referred to as “R^{αx5}”).

Here, R^{P1} represents a monovalent chain-like saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. Further, R^{P2} represents a single bond, a divalent chain-like saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. Some or all hydrogen atoms in the chain-like saturated hydrocarbon group, the aliphatic cyclic saturated hydrocarbon group, and the aromatic hydrocarbon group as R^{P1} and R^{P2} may be substituted with fluorine atoms. The aliphatic cyclic hydrocarbon group may have one or more of a single kind of the substituents or one or more of each of plurality of kinds of the substituents.

Examples of the monovalent chain-like saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.0^{2,6}]decanyl group, a tricyclo[3.3.1.13,7]decanyl group, a tetracyclo[6.2.1.13,6.02,7]dodecanyl group, or an adamantyl group.

Examples of the monovalent aromatic hydrocarbon group having 6 to 30 carbon atoms include a group in which one hydrogen atom has been removed from an aromatic hydrocarbon ring such as benzene, biphenyl, fluorene, naphthalene, anthracene, or phenanthrene.

In a case where Ra′³ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

Tertiary alkyl ester type acid dissociable group:

Examples of the acid dissociable group that protects a carboxy group among the polar groups include an acid dissociable group represented by General Formula (a1-r-2).

Among the examples of the acid dissociable group represented by Formula (a1-r-2), a group formed of an alkyl group may be referred to as “tertiary alkyl ester type acid dissociable group” for convenience.

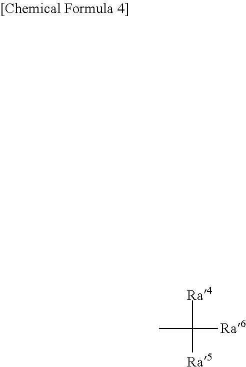

[In the formula, Ra′⁴ to Ra′⁶ each represent a hydrocarbon group, and Ra′⁵ and Ra′⁶ may be bonded to each other to form a ring.]

Examples of the hydrocarbon group as Ra′⁴ include a linear or branched alkyl group, a chain-like or cyclic alkenyl group, a chain-like alkynyl group, and a cyclic hydrocarbon group.

Examples of the linear or branched alkyl group and the cyclic hydrocarbon group (an alicyclic hydrocarbon group which is a monocyclic group, an alicyclic hydrocarbon group which is a polycyclic group, or an aromatic hydrocarbon group) as Ra′⁴ include the same groups as those for Ra′³.

As the chain-like or cyclic alkenyl group as Ra′⁴, an alkenyl group having 2 to 10 carbon atoms is preferable.

Examples of the hydrocarbon group as Ra′⁵ or Ra′⁶ include the same groups as those for Ra′³.

In a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

Meanwhile, in a case where Ra′⁴ to Ra′⁶ represent an independent hydrocarbon group without being bonded to one another, suitable examples thereof include a group represented by General Formula (a1-r2-4).

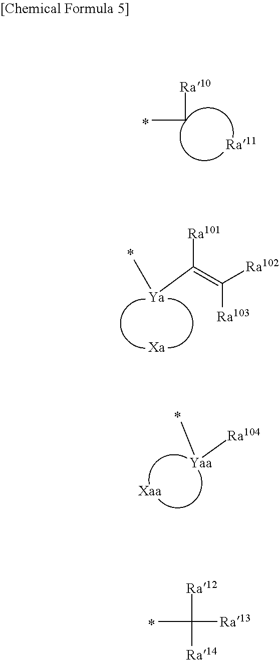

[In Formula (a1-r2-1), Ra′¹⁰ represents a linear or branched alkyl group having 1 to 12 carbon atoms, in which a part thereof may be substituted with a halogen atom or a heteroatom-containing group. Ra′¹¹ represents a group that forms an aliphatic cyclic group together with the carbon atom to which Ra′¹⁰ is bonded. In Formula (a1-r2-2), Ya represents a carbon atom. Xa represents a group that forms a cyclic hydrocarbon group together with Ya. Some or all hydrogen atoms in the cyclic hydrocarbon group may be substituted. Ra¹⁰¹ to Ra¹⁰³ each independently represent a hydrogen atom, a monovalent chain-like saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra¹⁰¹ to Ra¹⁰³ may be bonded to one another to form a cyclic structure. In Formula (a1-r2-3), Yaa represents a carbon atom. Xaa represents a group that forms an aliphatic cyclic group together with Yaa. Ra¹⁰⁴ represents an aromatic hydrocarbon group which may have a substituent. In Formula (a1-r2-4), Ra′¹² and Ra′¹³ each independently represent a monovalent chain-like saturated hydrocarbon group having 1 to 10 carbon atoms. Some or all hydrogen atoms in the chain-like saturated hydrocarbon group may be substituted. Ra′¹⁴ represents a hydrocarbon group which may have a substituent. * represents a bonding site (the same applies hereinafter).]

In Formula (a1-r2-1), Ra′¹⁰ represents a linear or branched alkyl group having 1 to 12 carbon atoms, in which a part thereof may be substituted with a halogen atom or a heteroatom-containing group.

The linear alkyl group as Ra′¹⁰ has 1 to 12 carbon atoms, preferably 1 to 10 carbon atoms, and particularly preferably 1 to 5 carbon atoms.

Examples of the branched alkyl group as Ra′¹⁰ include the same groups as those for Ra′³.

A part of the alkyl group as Ra′¹⁰ may be substituted with a halogen atom or a heteroatom-containing group. For example, some hydrogen atoms constituting the alkyl group may be substituted with a halogen atom or a heteroatom-containing group. Further, some carbon atoms (such as a methylene group) constituting the alkyl group may be substituted with a heteroatom-containing group.

Examples of the heteroatoms here include an oxygen atom, a nitrogen atom, and a sulfur atom. Examples of the heteroatom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

In Formula (a1-r2-1), preferred examples of Ra′¹¹ (an aliphatic cyclic group that is formed together with a carbon atom to which Ra′¹⁰ is bonded) include the groups described as the alicyclic hydrocarbon group (alicyclic hydrocarbon group) which is a monocyclic group or a polycyclic group as Ra′³ in Formula (a1-r-1). Among these, a monocyclic alicyclic hydrocarbon group is preferable, and specifically, a cyclopentyl group or a cyclohexyl group is more preferable.

In Formula (a1-r2-2), examples of the cyclic hydrocarbon group that is formed by Xa together with Ya include a group in which one or more hydrogen atoms have been further removed from the cyclic monovalent hydrocarbon group (alicyclic hydrocarbon group) as Ra′³ in Formula (a1-r-1).

The cyclic hydrocarbon group that is formed by Xa together with Ya may have a substituent. Examples of the substituent include those which are the same as the substituents which may be included in the cyclic hydrocarbon group as Ra′³.

In Formula (a1-r2-2), examples of the monovalent chain-like saturated hydrocarbon group having 1 to 10 carbon atoms as Ra¹⁰¹ to Ra¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.0^{2,6}]decanyl group, a tricyclo[3.3.1.13,7]decanyl group, a tetracyclo[6.2.1.13,6.02,7]dodecanyl group, or an adamantyl group.

From the viewpoint of ease of synthesis, Ra¹⁰¹ to Ra¹⁰³ are preferably a hydrogen atom or a monovalent chain-like saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ include the same groups as those for R^{αx5} described above.

Examples of the group having a carbon-carbon double bond generated by two or more of Ra¹⁰¹ to Ra¹⁰³ being bonded to one another to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint of ease of synthesis, a cyclopentenyl group, a cyclohexenyl group, or a cyclopentylidenethenyl group is preferable.

In Formula (a1-r2-3), as the aliphatic cyclic group that is formed by Xaa together with Yaa, the group described as the alicyclic hydrocarbon group which is a monocyclic group or a polycyclic group as Ra′³ in Formula (a1-r-1) is preferable.

In Formula (a1-r2-3), examples of the aromatic hydrocarbon group as Ra¹⁰⁴ include a group in which one or more hydrogen atoms have been removed from an aromatic hydrocarbon ring having 5 to 30 carbon atoms. Among these, Ra¹⁰⁴ is preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent which may be included in Ra¹⁰⁴ in Formula (a1-r2-3) include a methyl group, an ethyl group, a propyl group, a hydroxyl group, a carboxyl group, a halogen atom, an alkoxy group (such as a methoxy group, an ethoxy group, a propoxy group, or a butoxy group), and an alkyloxycarbonyl group.

In Formula (a1-r2-4), Ra′¹² and Ra′¹³ each independently represent a monovalent chain-like saturated hydrocarbon group having 1 to 10 carbon atoms. Examples of the monovalent chain-like saturated hydrocarbon group having 1 to 10 carbon atoms as Ra′¹² and Ra′¹³ include the same one as the monovalent chain-like saturated hydrocarbon group having 1 to 10 carbon atoms as Ra¹⁰¹ to Ra¹⁰³ described above. Some or all hydrogen atoms in the chain-like saturated hydrocarbon group may be substituted.

Among these, Ra′¹² and Ra′¹³ are preferably an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon group represented by Ra′¹² and Ra′¹³ is substituted, examples of the substituent are the same groups as those for R^{αx5} described above.

In Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an alicyclic hydrocarbon group or an aromatic hydrocarbon group and may be a polycyclic group or a monocyclic group.

As the alicyclic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the alicyclic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclo[5.2.1.0^{2,6}]decane, and tetracyclododecane.

Examples of the aromatic hydrocarbon group as Ra′¹⁴ include the same groups as those for the aromatic hydrocarbon group as Ra¹⁰⁴. Among these, Ra′¹⁴ is preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent which may be included in Ra′¹⁴ include those which are the same as the substituent which may be included in Ra¹⁰⁴.

In a case where Ra′¹⁴ in Formula (a1-r2-4) represents a naphthyl group, the position bonded to the tertiary carbon atom in Formula (a1-r2-4) may be any of the 1-position or the 2-position of the naphthyl group.

In a case where Ra′¹⁴ in Formula (a1-r2-4) represents an anthryl group, the position bonded to the tertiary carbon atom in Formula (a1-r2-4) may be any of the 1-position, the 2-position, or the 9-position of the anthryl group.

Specific examples of the group represented by Formula (a1-r2-1) are shown below.

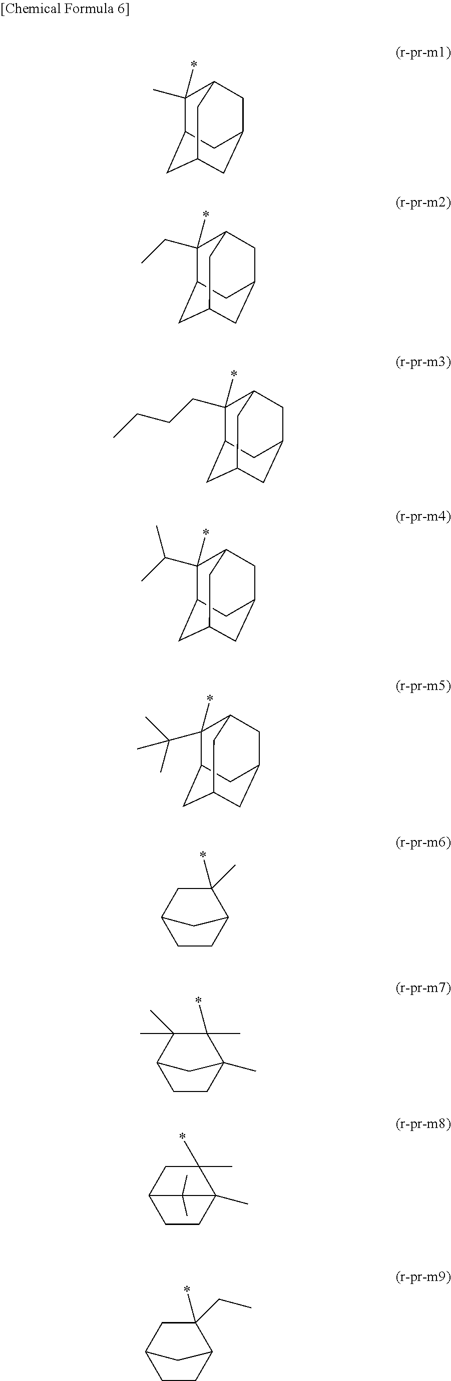

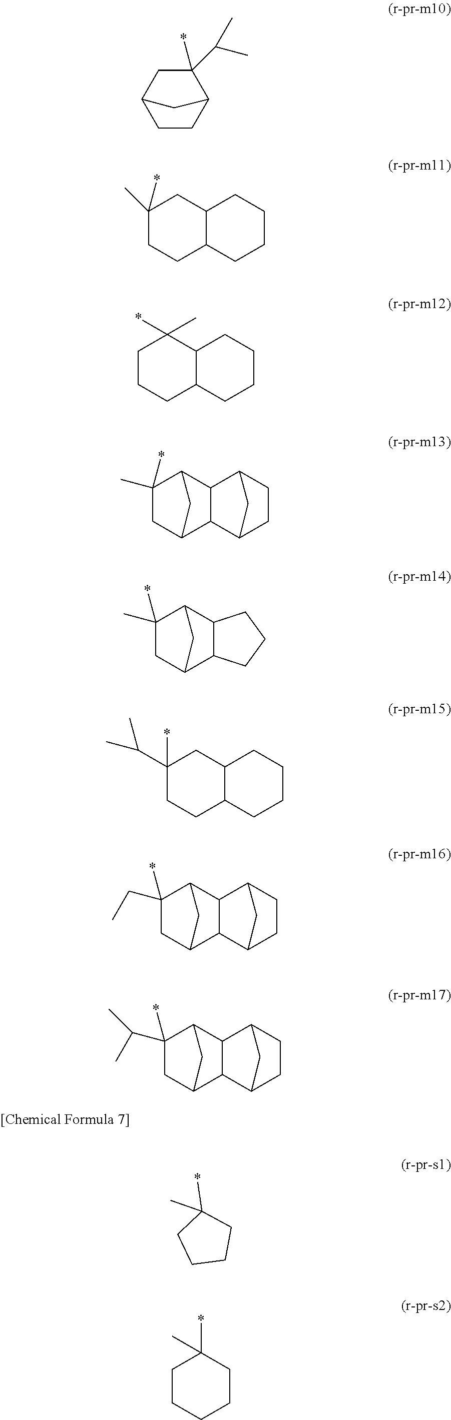

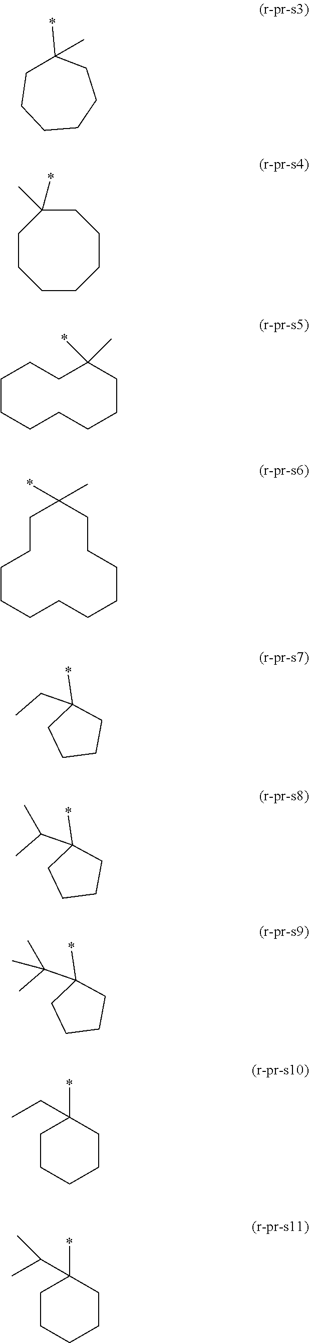

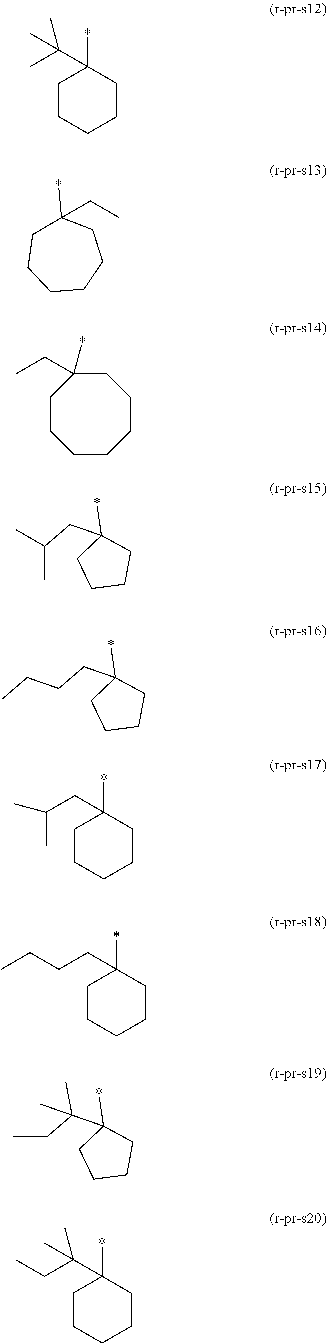

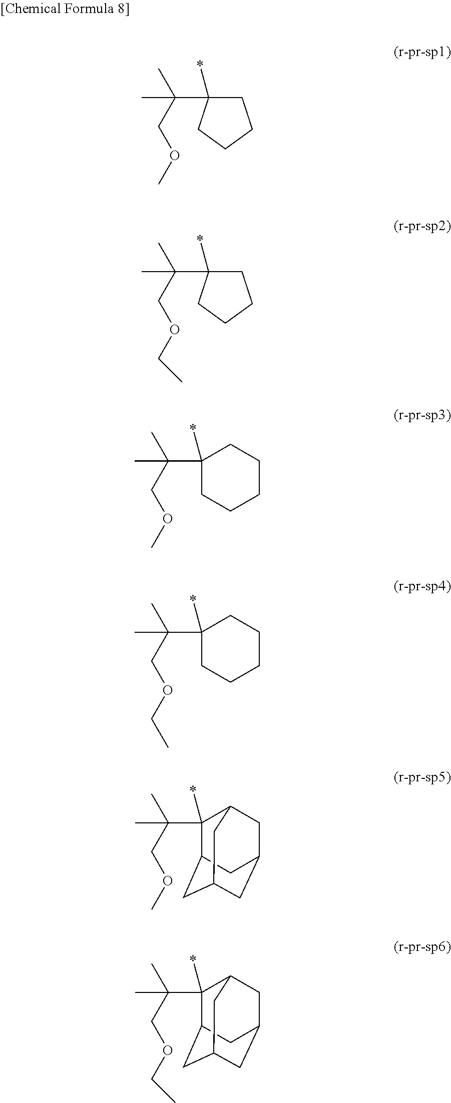 [SMILES: *C1(C(C)(C)COC)CCCC1 ; *C1(C(C)(C)COCC)CCCC1 ; *C1(C(C)(C)COC)CCCCC1 ; *C1(C(C)(C)COCC)CCCCC1 ; *C1(C(C)(C)COC)C2CC3CC(C2)CC1C3 ; *C1(C(C)(C)COCC)C2CC3CC(C2)CC1C3]

Specific examples of the group represented by Formula (a1-r2-2) are shown below.

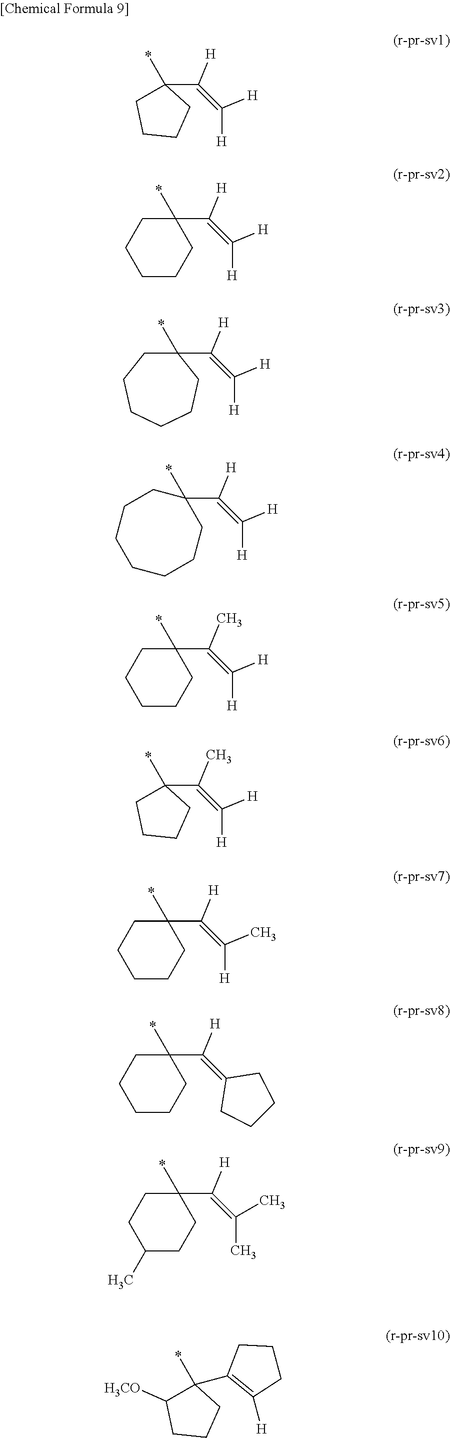

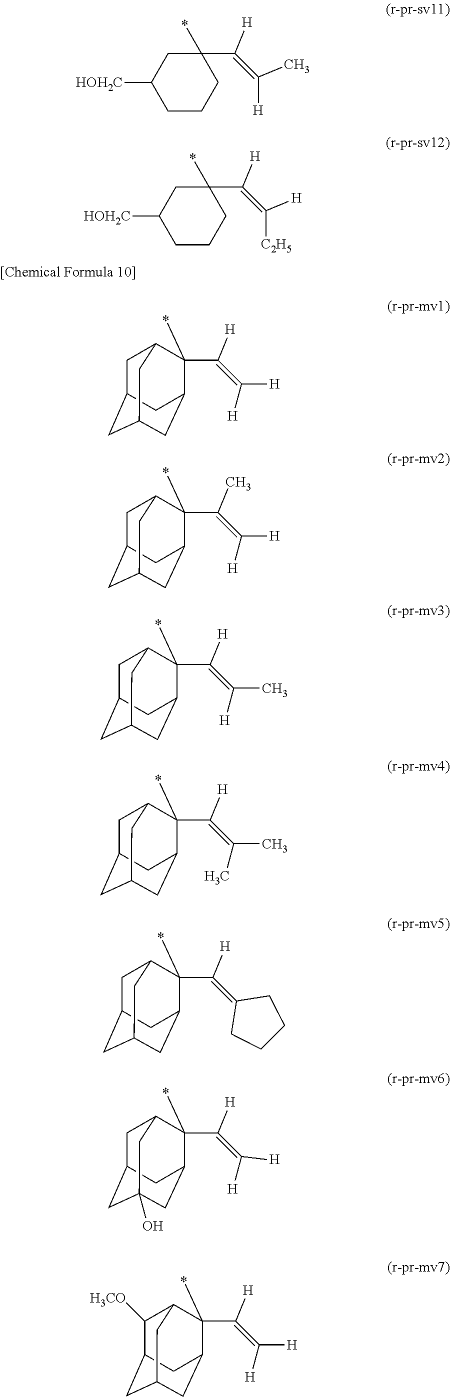

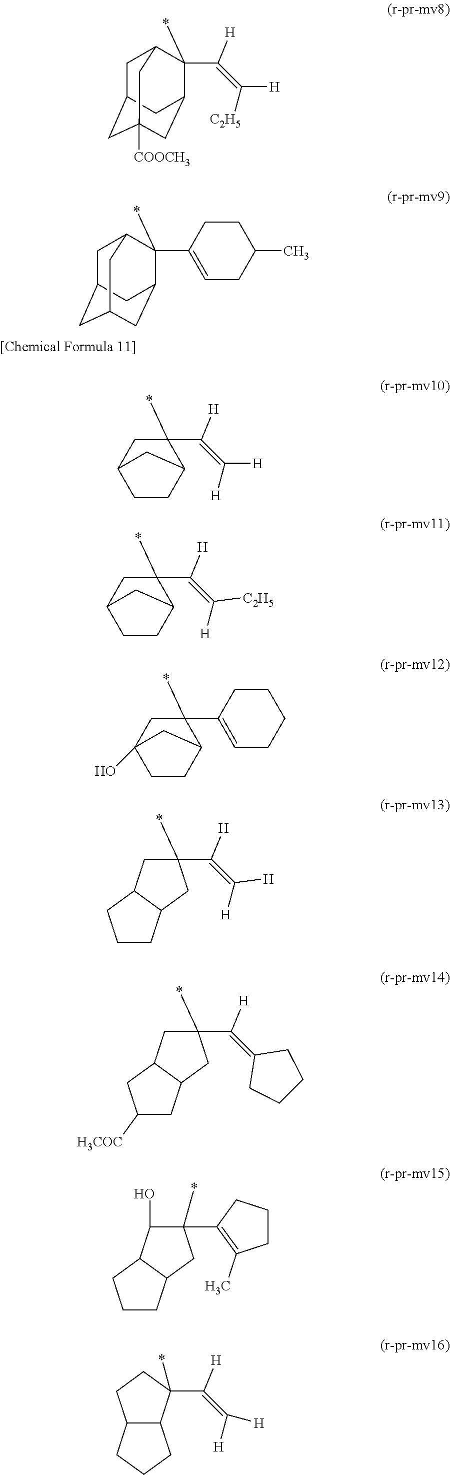

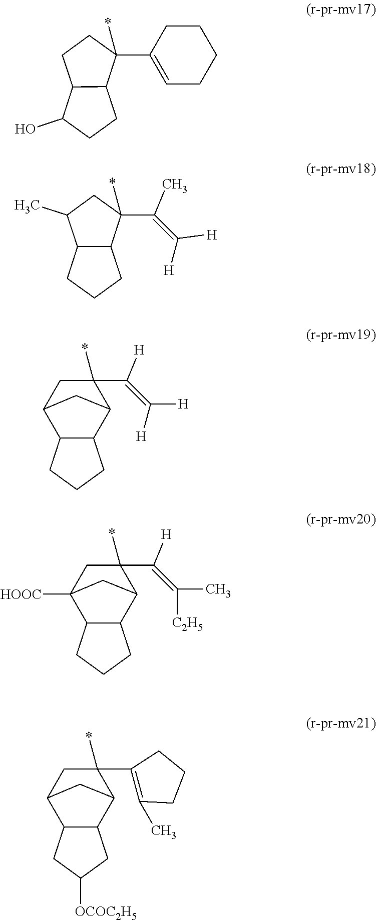

Specific examples of the group represented by Formula (a1-r2-3) are shown below.

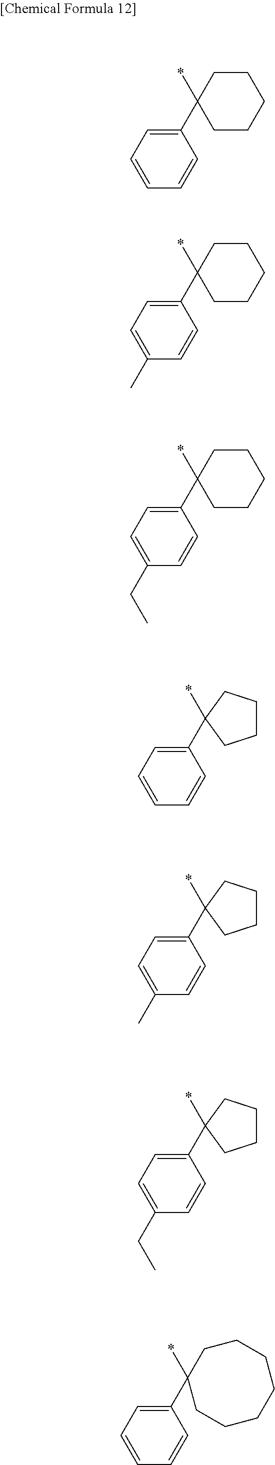

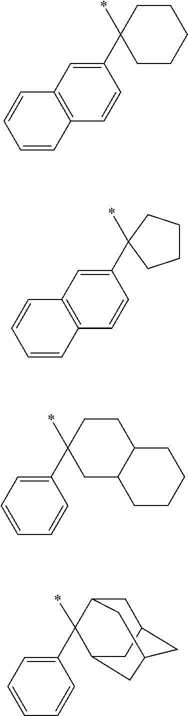 [SMILES: *C1(c2ccc3ccccc3c2)CCCCC1 ; *C1(c2ccc3ccccc3c2)CCCC1 ; *C1(c2ccccc2)CCC2CCCCC2C1 ; *C1(c2ccccc2)C2CC3CC(C2)CC1C3]

Specific examples of the group represented by Formula (a1-r2-4) are shown below.

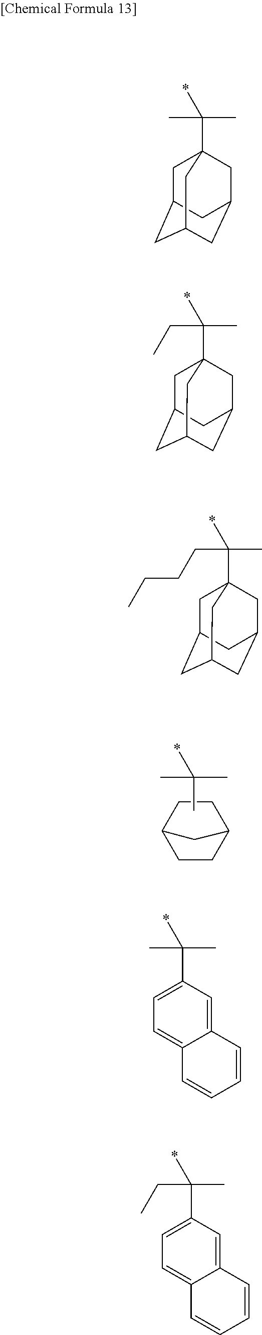

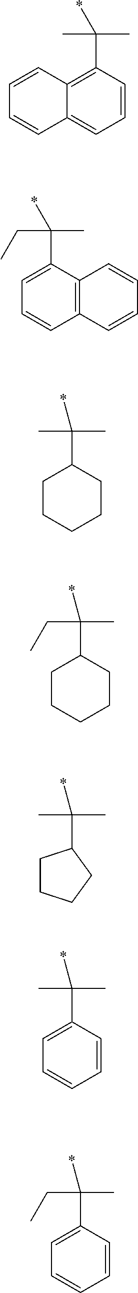

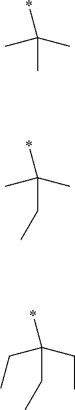

Tertiary alkyloxycarbonyl acid dissociable group:

Examples of the acid dissociable group that protects a hydroxyl group among the polar groups include an acid dissociable group (hereinafter, also referred to as “tertiary alkyloxycarbonyl acid dissociable group” for convenience) represented by General Formula (a1-r-3).

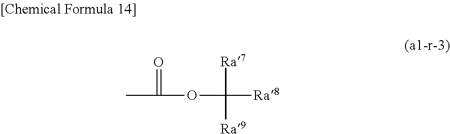

[In the formula, Ra′⁷ to Ra′⁹ each represent an alkyl group.]

In Formula (a1-r-3), Ra′⁷ to Ra′⁹ are each preferably an alkyl group having 1 to 5 carbon atoms and more preferably an alkyl group having 1 to 3 carbon atoms.

Further, the total number of carbon atoms in each alkyl group is preferably 3 to 7, more preferably 3 to 5, and most preferably 3 or 4.

Secondary alkyl ester type acid dissociable group:

Examples of the acid dissociable group that protects a carboxy group among the polar groups include an acid dissociable group represented by General Formula (a1-r-4).

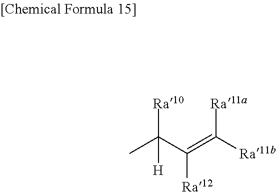

[In the formula, Ra′¹⁰ represents a hydrocarbon group. Ra′^{11a} and Ra′^{11b} each independently represent a hydrogen atom, a halogen atom, or an alkyl group. Ra′¹² represents a hydrogen atom or a hydrocarbon group. Ra′¹⁰ and Ra′^{11a} or Ra′^{11b} may be bonded to each other to form a ring. Ra′^{11a} or Ra′^{11b} and Ra′¹² may be bonded to each other to form a ring.]

Examples of the hydrocarbon group as Ra′¹⁰ and Ra′¹² in the formula include the same groups as those for Ra′³.

Examples of the alkyl group as Ra′^{11a} and Ra′^{11b} in the formula include the same groups as those for the alkyl group as Ra′¹.

In the formula, the hydrocarbon group as Ra′¹⁰ and Ra′¹² and the alkyl group as Ra′^{11a} and Ra′^{11b} may have a substituent. Examples of this substituent include R^{αx5} described above.

Ra′¹⁰ and Ra′^{11a} or Ra′^{11b} may be bonded to each other to form a ring. The ring may be a polycyclic ring or a monocyclic ring, and may be an alicyclic ring or an aromatic ring.

The alicyclic ring and the aromatic ring may have a heteroatom.

Among these, as the ring formed by Ra′¹⁰ and Ra′^{11a} or Ra′^{11b} being bonded to each other, monocycloalkene, a ring in which some carbon atoms of monocycloalkene are substituted with heteroatoms (such as an oxygen atom and a sulfur atom), or monocycloalkadiene is preferable, cycloalkene having 3 to 6 carbon atoms is preferable, and cyclopentene or cyclohexene is preferable.

The ring formed by Ra′¹⁰ and Ra′^{11a} or Ra′^{11b} being bonded to each other may be a condensed ring. Specific examples of the condensed ring include indane.

The ring formed by Ra′¹⁰ and Ra′^{11a} or Ra′^{11b} being bonded to each other may have a substituent. Examples of this substituent include R^{αx5} described above.

Ra′^{11a} or Ra′^{11b} and Ra′¹² may be bonded to each other to form a ring, and examples of the ring include the same rings as the rings formed by Ra′¹⁰ and Ra′^{11a} or Ra′^{11b} being bonded to each other.

Specific examples of the group represented by Formula (a1-r-4) are shown below.

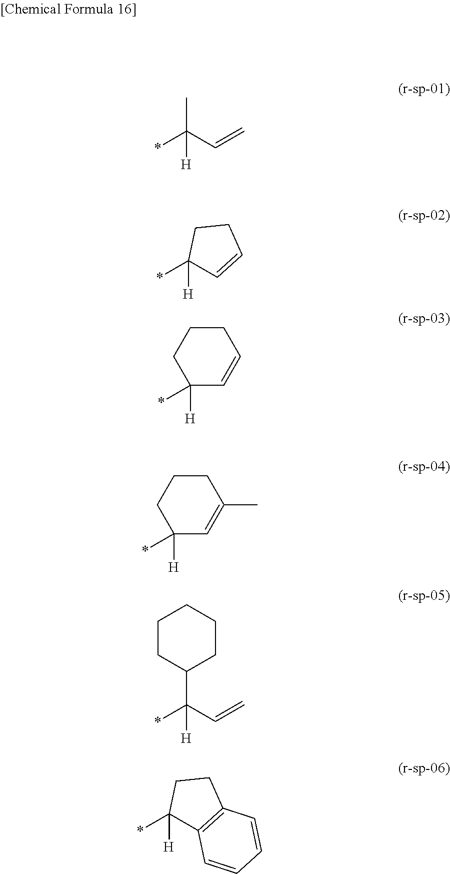

Examples of the constitutional unit (a1) include a constitutional unit derived from acrylic acid ester in which the hydrogen atom bonded to the carbon atom at the α-position may be substituted with a substituent, a constitutional unit derived from acrylamide, a constitutional unit in which at least some hydrogen atoms in a hydroxyl group of a constitutional unit derived from hydroxystyrene or a hydroxystyrene derivative are protected by a substituent containing the acid decomposable group, and a constitutional unit in which at least some hydrogen atoms in —C(—O)—OH of a constitutional unit derived from vinylbenzoic acid or a vinylbenzoic acid derivative are protected by a substituent containing the acid decomposable group.

Among these, as the constitutional unit (a1), a constitutional unit derived from acrylic acid ester in which the hydrogen atom bonded to the carbon atom at the α-position may be substituted with a substituent is preferable.

Specific preferred examples of such a constitutional unit (a1) include constitutional units represented by General Formula (a1-1) or (a1-2).

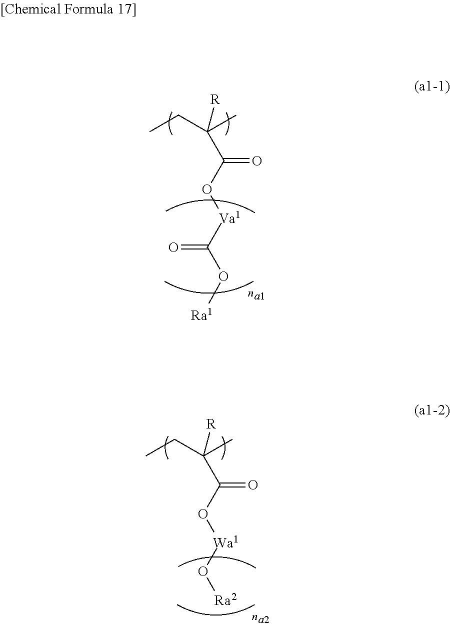

[In the formula, R represents a hydrogen atom, an alkyl group having 1 to 5 carbon atoms, or a halogenated alkyl group having 1 to 5 carbon atoms. Va¹ represents a divalent hydrocarbon group which may have an ether bond. nₐ₁ represents an integer of 0 to 2. Ral represents an acid dissociable group represented by General Formula (a1-r-1), (a1-r-2), or (a1-r-4). Wa¹ represents an (nₐ₂+1)-valent hydrocarbon group, nₐ₂ represents an integer of 1 to 3, and Ra² represents an acid dissociable group represented by General Formula (a1-r-1) or (a1-r-3).]

In Formula (a1-1), as the alkyl group having 1 to 5 carbon atoms as R, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R represents preferably a hydrogen atom, an alkyl group having 1 to 5 carbon atoms, or a fluorinated alkyl group having 1 to 5 carbon atoms, and from the viewpoint of industrial availability, most preferably a hydrogen atom or a methyl group.

In Formula (a1-1), the divalent hydrocarbon group as Va¹ may be an aliphatic hydrocarbon group or an aromatic hydrocarbon group.

The aliphatic hydrocarbon group as the divalent hydrocarbon group represented by Va¹ may be saturated or unsaturated. In general, it is preferable that the aliphatic hydrocarbon group is saturated.

More specific examples of the aliphatic hydrocarbon group include a linear or branched aliphatic hydrocarbon group and an aliphatic hydrocarbon group having a ring in the structure thereof.

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable. Specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Examples of the aliphatic hydrocarbon group having a ring in the structure thereof include an alicyclic hydrocarbon group (a group in which two hydrogen atoms have been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group. Examples of the linear or branched aliphatic hydrocarbon group include the same groups as those for the linear aliphatic hydrocarbon group or the branched aliphatic hydrocarbon group.

The alicyclic hydrocarbon group has preferably 3 to 20 carbon atoms and more preferably 3 to 12 carbon atoms.

The alicyclic hydrocarbon group may be polycyclic or monocyclic. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable, and specific examples thereof include adamantane, norbornane, isobornane, tricyclo[5.2.1.0^{2,6}]decane, and tetracyclododecane.

The aromatic hydrocarbon group as the divalent hydrocarbon group represented by Va¹ is a hydrocarbon group having an aromatic ring.

Such an aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 12 carbon atoms. Here, the number of carbon atoms in a substituent is not included in the number of carbon atoms.

Specific examples of the aromatic ring contained in the aromatic hydrocarbon group include aromatic hydrocarbon rings such as benzene, biphenyl, fluorene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom.

Specific examples of the aromatic hydrocarbon group include a group in which two hydrogen atoms have been removed from the aromatic hydrocarbon ring (an arylene group); and a group in which one hydrogen atom of a group (an aryl group) obtained by removing one hydrogen atom from the aromatic hydrocarbon ring has been substituted with an alkylene group (for example, a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The alkylene group (an alkyl chain in the arylalkyl group) has preferably 1 to 4 carbon atoms, more preferably 1 or 2 carbon atoms, and particularly preferably 1 carbon atom.

In Formula (a1-1), Ra¹ represents an acid dissociable group represented by Formula (a1-r-1), (a1-r-2), or (a1-r-4).

In Formula (a1-2), the (nₐ₂+1)-valent hydrocarbon group as Wa¹ may be an aliphatic hydrocarbon group or an aromatic hydrocarbon group. The aliphatic hydrocarbon group means a hydrocarbon group that has no aromaticity and may be saturated or unsaturated. In general, it is preferable that the aliphatic hydrocarbon group is saturated. Examples of the aliphatic hydrocarbon group include a linear or branched aliphatic hydrocarbon group, an aliphatic hydrocarbon group having a ring in the structure thereof, and a group obtained by combining the linear or branched aliphatic hydrocarbon group and the aliphatic hydrocarbon group having a ring in the structure thereof.

The valency of nₐ₂+1 is preferably divalent, trivalent, or tetravalent, and more preferably divalent or trivalent.

In Formula (a1-2), Ra² represents an acid dissociable group represented by General Formula (a1-r-1) or (a1-r-3).

Specific examples of the constitutional unit (a1) are shown below. In the formulae shown below, R^{α} represents a hydrogen atom, a methyl group, or a trifluoromethyl group.

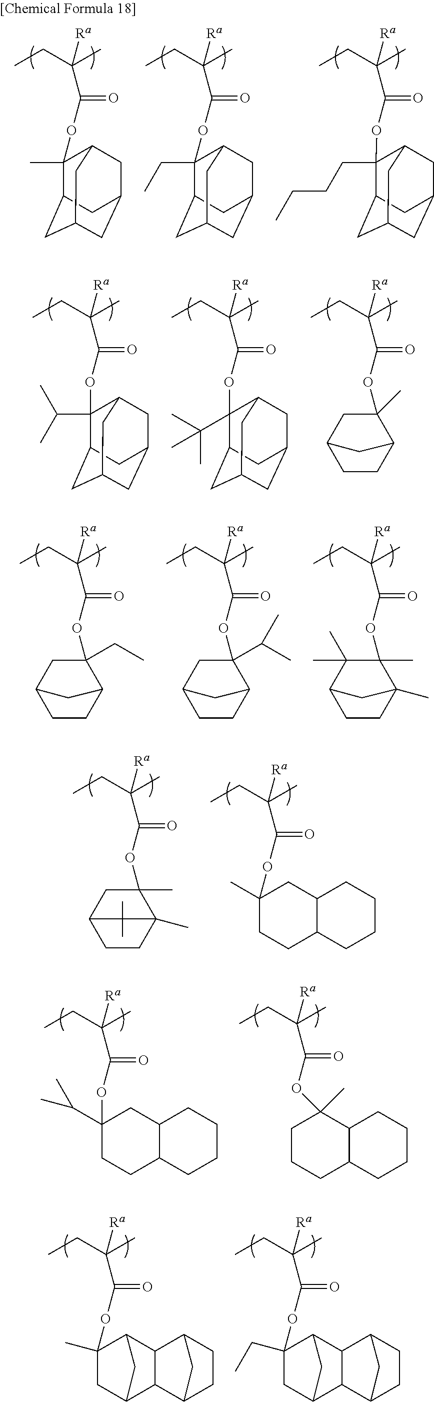

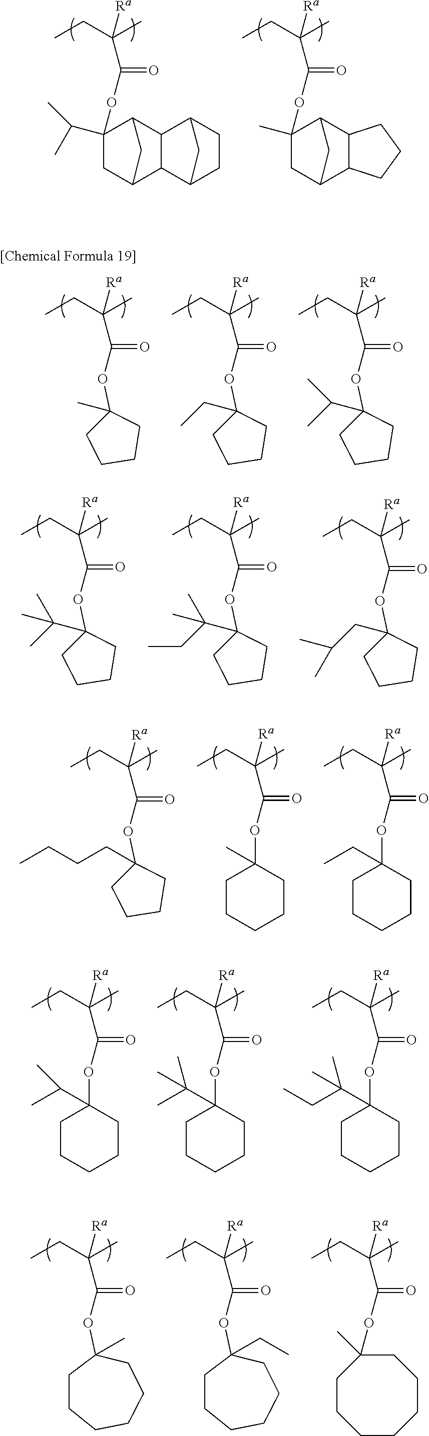

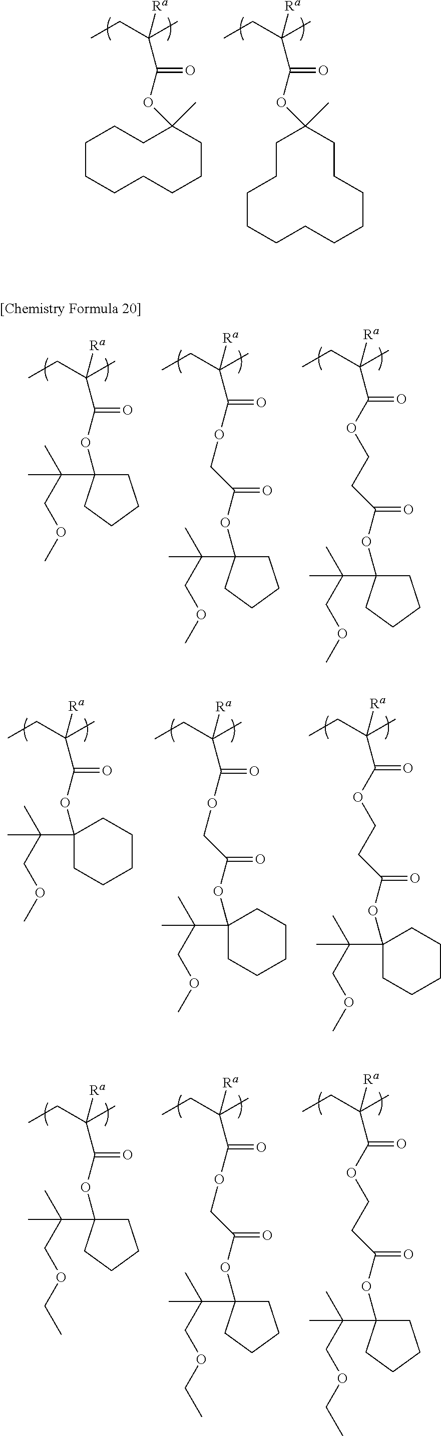

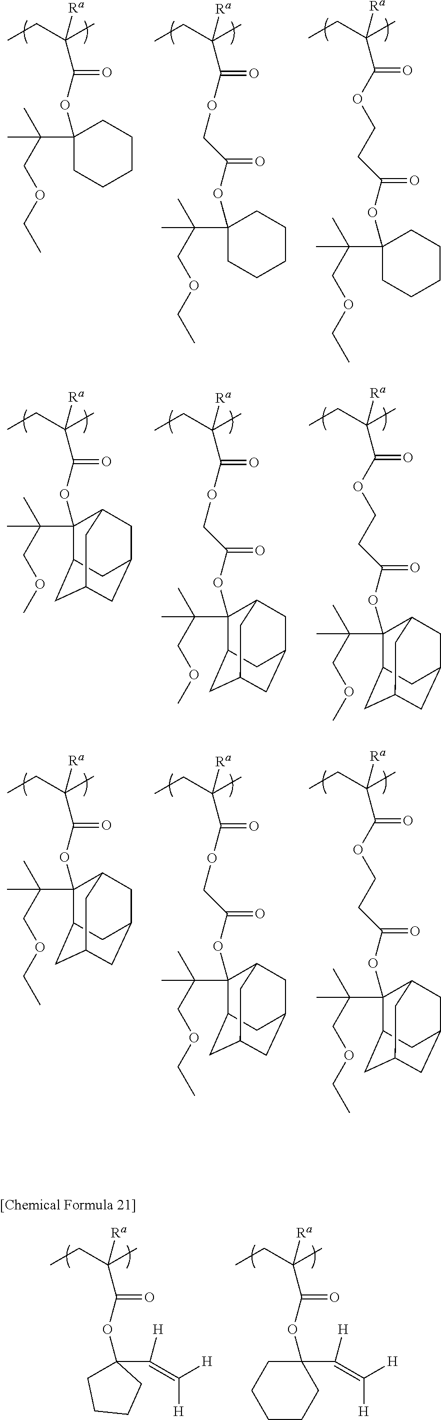

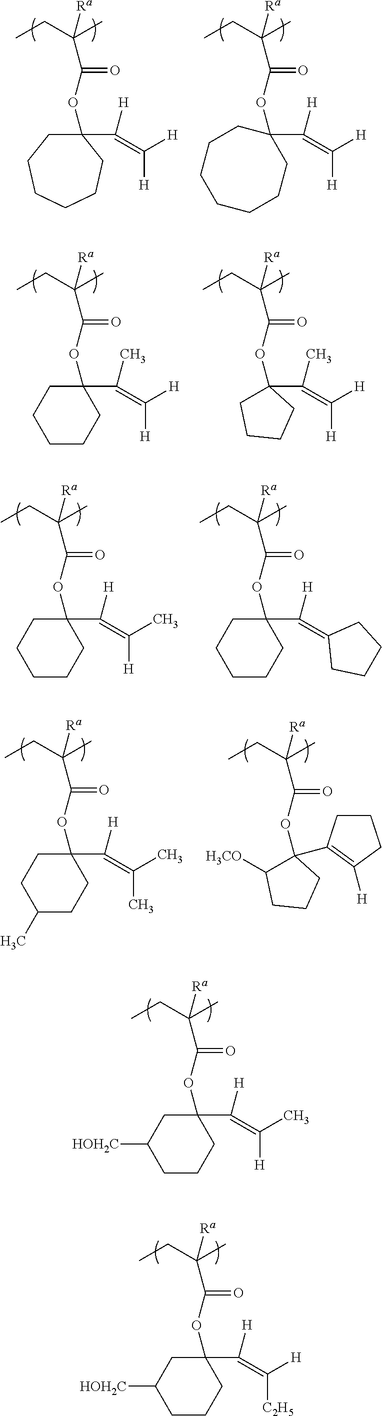

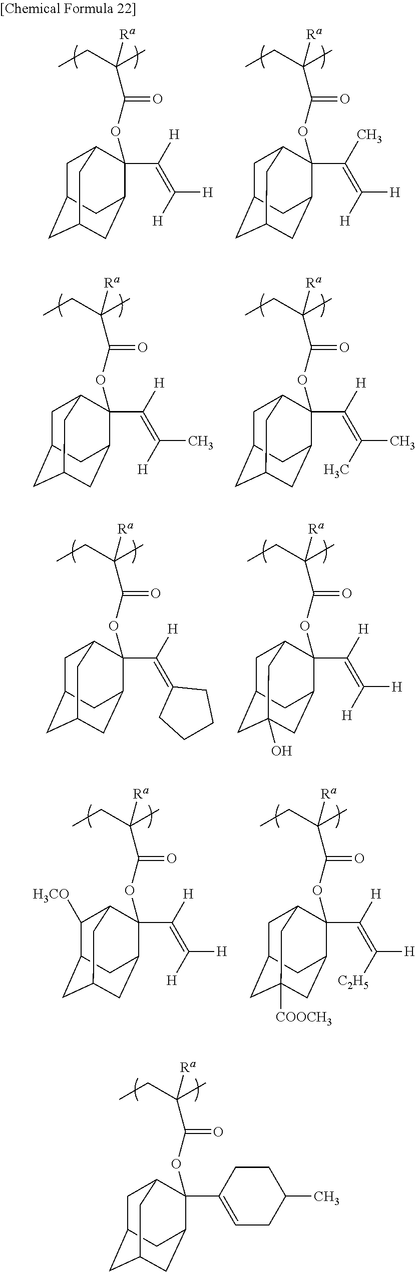

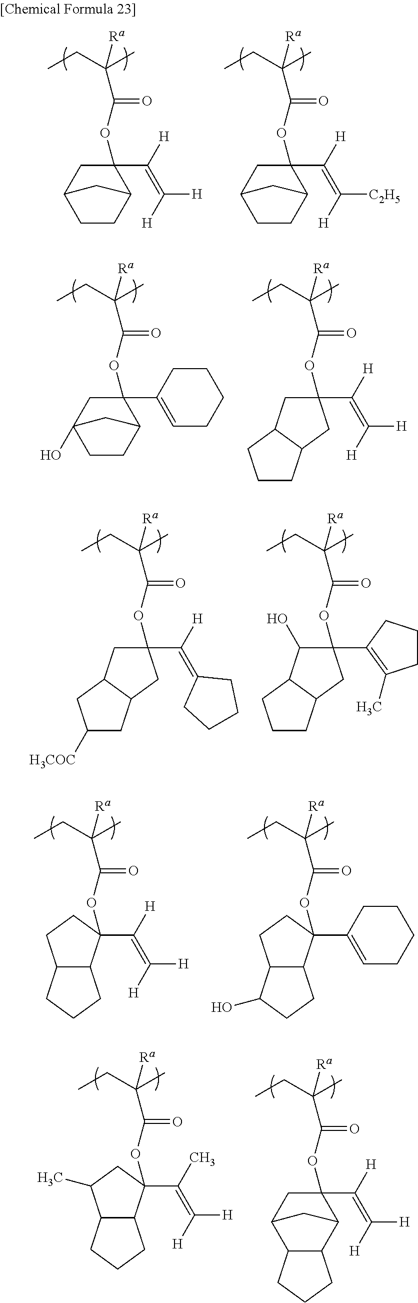

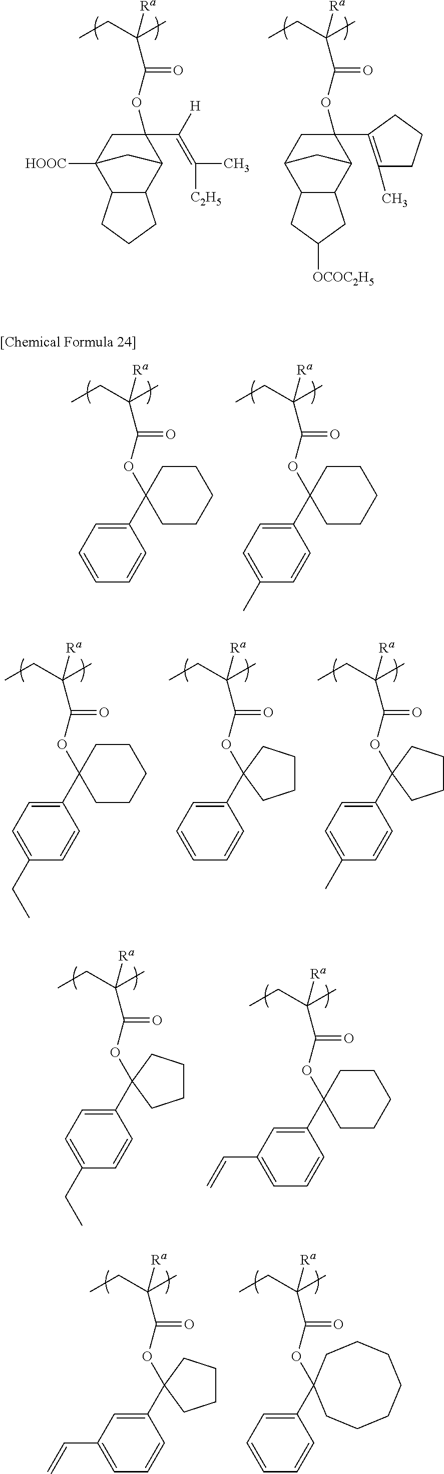

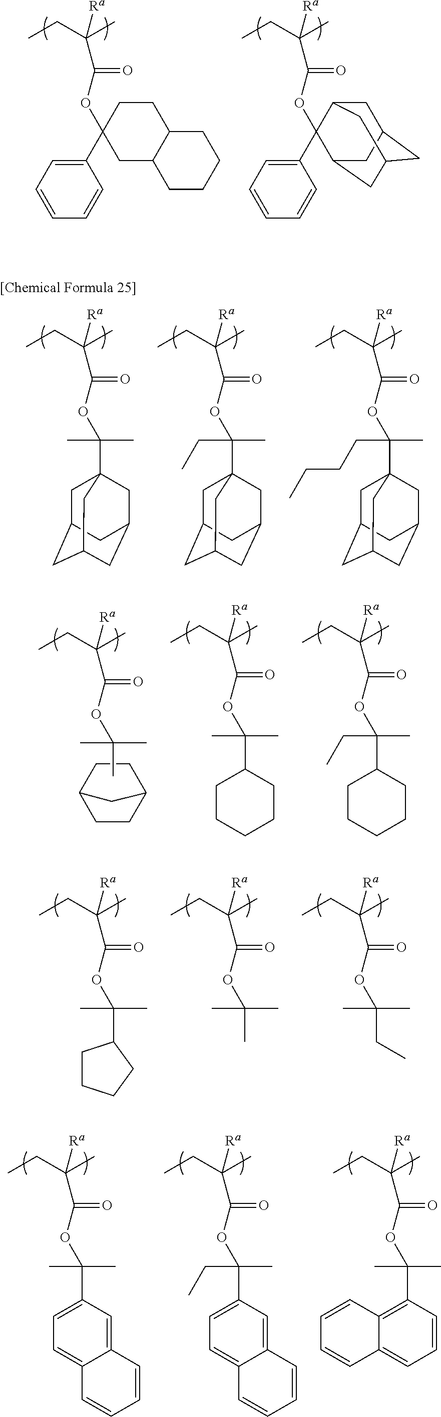

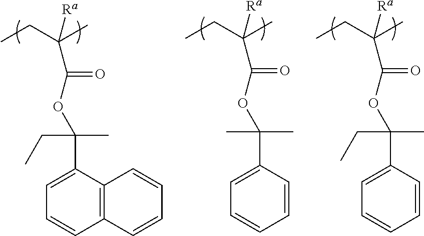

The constitutional unit (a1) included in the component (A1) may be used alone or two or more kinds thereof.

Since the lithography characteristics (LWR and the like) are easily improved using electron beams or EUV, a constitutional unit represented by Formula (a1-1) is more preferable as the constitutional unit (a1).

Among these, as the constitutional unit (a1), those having a constitutional unit represented by General Formula (a1-1-1) are particularly preferable.

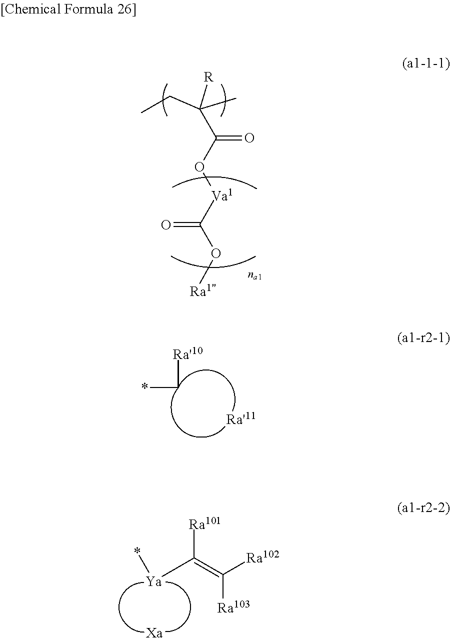

[In the formulae, Ra¹″ represents an acid dissociable group represented by General Formula (a1-r2-1) or (a1-r2-2). * represents a bonding site.]

In Formula (a1-1-1), R, Va¹, and nal are each the same as R, Va¹, and nal in Formula (a1-1).

The description of the acid dissociable group represented by General Formula (a1-r2-1) or (a1-r2-2) is the same as described above. Among these, it is preferable to select those in which the acid dissociable group is a cyclic group, and more preferable to select an acid dissociable group represented by General Formula (a1-r2-1) since the reactivity is enhanced for EB or EUV, which is suitable.

The proportion of the constitutional unit (a1) in the component (A1) is preferably 10% to 90% by mole, more preferably 20% to 80% by mole, still more preferably 30% to 70% by mole, and particularly preferably 40% to 65% by mole with respect to the total amount (100% by mole) of all constitutional units constituting the component (A1).

In a case where the proportion of the constitutional unit (a1) is set to be equal to or greater than the lower limits of the above-described preferable ranges, lithography characteristics such as sensitivity, CDU, resolution, and reduction of roughness are improved. Further, in a case where the proportion of the constitutional unit (a1) is equal to or less than the upper limits of the above-described preferable ranges, the constitutional unit (a1) and other constitutional units can be balanced, and the various lithography characteristics are enhanced.

### <<Other Constitutional Units>>

The component (A1) may have other constitutional units as necessary in addition to the constitutional unit (a1) described above.

Examples of the other constitutional units include a constitutional unit (a10) represented by General Formula (a10-1) described below; a constitutional unit (a2) containing a lactone-containing cyclic group; a constitutional unit (a5) which generates an acid upon light exposure; a constitutional unit (a6) having acid diffusion controllability; and a constitutional unit (a8) derived from a compound represented by General Formula (a8-1) described below.

In regard to constitutional unit (a10):

The constitutional unit (a10) is a constitutional unit represented by General Formula (a10-1) (here, a constitutional unit corresponding to the constitutional unit (a1) is excluded).

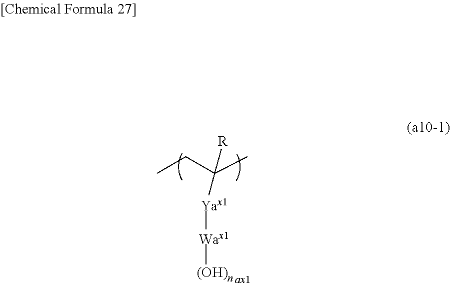

[In the formula, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent. nₐₓ₁ represents an integer of 1 or greater.]

In Formula (a10-1), R represents a hydrogen atom, an alkyl group having 1 to 5 carbon atoms, or a halogenated alkyl group having 1 to 5 carbon atoms.

R represents preferably a hydrogen atom, an alkyl group having 1 to 5 carbon atoms, or a fluorinated alkyl group having 1 to 5 carbon atoms, and from the viewpoint of industrial availability, R represents more preferably a hydrogen atom, a methyl group, or trifluoromethyl group, still more preferably a hydrogen atom or a methyl group, and particularly preferably a hydrogen atom.

In Formula (a10-1), Ya^{x1} represents a single bond or a divalent linking group.

In the chemical formula, the divalent linking group as Ya^{x1} is not particularly limited, and suitable examples thereof include a divalent hydrocarbon group which may have a substituent and a divalent linking group having a heteroatom.

Ya^{x1} represents preferably a single bond, an ester bond [—C(═O)—O— or —O—C(═O)—], an ether bond (—O—), a linear or branched alkylene group, or a combination thereof and more preferably a single bond or an ester bond [—C(═O)—O— or —O—C(═O)—].

In Formula (a10-1), Wa^{x1} represents an aromatic hydrocarbon group which may have a substituent.

Examples of the aromatic hydrocarbon group as Wa^{x1} include a group in which (nₐₓ₁+1) hydrogen atoms have been removed from an aromatic ring which may have a substituent. Here, the aromatic ring is not particularly limited as long as the aromatic ring is a cyclic conjugated system having (4n+2) π electrons. The aromatic ring has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the aromatic hydrocarbon ring with a heteroatom. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Examples of the aromatic hydrocarbon group as Wa^{x1} also include a group in which (nₐₓ₁+1) hydrogen atoms have been removed from an aromatic compound including an aromatic ring (for example, biphenyl and fluorene) which may have two or more substituents.

Among these, Wa^{x1} represents preferably a group in which (nₐₓ₁+1) hydrogen atoms have been removed from benzene, naphthalene, anthracene, or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group as Wa^{x1} may or may not have a substituent. Examples of the substituent include an alkyl group, an alkoxy group, a halogen atom, and a halogenated alkyl group. Examples of the alkyl group, the alkoxy group, the halogen atom, and the halogenated alkyl group as the substituent include the same groups as those for the substituent of the cyclic alicyclic hydrocarbon group as Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. It is preferable that the aromatic hydrocarbon group as Wa^{x1} has no substituent.

In Formula (a10-1), nₐₓ₁ represents an integer of 1 or greater, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are shown below.

In the formulae shown below, R^{α} represents a hydrogen atom, a methyl group, or a trifluoromethyl group.

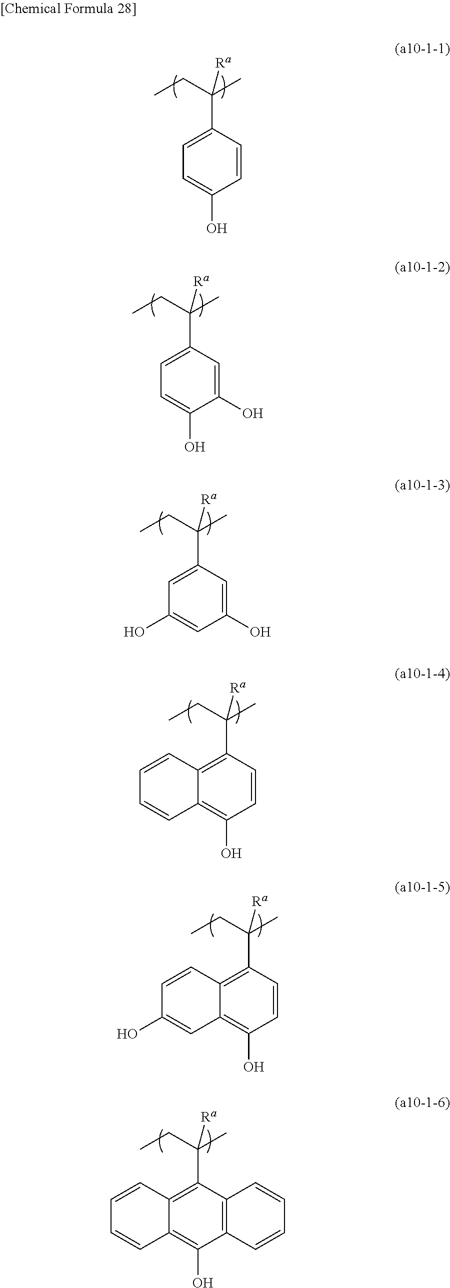

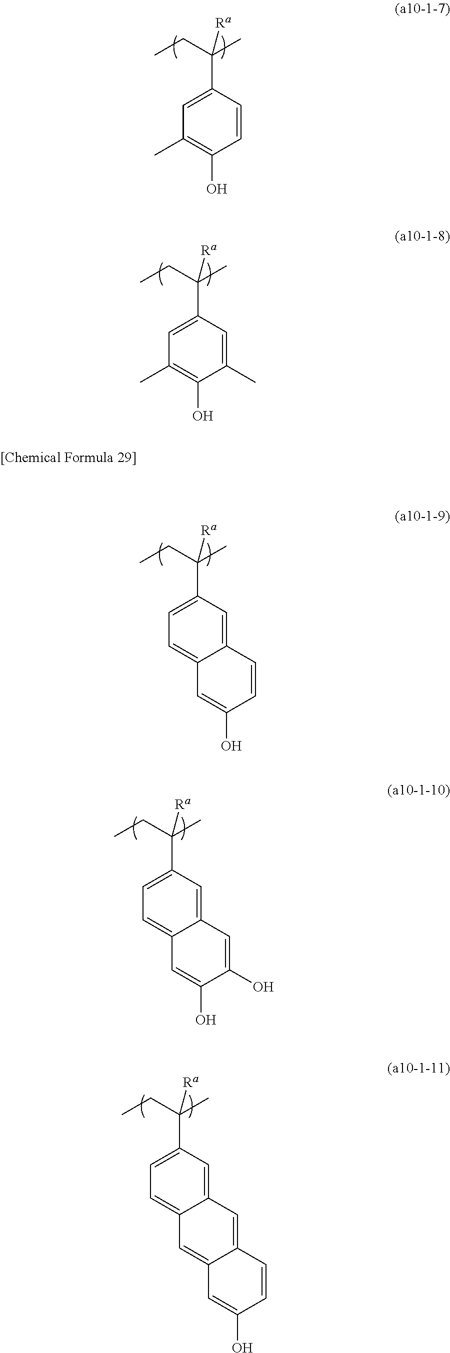

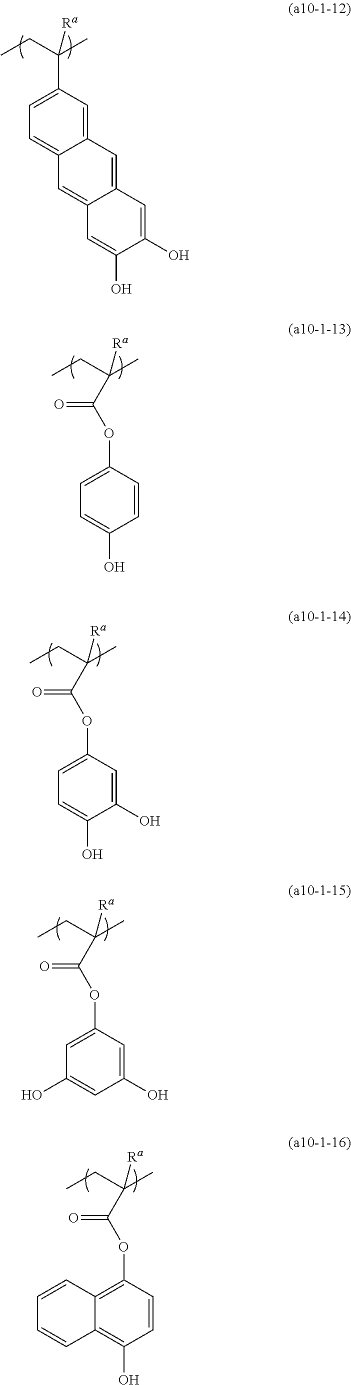

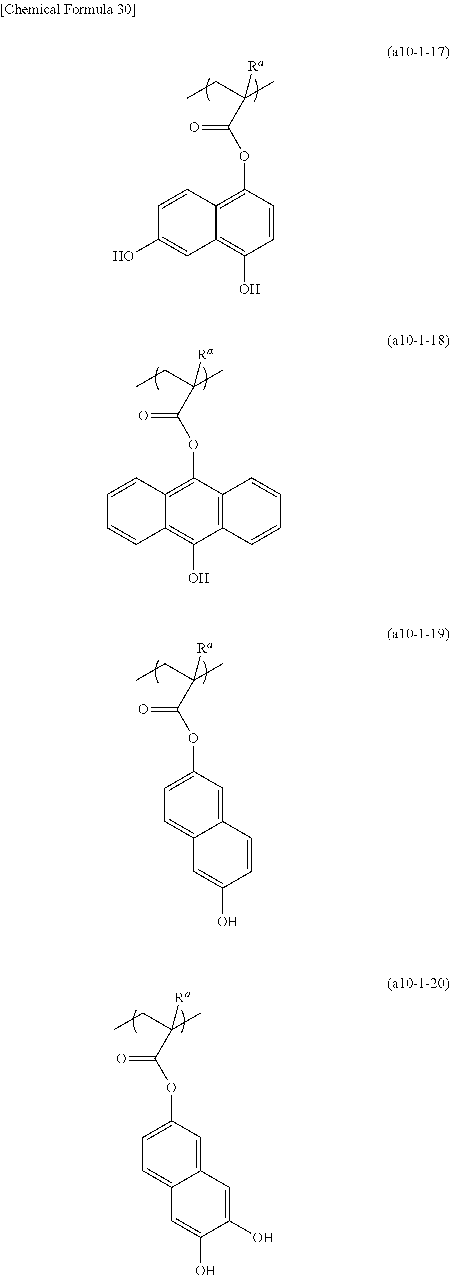

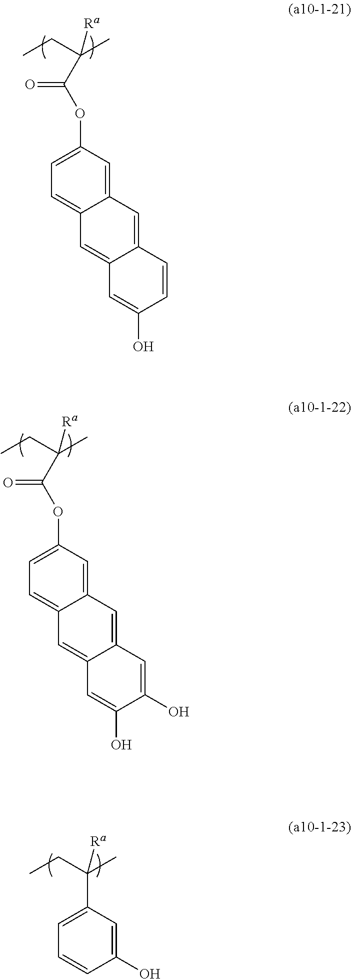

The constitutional unit (a10) that can be included in the component (A1) may be used alone or two or more kinds thereof.

In a case where the component (A1) has the constitutional unit (a10), the proportion of the constitutional unit (a10) in the component (A1) is preferably 20% to 80% by mole, more preferably 30% to 70% by mole, and still more preferably 35% to 60% by mole with respect to the total amount (100% by mole) of all constitutional units constituting the component (A1).

In a case where the proportion of the constitutional unit (a10) is set to be equal to or greater than the lower limits, the sensitivity is likely to be enhanced. Meanwhile, in a case where the proportion of the constitutional unit (a10) is set to be equal to or less than the upper limits, the constitutional unit (a10) and other constitutional units are likely to be balanced.

In regard to constitutional unit (a2):

The component (A1) may further have a constitutional unit (a2) (here, a constitutional unit corresponding to the constitutional unit (a1) is excluded) containing a lactone-containing cyclic group.

In a case where the component (A1) is used to form a resist film, the lactone-containing cyclic group of the constitutional unit (a2) is effective for increasing the adhesiveness of the resist film to the substrate. Further, in a case where the component (A1) contains the constitutional unit (a2), for example, the lithography characteristics and the like are enhanced due to the effects of appropriately adjusting the acid diffusion length, increasing the adhesiveness of the resist film to the substrate, and appropriately adjusting the solubility during the development.

The term “lactone-containing cyclic group” indicates a cyclic group that has a ring (lactone ring) containing —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and an optional lactone-containing cyclic group can be used. Specific examples thereof include groups each represented by General Formulae (a2-r-1) to (a2-r-7).

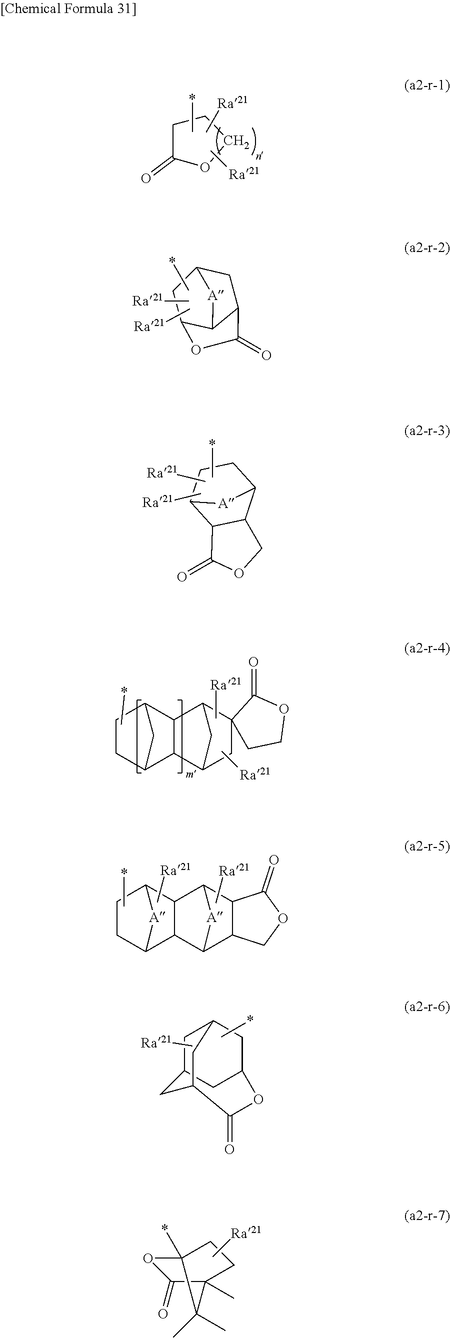

[In the formulae, Ra′²¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, or a lactone-containing cyclic group, A″ represents an alkylene group having 1 to 5 carbon atoms which may have an oxygen atom (—O—) or a sulfur atom (—S—), an oxygen atom, or a sulfur atom, n′ represents an integer of 0 to 2, and m′ represents 0 or 1. * represents a bonding site (the same applies hereinafter).]

In General Formulae (a2-r-1) to (a2-r-7), as the alkyl group as Ra′²¹, an alkyl group having 1 to 6 carbon atoms is preferable. Further, it is preferable that the alkyl group is linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

As the alkoxy group as Ra′²¹, an alkoxy group having 1 to 6 carbon atoms is preferable. Further, it is preferable that the alkoxy group is linear or branched. Specific examples thereof include a group formed by linking the alkyl group described as the alkyl group as Ra′²¹ to an oxygen atom (—O—).

As the halogen atom as Ra′²¹, a fluorine atom is preferable.

Examples of the halogenated alkyl group as Ra′²¹ include groups in which some or all hydrogen atoms in the alkyl group as Ra′²¹ have been substituted with the halogen atoms. As the halogenated alkyl group, a fluorinated alkyl group is preferable, and a perfluoroalkyl group is particularly preferable.

In —COOR″ and —OC(═O)R″ as Ra′²¹, each R″ represents a hydrogen atom, an alkyl group, or a lactone-containing cyclic group.

The alkyl group as R″ may be linear, branched, or cyclic and has preferably 1 to 15 carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, an alkyl group having 3 to 15 carbon atoms is preferable, an alkyl group having 4 to 12 carbon atoms is more preferable, and an alkyl group having 5 to 10 carbon atoms is most preferable. Specific examples thereof include a group in which one or more hydrogen atoms have been removed from a monocycloalkane, which may or may not be substituted with a fluorine atom or a fluorinated alkyl group; and a group in which one or more hydrogen atoms have been removed from a polycycloalkane such as bicycloalkane, tricycloalkane, or tetracycloalkane. More specific examples thereof include a group in which one or more hydrogen atoms have been removed from a monocycloalkane such as cyclopentane or cyclohexane; and a group in which one or more hydrogen atoms have been removed from a polycycloalkane such as adamantane, norbornane, isobornane, tricyclo[5.2.1.0^{2,6}]decane, or tetracyclododecane.

Examples of the lactone-containing cyclic group as R″ include the same groups as those for the groups each represented by General Formulae (a2-r-1) to (a2-r-7).

As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among these, it is preferable that Ra′²¹'s each independently represent a hydrogen atom or a cyano group.

In General Formulae (a2-r-2), (a2-r-3), and (a2-r-5), as the alkylene group having 1 to 5 carbon atoms as A″, a linear or branched alkylene group is preferable, and examples thereof include a methylene group, an ethylene group, an n-propylene group, and an isopropylene group. Specific examples of the alkylene groups that contain an oxygen atom or a sulfur atom include groups in which —O— or —S— is interposed in the terminal of the alkylene group or between the carbon atoms of the alkylene group, and examples thereof include —O—CH₂—, —CH₂—O—CH₂—, —S—CH₂—, and —CH₂—S—CH₂—. As A″, an alkylene group having 1 to 5 carbon atoms or —O— is preferable, an alkylene group having 1 to 5 carbon atoms is more preferable, and a methylene group is most preferable.

Specific examples of the groups each represented by General Formulae (a2-r-1) to (a2-r-7) are shown below.

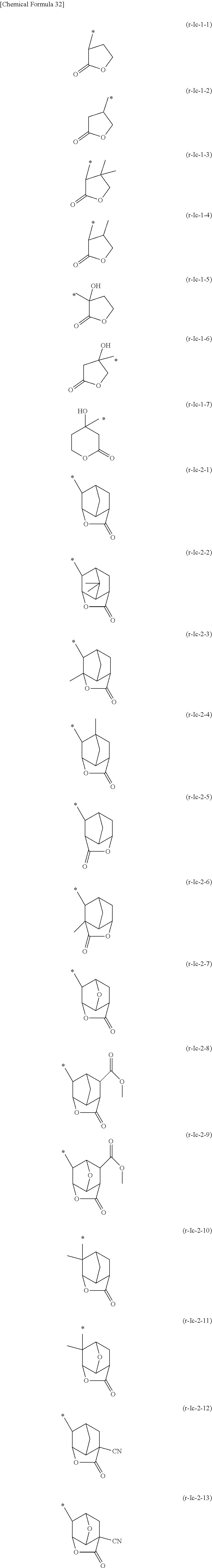

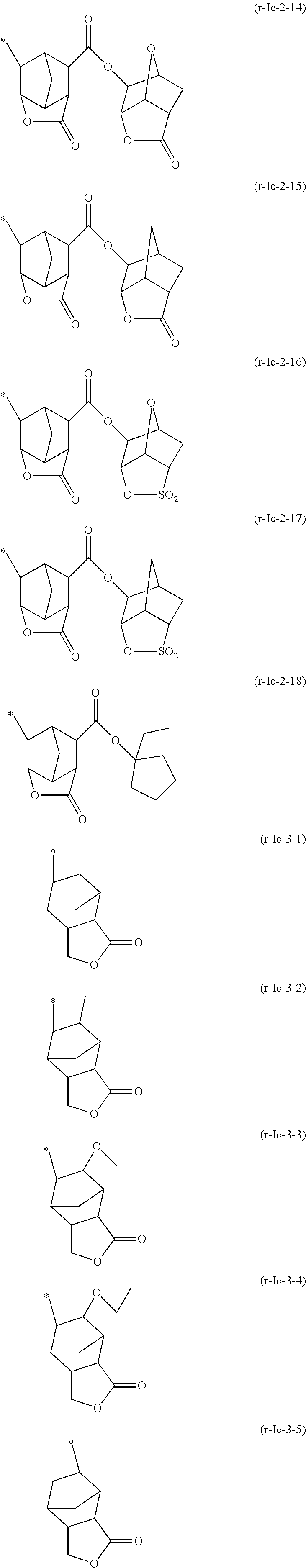

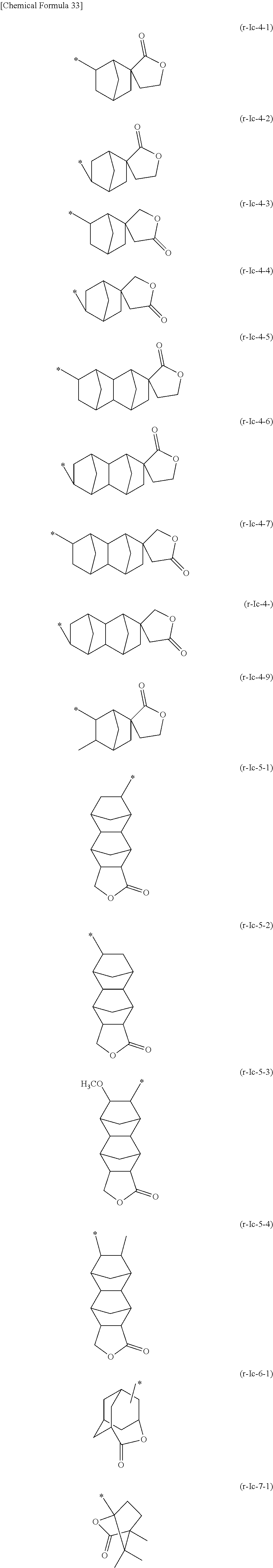

As the constitutional unit (a2), a constitutional unit derived from acrylic acid ester in which the hydrogen atom bonded to the carbon atom at the α-position may be substituted with a substituent is preferable.

It is preferable that such a constitutional unit (a2) is a constitutional unit represented by General Formula (a2-1).

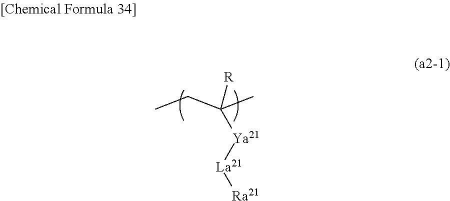

[In the formula, R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO—, or —CONHCS—, and R′ represents a hydrogen atom or a methyl group. However, in a case where La²¹ represents —O—, Ya²¹ does not represent —CO—. Ra²¹ represents a lactone-containing cyclic group.]

In Formula (a2-1), R has the same definition as described above. R represents preferably a hydrogen atom, an alkyl group having 1 to 5 carbon atoms, or a fluorinated alkyl group having 1 to 5 carbon atoms, and from the viewpoint of industrial availability, particularly preferably a hydrogen atom or a methyl group.

In Formula (a2-1), the divalent linking group as Ya²¹ is not particularly limited, and suitable examples thereof include a divalent hydrocarbon group which may have a substituent, and a divalent linking group having a heteroatom.

As Ya²¹, a single bond, an ester bond [—C(═O)—O—], an ether bond (—O—), a linear or branched alkylene group, or a combination thereof is preferable.

In Formula (a2-1), it is preferable that Ya²¹ represents a single bond and La²¹ represents —COO— or —OCO—.

In Formula (a2-1), Ra²¹ represents a lactone-containing cyclic group.

Suitable examples of the lactone-containing cyclic group as Ra²¹ include groups each represented by General Formulae (a2-r-1) to (a2-r-7).

The constitutional unit (a2) that can be included in the component (A1) may be used alone or two or more kinds thereof.

In a case where the component (A1) has the constitutional unit (a2), the proportion of the constitutional unit (a2) is preferably 1% to 20% by mole, more preferably 1% to 15% by mole, and still more preferably 1% to 10% by mole with respect to the total amount (100% by mole) of all constitutional units constituting the component (A1).

In a case where the proportion of the constitutional unit (a2) is set to be equal to or greater than the preferable lower limits, the effect to be obtained by allowing the component (A1) to have the constitutional unit (a2) is sufficiently obtained by the above-described effects. Further, in a case where the proportion thereof is set to be equal to or less than the upper limits, the constitutional unit (a2) and other constitutional units can be balanced, and the various lithography characteristics are enhanced.

Constitutional unit (a5):

The component (A1) may or may not have the constitutional unit (a5) which generates an acid upon light exposure. As the constitutional unit (a5), a known constitutional unit can be used. In a case where the component (A1) has the constitutional unit (a5), the acid generated upon light exposure is likely to be uniformly distributed in the resist film. Examples of the constitutional unit (a5) include a constitutional unit including a structure described in the component (B) described below. Examples thereof include a constitutional unit including a structure represented by any of General Formulae (b-1) to (b-3) described below.

Suitable examples of the constitutional unit (a5) include a constitutional unit represented by General Formula (a5-1).

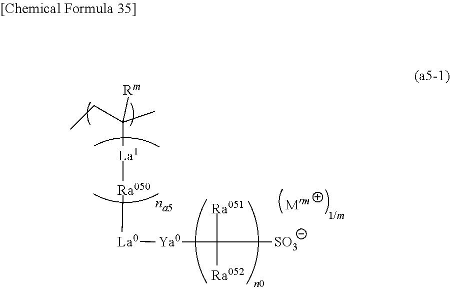

[In the formula, R™ represents an alkyl group having 1 to 5 carbon atoms, a halogenated alkyl group having 1 to 5 carbon atoms, a halogen atom, or a hydrogen atom. La⁵ represents a divalent linking group or a single bond. Ra⁵⁰ represents a divalent hydrocarbon group which may have a substituent. nₐ₅ represents an integer of 0 to 2. La⁵ represents a divalent linking group. Ya⁵ represents a divalent linking group which may have a heteroatom or a single bond. Ra⁵¹ and Ra⁵² each independently represent a hydrogen atom, a fluorine atom, or a fluorinated alkyl group. n5 represents an integer of 1 to 4. m represents an integer of 1 or greater, and M′^{m+} represents an m-valent onium cation.]

### {Anion Moiety}

In Formula (a5-1), R^{m} represents an alkyl group having 1 to 5 carbon atoms, a halogenated alkyl group having 1 to 5 carbon atoms, a halogen atom, or a hydrogen atom.

As the alkyl group having 1 to 5 carbon atoms as R^{m}, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group.

The halogenated alkyl group having 1 to 5 carbon atoms is a group in which some or all hydrogen atoms in the alkyl group having 1 to 5 carbon atoms have been substituted with halogen atoms.

Examples of the halogen atom include a fluorine atom, a chlorine atom, a bromine atom, and an iodine atom. As the halogen atom in the halogenated alkyl group, a fluorine atom is particularly preferable.

R™ represents preferably a hydrogen atom, an alkyl group having 1 to 5 carbon atoms, or a fluorinated alkyl group having 1 to 5 carbon atoms, and from the viewpoint of industrial availability, most preferably a hydrogen atom or a methyl group.

In Formula (a5-1), La⁵ represents a divalent linking group or a single bond.

The divalent linking group as La⁵ is not particularly limited, and suitable examples thereof include a divalent hydrocarbon group which may have a substituent and a divalent linking group having a heteroatom, each of which is the same as the divalent hydrocarbon group which may have a substituent and the divalent linking group having a heteroatom, described as the divalent linking group as Ya^{x1}.

Among these, La⁵ represents preferably an ester bond [—C(═O)—O— or —O—C(═O)—], an ether bond (—O—), a linear or branched alkylene group, an aromatic hydrocarbon group, or a combination thereof, or a single bond. Among these, Las represents more preferably an ester bond [—C(═O)—O— or —O—C(═O)—] or a single bond and still more preferably an ester bond [—C(═O)—O— or —O—C(═O)—].

In Formula (a5-1), Ra⁵⁰ represents a divalent hydrocarbon group which may have a substituent.

The divalent hydrocarbon group as Ra⁵⁰ may be an aliphatic hydrocarbon group or an aromatic hydrocarbon group.
· Aliphatic hydrocarbon group as Ra⁵⁰

The aliphatic hydrocarbon group means a hydrocarbon group that has no aromaticity. The aliphatic hydrocarbon group may be saturated or unsaturated. In general, it is preferable that the aliphatic hydrocarbon group is saturated.

Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable. Specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

The linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a heteroatom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. Examples of the linear or branched aliphatic hydrocarbon group include the same groups as those described above.

The cyclic aliphatic hydrocarbon group has preferably 3 to 20 carbon atoms and more preferably 3 to 12 carbon atoms.

The cyclic aliphatic hydrocarbon group may be a polycyclic group or a monocyclic group. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable, and specific examples thereof include adamantane, norbornane, isobornane, tricyclo[5.2.1.0^{2,6}]decane, and tetracyclododecane.

The cyclic aliphatic hydrocarbon group may or may not have a substituent.

Examples of the substituent include an alkyl group, an alkoxy group, a halogen atom, a halogenated alkyl group, a hydroxyl group, and a carbonyl group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

Examples of the halogen atom as the substituent include a fluorine atom, a chlorine atom, a bromine atom, and an iodine atom, and a fluorine atom is preferable.

Examples of the halogenated alkyl group as the substituent include groups in which some or all hydrogen atoms in the alkyl groups have been substituted with the halogen atoms.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

Aromatic hydrocarbon group as Ra⁵⁰

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms. Specific examples of the aromatic ring include an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group in which two hydrogen atoms have been removed from the aromatic hydrocarbon ring or aromatic heterocyclic ring (an arylene group or a heteroarylene group); a group in which two hydrogen atoms have been removed from an aromatic compound having two or more aromatic rings (for example, biphenyl or fluorene); and a group in which one hydrogen atom of a group (an aryl group or a heteroaryl group) obtained by removing one hydrogen atom from the aromatic hydrocarbon ring or aromatic heterocyclic ring has been substituted with an alkylene group (for example, a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The alkylene group bonded to the aryl group or the heteroaryl group has preferably 1 to 4 carbon atoms, more preferably 1 or 2 carbon atoms, and particularly preferably 1 carbon atom.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of the substituent include an alkyl group, an alkoxy group, a halogen atom, a halogenated alkyl group, and a hydroxyl group.

As the alkyl group as the substituent, an alkyl group having 1 to 5 carbon atoms is preferable, and a methyl group, an ethyl group, a propyl group, an n-butyl group, or a tert-butyl group is most preferable.

Examples of the alkoxy group, the halogen atom, and the halogenated alkyl group as the substituents include those described as the substituents that substitute a hydrogen atom contained in the cyclic aliphatic hydrocarbon group.

In Formula (a5-1), nₐ₅ represents an integer of 0 to 2.

Among these, Ra⁵⁰ represents preferably an aliphatic hydrocarbon group having a ring in the structure thereof, more preferably a cyclic aliphatic hydrocarbon group which may have a substituent having a heteroatom in the ring structure thereof, and still more preferably an alicyclic hydrocarbon group which may have a substituent, which is a polycyclic group or a monocyclic group.

Alternatively, among these, Ra⁵⁰ is preferably an aromatic hydrocarbon group.

In a case where nₐ₅ is 2, both of two Ra⁵⁰'s may be an alicyclic hydrocarbon group which may have a substituent, may be an aromatic hydrocarbon group, or may be a combination of an alicyclic hydrocarbon group which may have a substituent and an aromatic hydrocarbon group.

In Formula (a5-1), La⁵ represents a divalent linking group.

Examples of the divalent linking group as La⁵ include a non-hydrocarbon oxygen atom-containing linking group such as an oxygen atom (ether bond: —O—), an ester bond (—C(═O)—O—), an oxycarbonyl group (—O—C(—O)—), an amide bond (—C(═O)—NH—), a carbonyl group (—C(═O)—), or a carbonate bond (—O—C(═O)—O—); and combinations of the non-hydrocarbon oxygen atom-containing linking groups with an alkylene group. Further, a sulfonyl group (—SO₂—) may be linked to the combination.

Examples of such a divalent linking group include linking groups each represented by General Formulae (L-a1-1) to (L-a1-8). Further, in General Formulae (L-a1-1) to (L-a1-8), V′¹⁰¹ in General Formulae (L-a1-1) to (L-a1-8) is bonded to Ra⁵⁰ in Formula (a5-1).

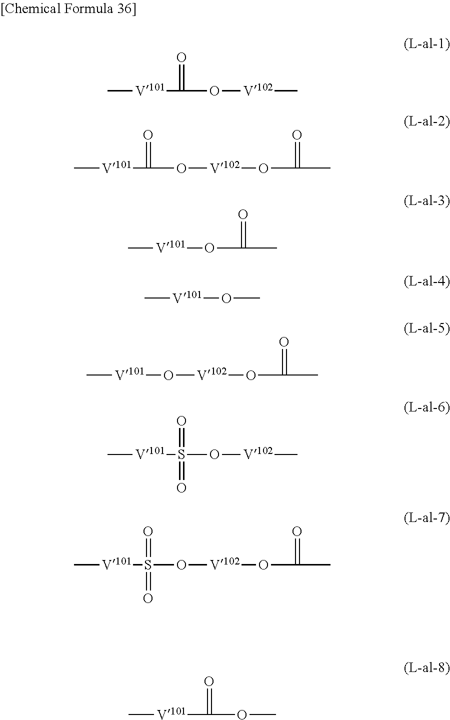

[In the formulae, V′¹⁰¹ represents a single bond or an alkylene group having 1 to 5 carbon atoms, and V′¹⁰² represents a divalent saturated hydrocarbon group having 1 to 30 carbon atoms.]

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

Further, a part of methylene group in the alkylene group as V′¹⁰¹ and V′¹⁰² may be substituted with a divalent aliphatic cyclic group having 5 to 10 carbon atoms. As the aliphatic cyclic group, a divalent group in which one hydrogen atom has been further removed from the cyclic aliphatic hydrocarbon group (a monocyclic aliphatic hydrocarbon group or a polycyclic aliphatic hydrocarbon group) as Ra′³ in Formula (a1-r-1) is preferable, and a cyclohexylene group, a 1,5-adamantylene group, or a 2,6-adamantylene group is more preferable.

La⁵ represents preferably a divalent linking group having an ester bond or a divalent linking group having an ether bond, more preferably linking groups each represented by Formulae (L-a1-1) to (L-a1-5) and (L-a1-8), and still more preferably a linking group represented by Formula (L-a1-3) or (L-a1-8).

In Formula (a5-1), Ya⁵ represents a divalent linking group which may have a heteroatom or a single bond.

The divalent linking group as Ya⁵ is not particularly limited, and suitable examples thereof include a divalent hydrocarbon group which may have a substituent, and a divalent linking group having a heteroatom.

The divalent hydrocarbon group which may have a substituent and the divalent linking group having a heteroatom as Ya⁵ are the same as the divalent hydrocarbon group which may have a substituent and the divalent linking group having a heteroatom, described as the divalent linking group as Ya^{x1}.

Among these, Ya⁵ represents preferably a linear or branched alkylene group or a single bond and more preferably a single bond.

In Formula (a5-1), Ra⁵¹ and Ra⁵² each independently represent a hydrogen atom, a fluorine atom, or a fluorinated alkyl group.

The fluorinated alkyl groups as Ra⁵¹ and Ra⁵² are each preferably a linear or branched fluorinated alkyl group having 1 to 5 carbon atoms and more preferably a trifluoromethyl group.

In Formula (a5-1), it is preferable that at least one of Ra⁵¹ and Ra⁵² bonded to the carbon atom adjacent to SO₃⁻ represents a fluorine atom from the viewpoint of acid strength.

In Formula (a5-1), n5 represents an integer of 1 to 4 and preferably 1, 2, or 3.

### {Cation Moiety}

In Formula (a5-1), M′^{m+} represents an m-valent onium cation. Among these, it is preferable that M′^{m+} represents a sulfonium cation or an iodonium cation. m represents an integer of 1 or greater.

Preferred examples of the cation moiety ((M′^{m1+})_{l/m}) include organic cations each represented by General Formulae (ca-1) to (ca-3).

As the cation moiety ((M′^{m+})_{l/m}) in Formula (a5-1), a sulfonium cation is preferable, the cations each represented by Formulae (ca-1) to (ca-3) are more preferable, the cation represented by Formula (ca-1) is still more preferable, and the cations each represented by Formulae (ca-1-1) to (ca-1-83) are particularly preferable.

In particular, from the viewpoint of improving the sensitivity, the suitable cation represented by Formula (ca-1) preferably has, as a substituent, an electron-withdrawing group such as a fluorine atom, a fluorinated alkyl group, or a sulfonyl group and is, for example, particularly preferably a cation selected from the group consisting of cations each represented by Chemical Formulae (ca-1-44) and (ca-1-71) to (ca-1-83).

Specific preferred examples of the constitutional unit (a5) are shown below.

In the formulae shown below, R^{α} represents a hydrogen atom, a methyl group, or a trifluoromethyl group. m and M′^{m+} are the same as m and M′^{m+} in General Formula (a5-1).

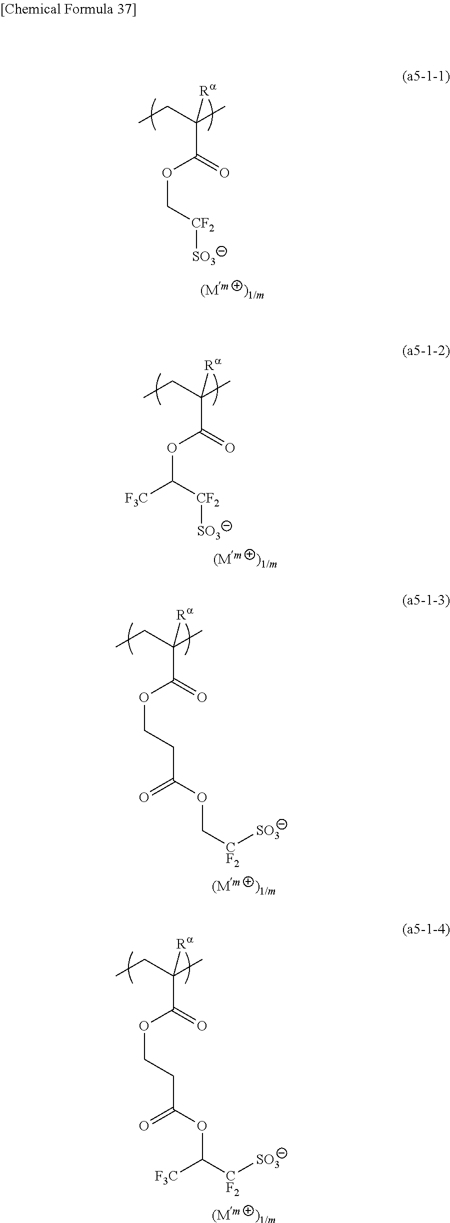

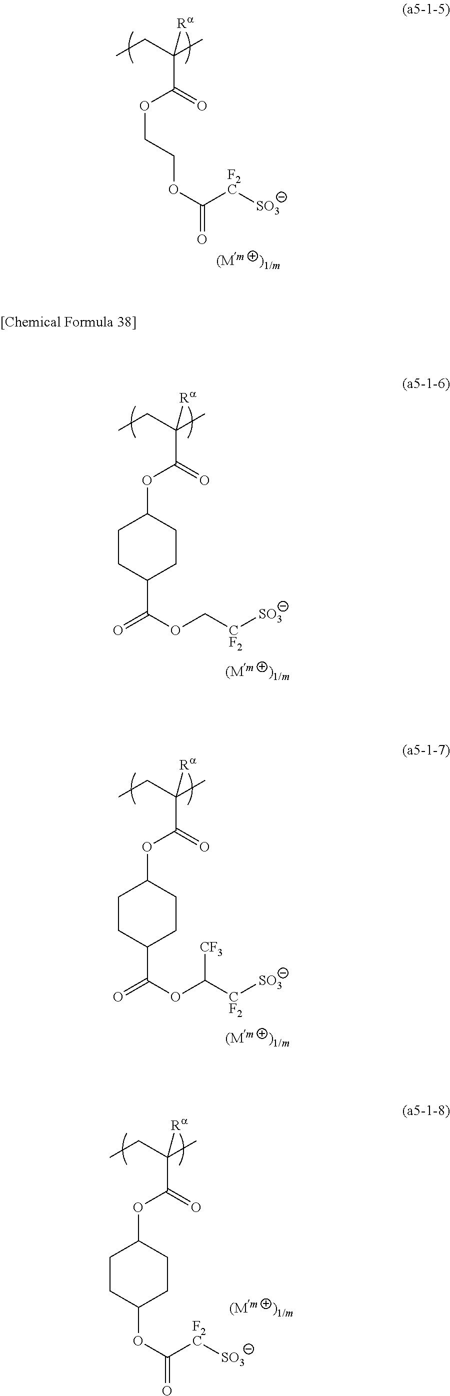

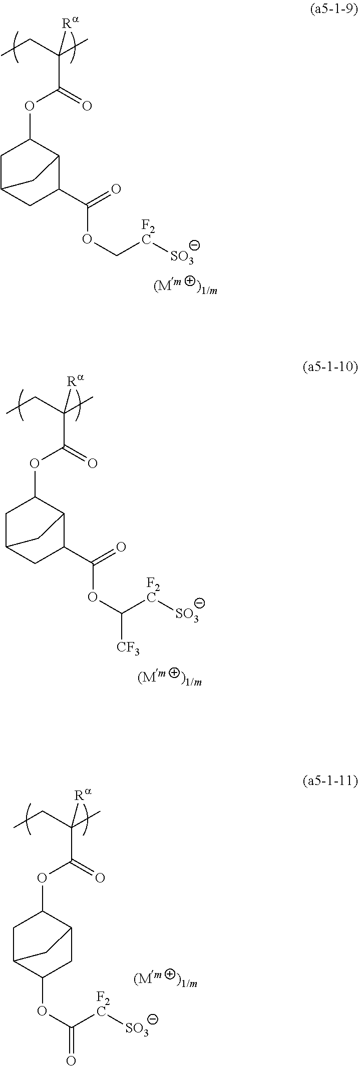

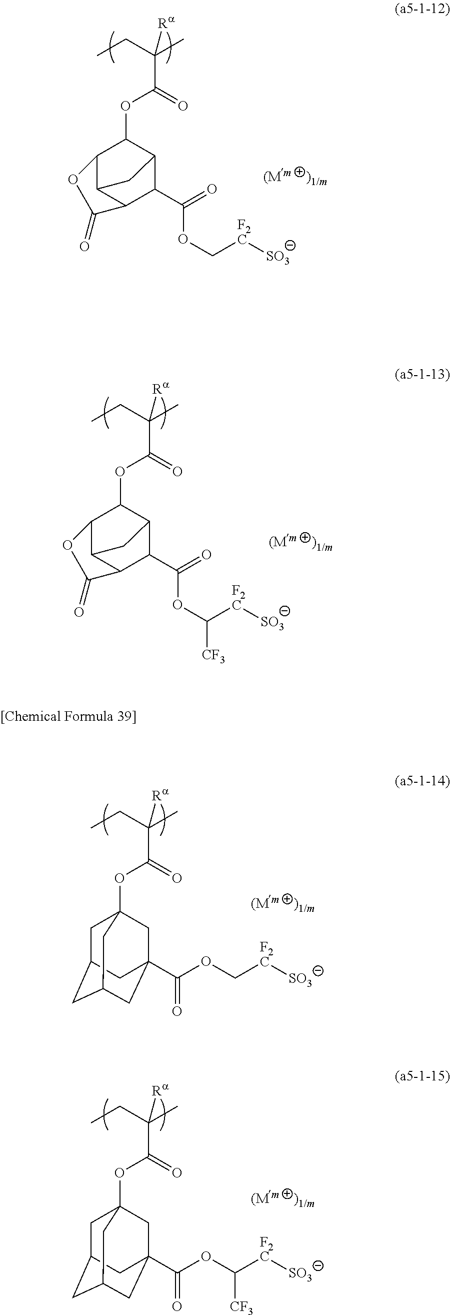

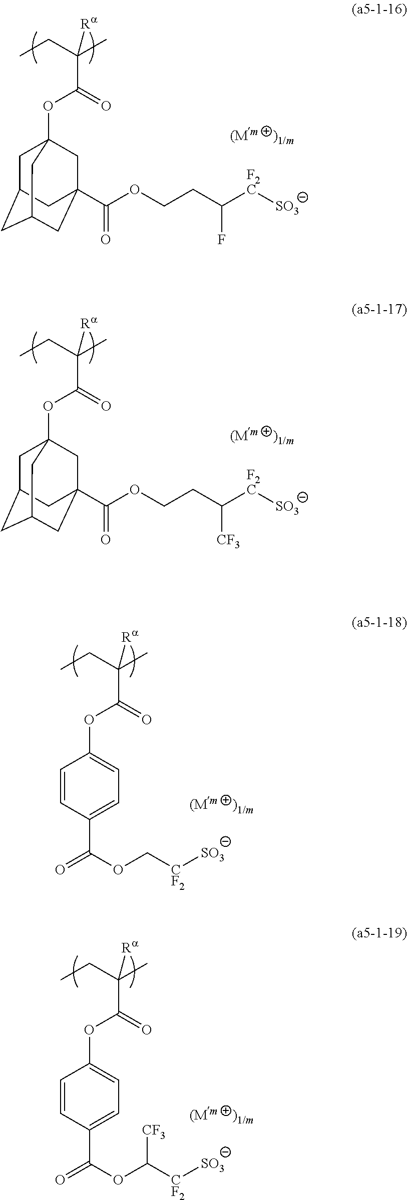

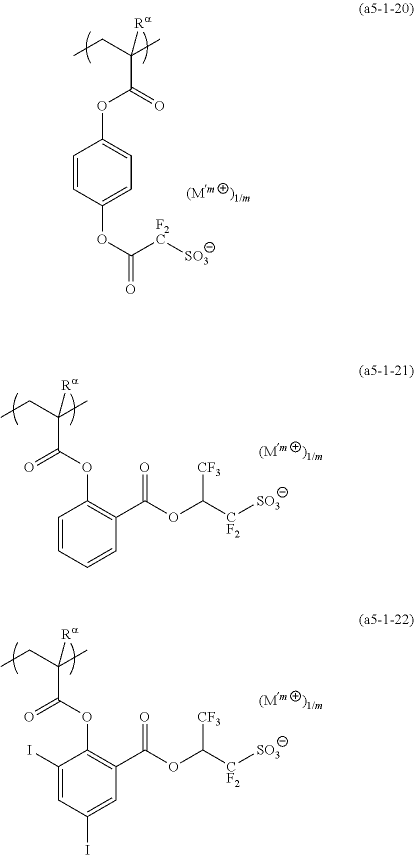

The constitutional unit (a5) included in the component (A1) may be used alone or two or more kinds thereof.

In a case where the component (A1) has the constitutional unit (a5), the proportion of the constitutional unit (a5) in the component (A1) is preferably 5% to 25% by mole, more preferably 10% to 20% by mole, and still more preferably 15% to 20% by mole with respect to the total amount (100% by mole) of all constitutional units constituting the component (A1).

In a case where the proportion of the constitutional unit (a5) is equal to or greater than the lower limits of the above-described preferable range, it is easy to realize further improvement of sensitivity and improvement of resolution. Meanwhile, in a case where the proportion of the constitutional unit (a5) is equal to or less than the upper limits of the above-described preferable ranges, the constitutional unit (a5) and other constitutional units are likely to be balanced.

Constitutional unit (a6):

The constitutional unit (a6) is a constitutional unit having acid diffusion controllability. The component (A1) may or may not have the constitutional unit (a6). As the constitutional unit (a6), a known constitutional unit can be used. Examples of the constitutional unit (a6) include a constitutional unit including a structure described in the component (D1) and the component (D2) described below. Examples thereof include a constitutional unit including a structure represented by any of General Formulae (d1-1) to (d1-3) described below.

The constitutional unit (a6) included in the component (A1) may be used alone or two or more kinds thereof.

In a case where the component (A1) has the constitutional unit (a6), the proportion of the constitutional unit (a6) in the component (A1) is preferably 1% to 20% by mole, more preferably 2% to 15% by mole, and still more preferably 3% to 10% by mole with respect to the total amount (100% by mole) of all constitutional units constituting the component (A1).

In a case where the proportion of the constitutional unit (a6) is equal to or greater than the lower limits of the above-described preferable range, it is easy to realize further improvement of sensitivity. Meanwhile, in a case where the proportion of the constitutional unit (a5) is equal to or less than the upper limits of the above-described preferable ranges, the constitutional unit (a5) and other constitutional units are likely to be balanced.

In regard to constitutional unit (a8):

The constitutional unit (a8) is a constitutional unit derived from a compound represented by General Formula (a8-1).

Here, constitutional units corresponding to the constitutional unit (a0) are excluded.

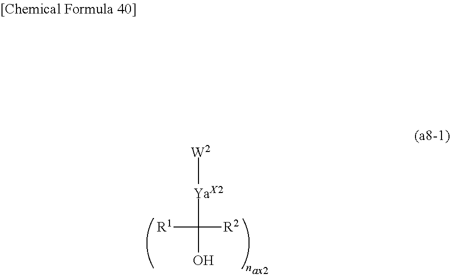

[In the formula, W² represents a polymerizable group-containing group. Ya^{x2} represents a single bond or an (nₐₓ₂+1)-valent linking group. Ya^{x2} and W² may form a condensed ring. R¹ represents a fluorinated alkyl group having 1 to 12 carbon atoms. R² represents an organic group having 1 to 12 carbon atoms, which may have a fluorine atom, or a hydrogen atom. R² and Ya^{x2} may be bonded to each other to form a ring structure. nₐₓ₂ represents an integer of 1 to 3.]

The term “polymerizable group” in the polymerizable group-containing group as W² is a group that enables a compound having the polymerizable group to be polymerized by radical polymerization or the like, which is, for example, a group having a multiple bond between carbon atoms, such as an ethylenic double bond.

The polymerizable group-containing group may be a group formed of only a polymerizable group or a group formed of a polymerizable group and a group other than the polymerizable group. Examples of the group other than the polymerizable group include a divalent hydrocarbon group which may have a substituent and a divalent linking group having a heteroatom.

Suitable examples of the polymerizable group-containing group include a group represented by a chemical formula: C(R^{X11})(R^{X12})═C(R^{X13})—Ya^{x0}-.

In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

Examples of the condensed ring formed by Ya^{x2} and W² include a condensed ring formed by a polymerizable group of the W² moiety and by Ya^{x2} and a condensed ring formed by a group other than the polymerizable group of the W² moiety and by Ya^{x2}.

The condensed ring formed by Ya^{x2} and W² may have a substituent.

Specific examples of the constitutional unit (a8) are shown below.

In the following formulae, R^{α} represents a hydrogen atom, a methyl group, or a trifluoromethyl group.

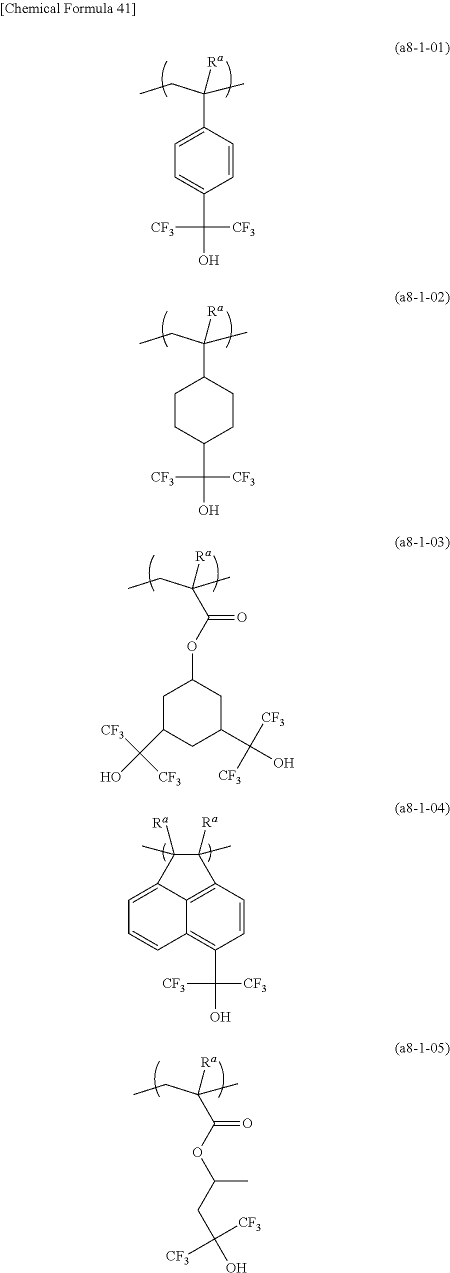

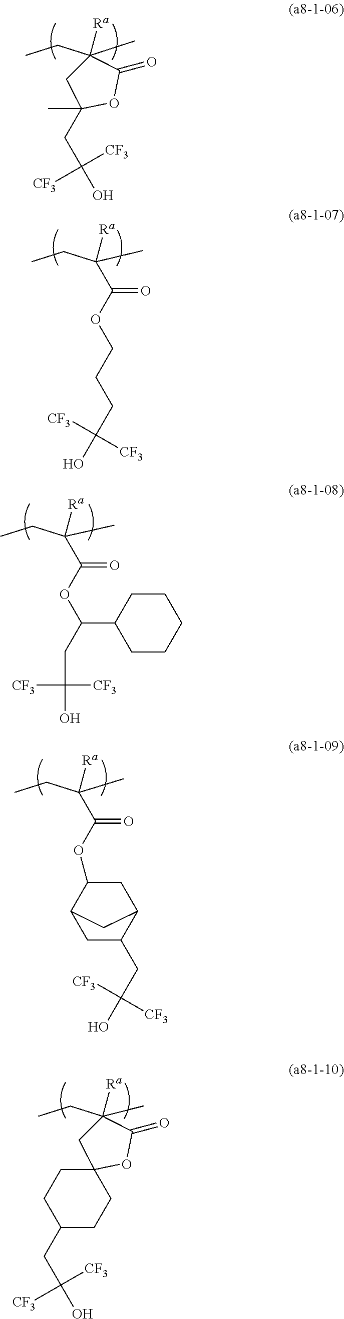

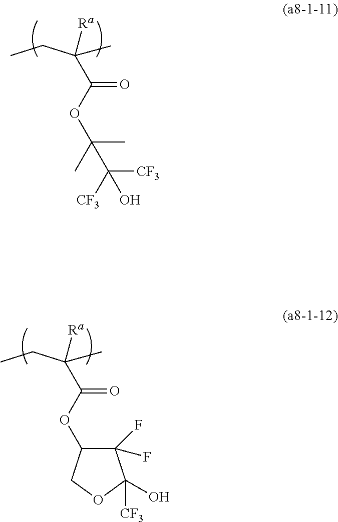

Among the examples, the constitutional unit (a8) is preferably at least one selected from the group consisting of constitutional units each represented by Chemical Formulae (a8-1-01) to (a8-1-04), (a8-1-06), (a8-1-08), (a8-1-09), and (a8-1-10), and more preferably at least one selected from the group consisting of constitutional units each represented by Chemical Formulae (a8-1-01) to (a8-1-04) and (a8-1-09).

The constitutional unit (a8) that can be included in the component (A1) may be used alone or two or more kinds thereof.

In a case where the component (A1) has the constitutional unit (a8), the proportion of the constitutional unit (a8) in the component (A1) is preferably 30% by mole or less, more preferably 0% to 20% by mole, and still more preferably 0% to 10% by mole with respect to the total amount (100% by mole) of all constitutional units constituting the component (A1).

The component (A1) contained in the resist composition may be used alone or in combination of two or more kinds thereof.

In the resist composition of the present embodiment, examples of the component (A1) include a polymer compound having a repeating structure of the constitutional unit (a1).

Among these, as the component (A1), a polymer compound having a repeating structure of the constitutional unit (a1) and the constitutional unit (a10) is preferable, and a polymer compound consisting of only a repeating structure of the constitutional unit (a1) and the constitutional unit (a10) is more preferable.

In the polymer compound having a repeating structure of the constitutional unit (a1) and the constitutional unit (a10), the proportion of the constitutional unit (a1) is preferably 10% to 90% by mole, more preferably 20% to 80% by mole, still more preferably 30% to 70% by mole, and particularly preferably 40% to 65% by mole with respect to the total amount (100% by mole) of all constitutional units constituting the polymer compound.

In addition, the proportion of the constitutional unit (a10) in the polymer compound is preferably 10% to 90% by mole, more preferably 20% to 80% by mole, still more preferably 30% to 70% by mole, and particularly preferably 35% to 60% by mole with respect to the total amount (100% by mole) of all constitutional units constituting the polymer compound.

Such a component (A1) can be produced by dissolving a monomer, from which each constitutional unit is derived, in a polymerization solvent and adding thereto a radical polymerization initiator such as azobisisobutyronitrile (AIBN) or dimethyl azobisisobutyrate (for example, V-601) to carry out the polymerization.

Alternatively, such a component (A1) can be produced by dissolving, in a polymerization solvent, a monomer from which the constitutional unit (a1) is derived and, as necessary, a monomer from which a constitutional unit (for example, the constitutional unit (a10)) other than the constitutional unit (a1) is derived, adding thereto the radical polymerization initiator described above to carry out polymerization, and then carrying out a deprotection reaction.

Further, a —C(CF₃)₂—OH group may be introduced into the terminal of the component (A1) during the polymerization using a chain transfer agent such as HS—CH₂—CH₂—CH₂—C(CF₃)₂—OH in combination. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw) (in terms of polystyrene according to gel permeation chromatography (GPC)) of the component (A1) is not particularly limited, but is preferably 1000 to 50000, more preferably 2000 to 30000, and still more preferably 3000 to 20000.

In a case where the Mw of the component (A1) is equal to or less than the preferable upper limits of the range, the resist composition exhibits a satisfactory solubility in a resist solvent for a resist enough to be used as a resist. On the contrary, in a case where the Mw of the component (A1) is equal to or greater than the preferable lower limits of the range, the dry etching resistance and the cross-sectional shape of the resist pattern are excellent.

Further, the dispersity (Mw/Mn) of the component (A1) is not particularly limited, but is preferably 1.0 to 4.0, more preferably 1.0 to 3.0, and particularly preferably 1.0 to 2.0. Further, Mn represents the number average molecular weight.

### In Regard to Component (A2)

In the resist composition of the present embodiment, a base material component (hereinafter, also referred to as “component (A2)”) which does not correspond to the component (A1) and whose solubility in a developing solution is changed by the action of an acid may be used in combination as the component (A).

The component (A2) is not particularly limited and may be optionally selected from a plurality of components of the related art which have been known as base material components for a chemically amplified resist composition and used.

As the component (A2), a polymer compound or a low-molecular-weight compound may be used alone or in combination of two or more kinds thereof.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist pattern having excellent various lithography characteristics such as high sensitivity, high resolution, and reduction of roughness is likely to be formed.

In the resist composition of the present embodiment, the content of the component (A) may be adjusted according to the thickness of the resist film intended to be formed.

### <Compound (D0)>

The compound (D0) (hereinafter, also referred to as “component (D0)”) is a compound represented by General Formula (d0). By containing the component (D0), in the resist composition of the present embodiment, the temporal stability of the resist composition during storage can be improved and the deterioration of the fine resolution of the resist composition after storage can be suppressed.

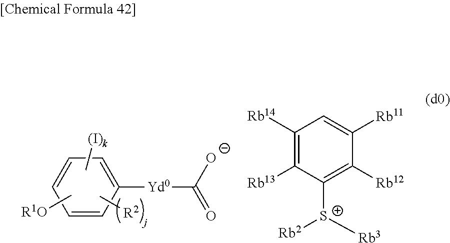

[In the formula, R¹ represents a saturated hydrocarbon group. I represents an iodine atom. R² represents a substituent. Yd⁰ represents a divalent linking group or a single bond. j represents an integer of 0 to 3. k represents an integer of 1 to 4. 1≤j+k≤4 is satisfied. Rb¹¹, Rb¹², Rb¹³, and Rb¹⁴ each independently represent a fluorine atom, a fluorinated alkyl group, or a hydrogen atom. At least one of Rb¹¹, Rb¹², Rb¹³ or Rb¹⁴ is a fluorine atom or a fluorinated alkyl group. Rb² and Rb³ each independently represent a hydrocarbon group which may have a substituent, or Rb² and Rb³ are bonded to each other to form a ring together with a sulfur atom in the formula.]

### (Component (D0) Anion Moiety)

In Formula (d0), R¹ represents a saturated hydrocarbon group. Examples of the saturated hydrocarbon group include a monovalent chain-like saturated hydrocarbon group having 1 to 10 carbon atoms and a monovalent aliphatic cyclic saturated hydrocarbon group having 3 to 20 carbon atoms.

Examples of the monovalent chain-like saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.0^{2,6}]decanyl group, a tricyclo[3.3.1.13,7]decanyl group, a tetracyclo[6.2.1.13,6.02,7]dodecanyl group, or an adamantyl group.

As the saturated hydrocarbon group as R¹, a monovalent chain-like saturated hydrocarbon group having 1 to 10 carbon atoms is preferable, a monovalent chain-like saturated hydrocarbon group having 1 to 6 carbon atoms is more preferable, a monovalent chain-like saturated hydrocarbon group having 1 to 3 carbon atoms is still more preferable, and a methyl group or an ethyl group is particularly preferable.

In Formula (d0), R² represents a substituent. Examples of the substituent as R² include an alkyl group, a nitro group, a chlorine atom, —C(═O)—O—Rd⁰¹, —O—C(═O)—Rd⁰², an amino group, and —NH—C(—O)—Rd⁰².

The alkyl group as a substituent as R² may be an alkyl group having 1 to 5 carbon atoms.

Rd⁰¹ represents an alkyl group, and may be a linear or branched alkyl group having 1 to 5 carbon atoms.

Rd⁰² represents a hydrocarbon group which may have a substituent.

In Formula (d0), j represents an integer of 0 to 3. From the viewpoint of solubility of the resist composition in a resist solvent, j represents preferably an integer of 0 to 2, more preferably 0 or 1, and still more preferably 0.

In a case where j represents an integer of 2 or greater, a plurality of R²'s may be the same as or different from each other.

In Formula (d0), k represents an integer of 1 to 4. From the viewpoint of achieving both improvement of sensitivity and the solubility of the resist composition in a resist solvent, k represents preferably an integer of 2 to 4, more preferably 2 or 3, and still more preferably 3.

In Formula (d0), Yd⁰ represents a single bond or a divalent linking group.

Examples of the divalent linking group as Yd⁰ include the same groups as those for the divalent linking group as Ya^{x1} in General Formula (a10-1).

As the divalent linking group as Yd⁰, —C(═O)—O—, —O—C(═O)—, —O—, —C(═O)—, —NH—, —S—, a linear or branched aliphatic hydrocarbon group, or a combination thereof is preferable. As the linear or branched aliphatic hydrocarbon group, a linear or branched alkylene group having 1 to 6 carbon atoms or a linear or branched alkenylene group having 2 to 6 carbon atoms is preferable. As the divalent linking group as Ld, —C(═O)—O—, —O—C(═O)—, —O—, or —C(═O)— is more preferable.

From the viewpoint of improving solubility in a developing solution, Yd⁰ represents preferably a single bond.

Specific examples of the anion moiety of the component (D0) are shown below.

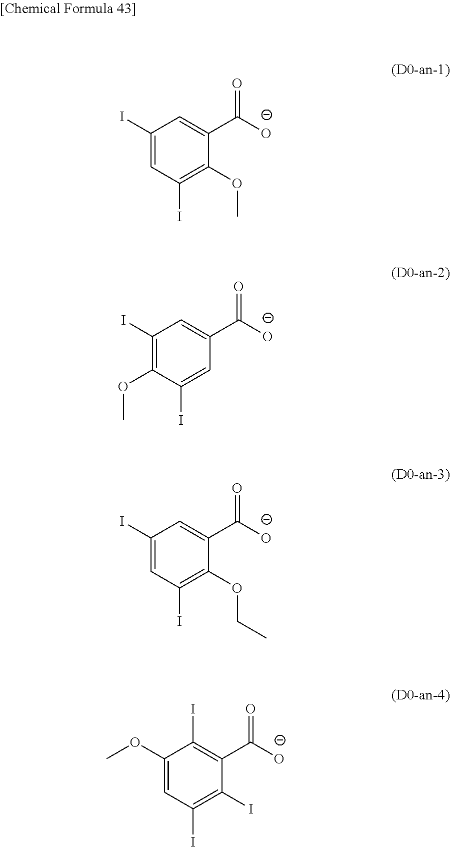

### (Cation Moiety of Component (D0))

In Formula (d0), Rb¹¹, Rb¹², Rb¹³, and Rb¹⁴ each independently represent a fluorine atom, a fluorinated alkyl group, or a hydrogen atom. At least one of Rb¹¹, Rb¹², Rb¹³, or Rb¹⁴ is a fluorine atom or a fluorinated alkyl group.

Examples of the fluorinated alkyl group include a fluorinated alkyl group having 1 to 5 carbon atoms, and a trifluoromethyl group is preferable.

Rb² and Rb³ each independently represent a hydrocarbon group which may have a substituent, or Rb² and Rb³ are bonded to each other to form a ring together with the sulfur atom in the formula.

In a case where Rb² or Rb³ is a hydrocarbon group which may have a substituent, examples of the hydrocarbon group as Rb² and Rb³ include a linear or branched alkyl group and a cyclic hydrocarbon group.

The linear alkyl group as Rb² and Rb³ may have, for example, 1 to 5 carbon atoms. Specific examples thereof include a methyl group, an ethyl group, an n-propyl group, an n-butyl group, and an n-pentyl group.

The branched alkyl group as Rb² and Rb³ may have, for example, 3 to 10 carbon atoms. Specific examples thereof include an isopropyl group, an isobutyl group, a tert-butyl group, an isopentyl group, a neopentyl group, a 1,1-diethylpropyl group, and a 2,2-dimethylbutyl group.

In a case where Rb² or Rb³ represents a cyclic hydrocarbon group, the hydrocarbon group may be an alicyclic hydrocarbon group or an aromatic hydrocarbon group and may be a polycyclic group or a monocyclic group.

The alicyclic hydrocarbon group which is a monocyclic group may be, for example, a group in which one hydrogen atom has been removed from a monocycloalkane. The monocycloalkane may have 3 to 6 carbon atoms, and specific examples thereof include cyclopentane and cyclohexane.

The alicyclic hydrocarbon group which is a polycyclic group may be, for example, a group in which one hydrogen atom has been removed from a polycycloalkane. The polycycloalkane may have 7 to 12 carbon atoms, and specific examples thereof include adamantane, norbornane, isobornane, tricyclo[5.2.1.0^{2,6}]decane, and tetracyclododecane.

Examples of the aromatic hydrocarbon group as Rb² or Rb³ include a group in which one or more hydrogen atoms have been removed from an aromatic hydrocarbon ring having 5 to 30 carbon atoms.

Among these, Rb² or Rb³ is preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent which may be included in the hydrocarbon group as Rb² or Rb³ include a methyl group, an ethyl group, a propyl group, a hydroxyl group, a carboxyl group, a halogen atom, an alkoxy group (such as a methoxy group, an ethoxy group, a propoxy group, or a butoxy group), and an alkyloxycarbonyl group.

Rb² and Rb³ may be the same as or different from each other.

As Rb² and Rb³, an aromatic hydrocarbon group is preferable, a phenyl group which may have a substituent is more preferable, and a group represented by Formula (D0-ca-s1) is still more preferable.

It is preferable that Rb² and Rb³ are bonded to each other to form a ring together with the sulfur atom in the formula.

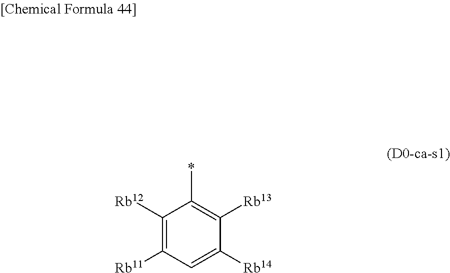

[In the formula, Rb¹¹, Rb¹², Rb¹³, and Rb¹⁴ each independently represent a fluorine atom, a fluorinated alkyl group, or a hydrogen atom. At least one of Rb¹¹, Rb¹², Rb¹³, or Rb¹⁴ is a fluorine atom or a fluorinated alkyl group. Here, * represents a bonding site to a sulfur atom.]

The number of all fluorine atoms included in Rb¹¹, Rb¹², Rb¹³, and Rb¹⁴ in General Formula (d0) is preferably 2 or greater and more preferably 3 or greater.

It is preferable that the cation moiety of General Formula (d0) has at least one phenyl group having two or more fluorine atoms.

Specific examples of the cation moiety of the component (D0) are shown below.

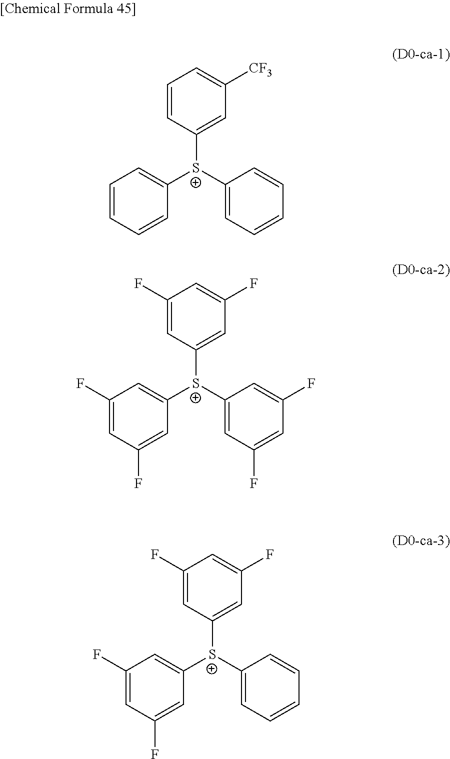 [SMILES: FC(F)(F)c1cccc([S+](c2ccccc2)c2ccccc2)c1 ; Fc1cc(F)cc([S+](c2cc(F)cc(F)c2)c2cc(F)cc(F)c2)c1 ; Fc1cc(F)cc([S+](c2ccccc2)c2cc(F)cc(F)c2)c1]

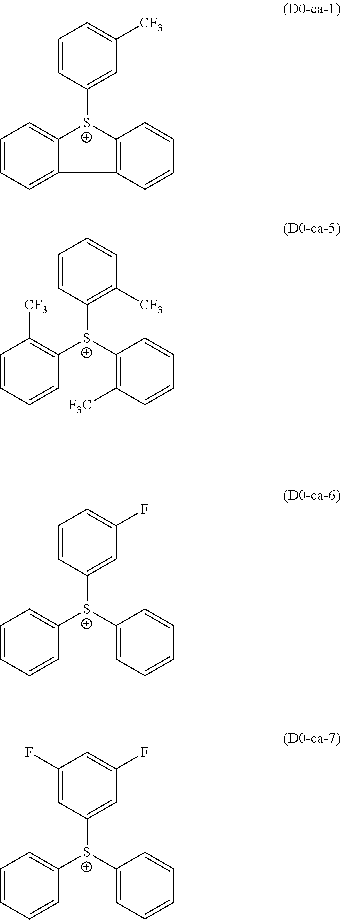 [SMILES: FC(F)(F)c1cccc(-[s+]2c3ccccc3c3ccccc32)c1 ; FC(F)(F)c1ccccc1[S+](c1ccccc1C(F)(F)F)c1ccccc1C(F)(F)F ; Fc1cccc([S+](c2ccccc2)c2ccccc2)c1 ; Fc1cc(F)cc([S+](c2ccccc2)c2ccccc2)c1]

Specific examples of the component (D0) are shown below.

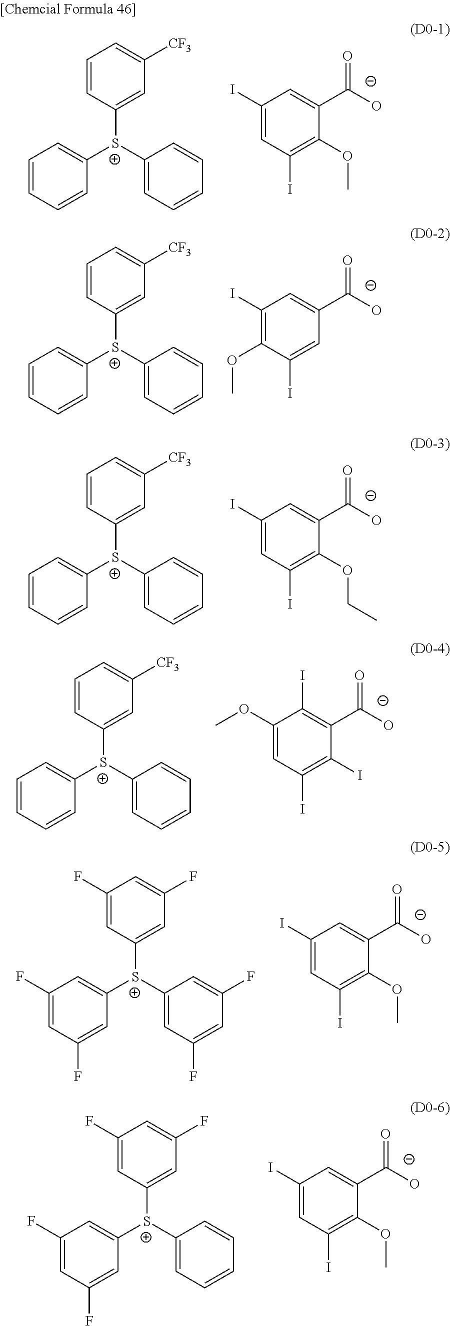

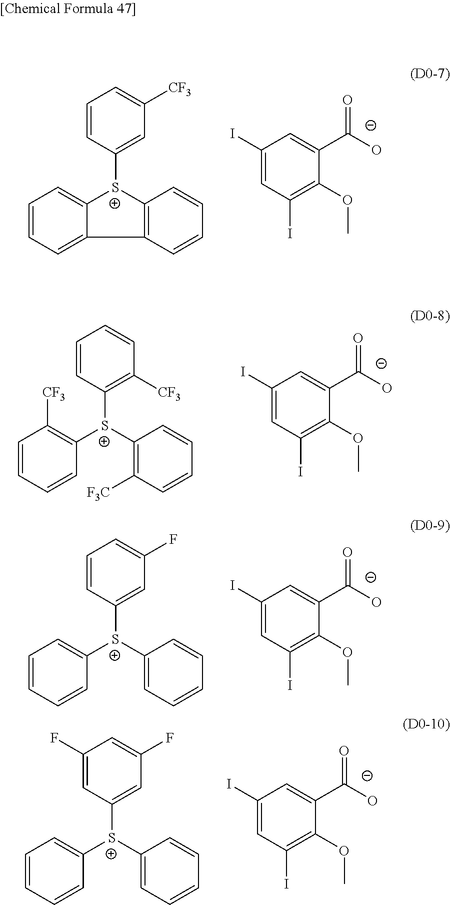

In the resist composition of the present embodiment, the component (D0) may be used alone or in combination of two or more kinds thereof.

In the resist composition of the present embodiment, the content of the component (D0) is preferably 1 to 40 parts by mass, more preferably 3 to 30 parts by mass, and still more preferably 5 to 20 parts by mass with respect to 100 parts by mass of the component (A).

In a case where the content of the component (D0) is equal to or greater than the lower limits of the above-described preferable range, the temporal stability of the resist composition during storage can be improved and the deterioration of the fine resolution of the resist composition after storage can be suppressed. Meanwhile, in a case where the content of the component (D0) is equal to or less than the upper limits of the above-described preferable ranges, the sensitivity is likely to be maintained satisfactorily, and the solubility in the developing solution is also likely to be improved.

### <Other Components>

The resist composition of the present embodiment may further contain other components in addition to the component (A) and the component (D0) described above. Examples of the other components include a component (B), a component (D) (excluding the component (D0)), a component (E), a component (F), and a component (S), which are described below.

### <<Acid Generator Component (B)>>

It is preferable that the resist composition of the present embodiment further contains an acid generator component (B) which generates an acid upon light exposure.

The component (B) is not particularly limited, and those which have been suggested so far as an acid generator for a chemically amplified resist composition can be used.

Examples of the acid generator include various acid generators, onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzyl sulfonate-based acid generators, imino sulfonate-based acid generators, and disulfone-based acid generators. The contained form of the component (B) may be a form of a compound, a form in which the component (B) is incorporated into the component (A1) as the constitutional unit (a5) described above, or both of these forms.

Examples of the onium salt-based acid generators include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

Examples of the onium salt-based acid generators include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

### [Chemical Formula 48]

[In the formulae, R¹⁰¹ and R¹⁰⁴ to R¹⁰⁸ each independently represent a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represent a single bond, an alkylene group, or a fluorinated alkylene group. Here, Y¹⁰¹ and V¹⁰¹ do not simultaneously form a single bond. L¹⁰¹ and L¹⁰² each independently represent a single bond or an oxygen atom. L¹⁰³ to L¹⁰⁵ each independently represent a single bond, —CO—, or —SO₂—. m represents an integer of 1 or greater, and M′^{m+} represents an m-valent onium cation.]

### {Anion Moiety}

Anion in Component (b-1)

In Formula (b-1), R¹⁰¹ represents a cyclic group which may have a substituent, a chain-like alkyl group which may have a substituent, or a chain-like alkenyl group which may have a substituent.

Cyclic group which may have substituent:

The cyclic group is preferably a cyclic hydrocarbon group, and the cyclic hydrocarbon group may be an aromatic hydrocarbon group or an aliphatic hydrocarbon group. The aliphatic hydrocarbon group means a hydrocarbon group that has no aromaticity. In addition, it is preferable that the aliphatic hydrocarbon group is saturated.

The aromatic hydrocarbon group as R¹⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R¹⁰¹ include benzene, fluorene, naphthalene, anthracene, phenanthrene, biphenyl, or an aromatic heterocyclic ring in which some carbon atoms constituting any of these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as R¹⁰¹ include a group in which one hydrogen atom has been removed from the aromatic ring (an aryl group such as a phenyl group or a naphthyl group) and a group in which one hydrogen atom of the aromatic ring is substituted with an alkylene group (for example, a benzyl group, a phenethyl group, or a 1-naphthylmethyl group). The alkylene group (an alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R¹⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable. As the polycycloalkane, a group having 7 to 30 carbon atoms is preferable. Among these, a polycycloalkane having a crosslinked ring polycyclic skeleton such as adamantane, norbornane, isobornane, tricyclo[5.2.1.0^{2,6}]decane, or tetracyclododecane; and a polycycloalkane having a condensed ring polycyclic skeleton such as a cyclic group having a steroid skeleton are more preferable as the polycycloalkane.

Among these, as the cyclic aliphatic hydrocarbon group as R¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is still more preferable, and an adamantyl group is particularly preferable.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable. Specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R¹⁰¹ may have a heteroatom such as a heterocyclic ring. Specific examples thereof include lactone-containing cyclic groups each represented by General Formulae (a2-r-1) to (a2-r-7), —SO₂-containing cyclic groups each represented by General Formulae (b5-r-1) to (b5-r-4), and other heterocyclic groups each represented by Chemical Formulae (r-hr-1) to (r-hr-16). In the formulae, * represents a bonding site bonded to Y¹⁰¹ in Formula (b-1).

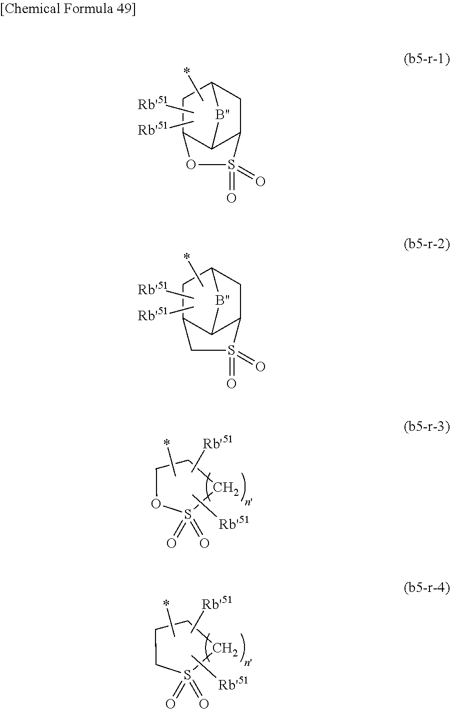

[In the formulae, Rb′51's each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, or an —SO₂-containing cyclic group; B″ represents an alkylene group having 1 to 5 carbon atoms which may have an oxygen atom or a sulfur atom, an oxygen atom, or a sulfur atom; and n′ represents an integer of 0 to 2. * represents a bonding site.]

In General Formulae (b5-r-1) and (b5-r-2), B″ represents an alkylene group having 1 to 5 carbon atoms which may have an oxygen atom or a sulfur atom, an oxygen atom, or a sulfur atom.

As B″, an alkylene group having 1 to 5 carbon atoms or —O— is preferable, an alkylene group having 1 to 5 carbon atoms is more preferable, and a methylene group is still more preferable.

In General Formulae (b5-r-1) to (b5-r-4), Rb′51's each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, and among these, it is preferable that Rb′51's each independently represent a hydrogen atom or a cyano group.

Specific examples of the groups each represented by General Formulae (b5-r-1) to (b5-r-4) are shown below. In the formulae, “Ac” represents an acetyl group.

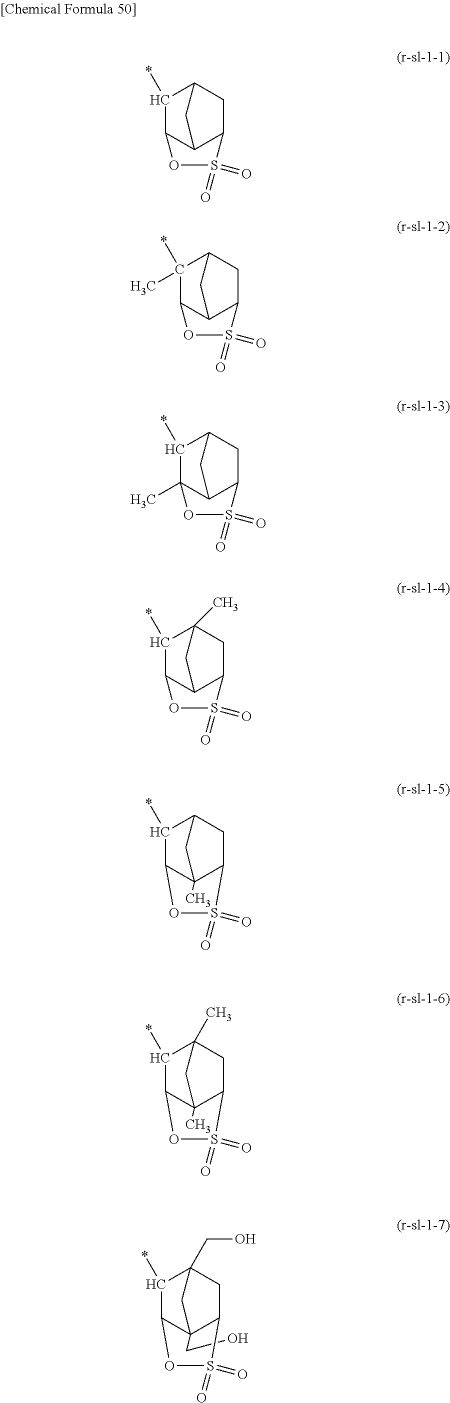

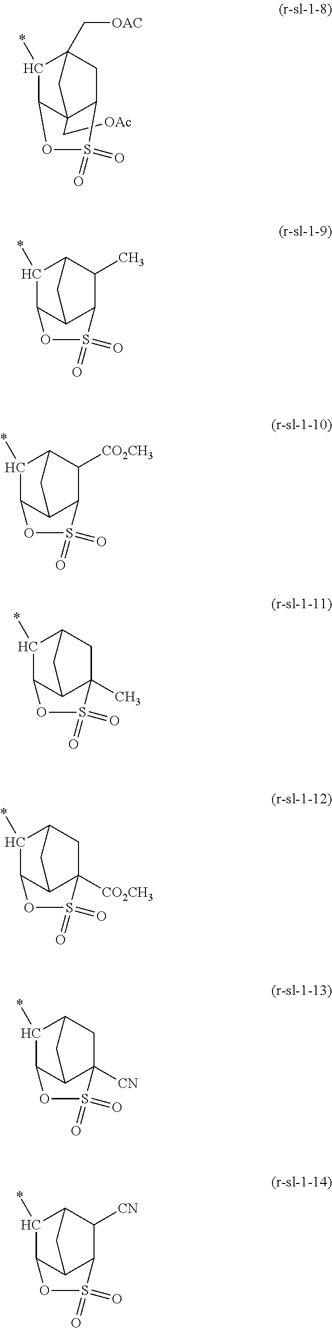

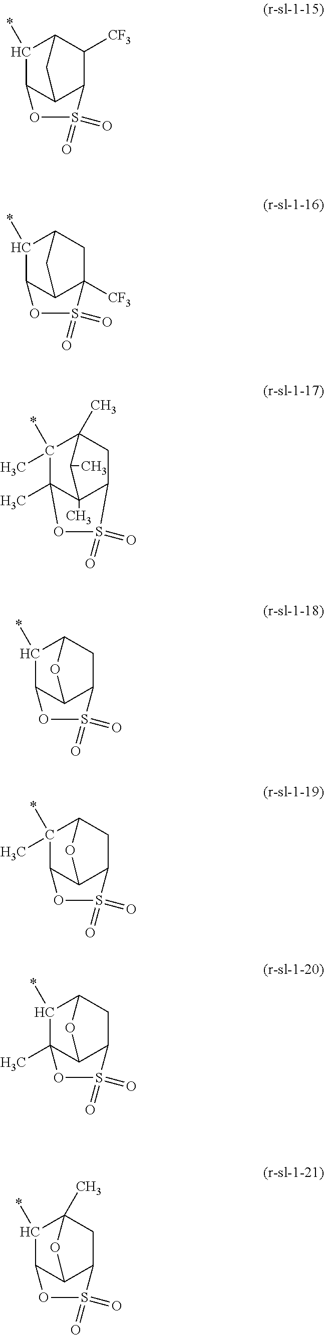

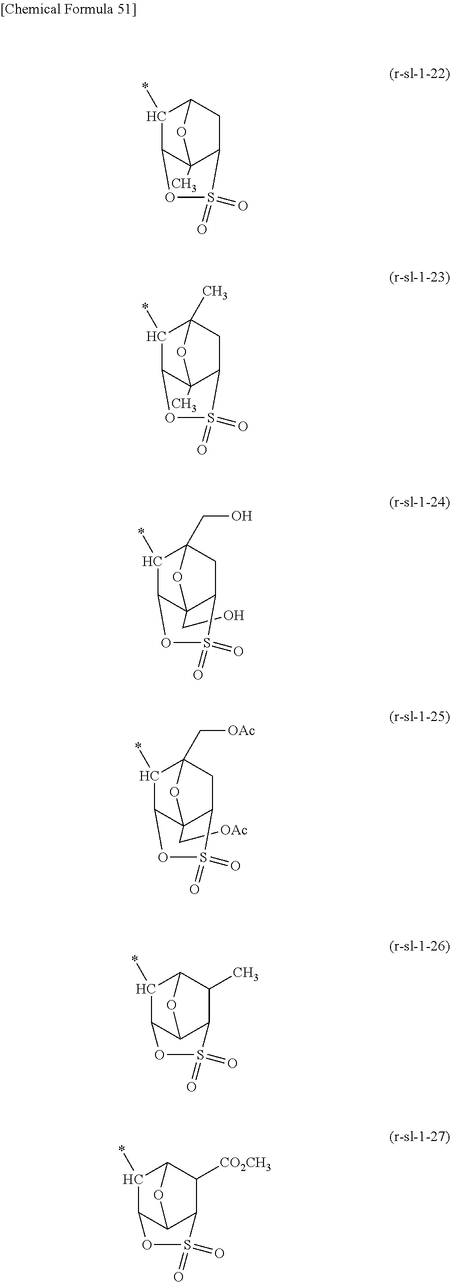

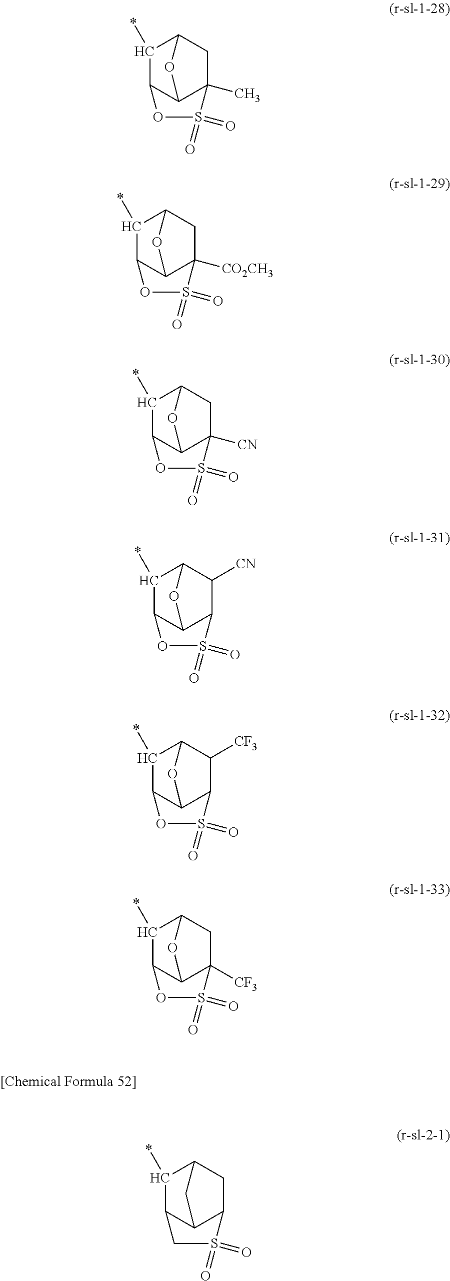

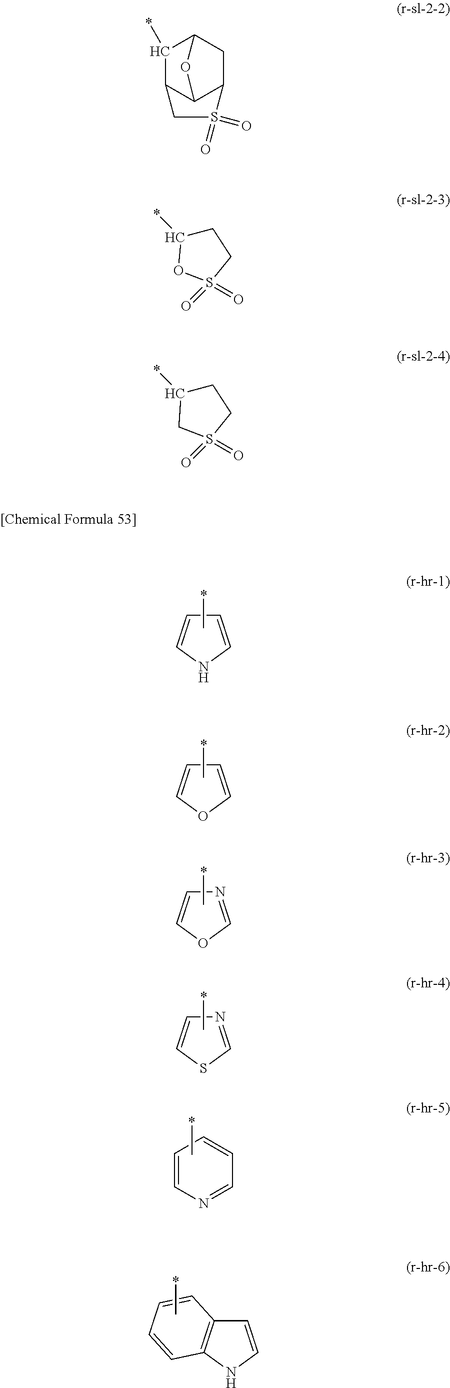

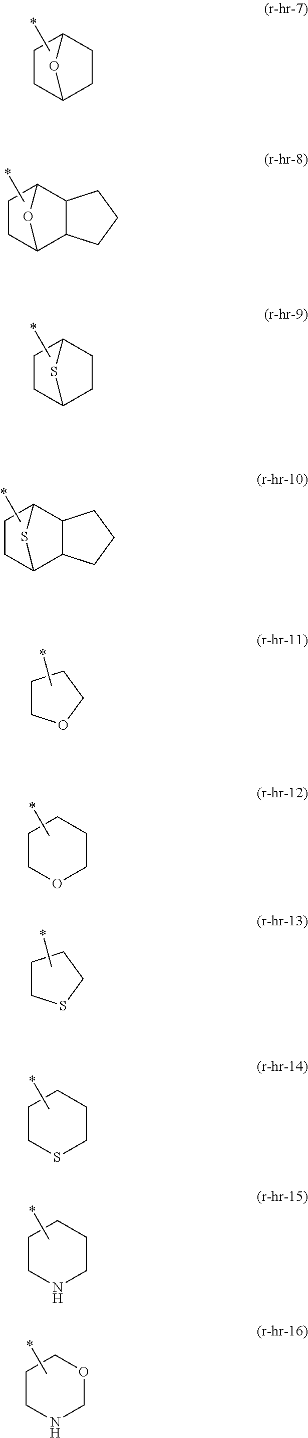

Examples of the substituent for the cyclic group as R¹⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as the substituent, a fluorine atom, a bromine atom, or an iodine atom is preferable.

Examples of the halogenated alkyl group as the substituent include a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

The cyclic hydrocarbon group as R¹⁰¹ may be a condensed cyclic group containing a condensed ring in which an aliphatic hydrocarbon ring and an aromatic ring are condensed. Examples of the condensed ring include a condensed ring in which one or more aromatic rings are condensed with a polycycloalkane having a crosslinked ring polycyclic skeleton. Specific examples of the crosslinked ring polycycloalkane include a bicycloalkane such as bicyclo[2.2.1]heptane (norbornane) and bicyclo[2.2.2]octane. As the condensed cyclic group, a group having a condensed ring in which two or three aromatic rings are condensed with a bicycloalkane is preferable, and a group having a condensed ring in which two or three aromatic rings are condensed with bicyclo[2.2.2]octane is more preferable. Specific examples of the condensed cyclic group as R¹⁰¹ include groups represented by Formulae (r-br-1) and (r-br-2). In the formulae, * represents a bonding site bonded to Y¹⁰¹ in Formula (b-1).

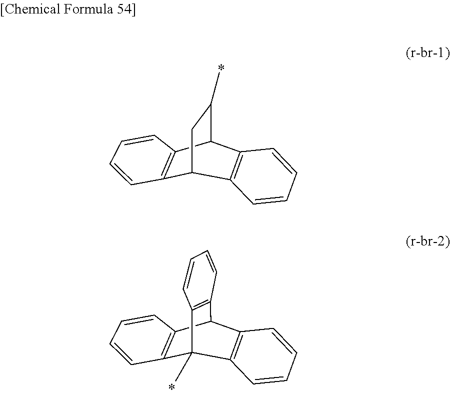

Examples of the substituent that the condensed cyclic group as R¹⁰¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the condensed cyclic group include the same groups as those for the substituent of the cyclic group as R¹⁰¹.

Examples of the aromatic hydrocarbon group as the substituent of the condensed cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and heterocyclic groups each represented by Formulae (r-hr-1) to (r-hr-6).

Examples of the alicyclic hydrocarbon group as the substituent of the condensed cyclic group include a group in which one hydrogen atom has been removed from a monocycloalkane such as cyclopentane or cyclohexane; a group in which one hydrogen atom has been removed from a polycycloalkane such as adamantane, norbornane, isobornane, tricyclo[5.2.1.0^{2,6}]decane, or tetracyclododecane; lactone-containing cyclic groups each represented by General Formulae (a2-r-1) to (a2-r-7); —SO₂-containing cyclic groups each represented by General Formulae (b5-r-1) to (b5-r-4); and heterocyclic groups each represented by Formulae (r-hr-7) to (r-hr-16).

Chain-like alkyl group which may have substituent:

The chain-like alkyl group as R¹⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-like alkenyl group which may have substituent:

The chain-like alkenyl group as R¹⁰¹ may be linear or branched, and has preferably 2 to 10 carbon atoms, more preferably 2 to 5 carbon atoms, still more preferably 2 to 4 carbon atoms, and particularly preferably 3 carbon atoms. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among these, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹⁰¹.

In Formula (b-1), Y¹⁰¹ represents a single bond or a divalent linking group having an oxygen atom.

In a case where Y¹⁰¹ represents a divalent linking group having an oxygen atom, Y¹⁰¹ may have an atom other than the oxygen atom. Examples of the atom other than the oxygen atom include a carbon atom, a hydrogen atom, a sulfur atom, and a nitrogen atom.

Examples of the divalent linking group having an oxygen atom include linking groups each represented by General Formulae (y-a1-1) to (y-a1-7). In General Formulae (y-a1-1) to (y-a1-7), V′¹⁰¹ in General Formulae (y-a1-1) to (y-a1-7) is bonded to R¹⁰¹ in Formula (b-1).

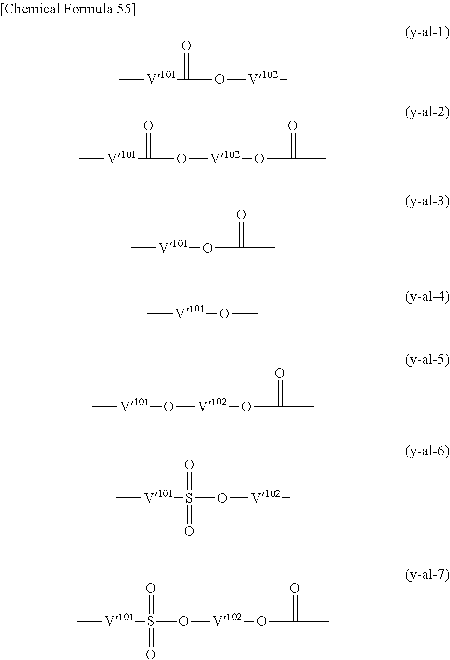

[In the formulae, V′¹⁰¹ represents a single bond or an alkylene group having 1 to 5 carbon atoms, and V′¹⁰² represents a divalent saturated hydrocarbon group having 1 to 30 carbon atoms.]

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

Further, a part of methylene group in the alkylene group as V′¹⁰¹ and V′¹⁰² may be substituted with a divalent aliphatic cyclic group having 5 to 10 carbon atoms. As the aliphatic cyclic group, a divalent group in which one hydrogen atom has been further removed from the cyclic aliphatic hydrocarbon group (a monocyclic aliphatic hydrocarbon group or a polycyclic aliphatic hydrocarbon group) as Ra′³ in Formula (a1-r-1) is preferable, and a cyclohexylene group, a 1,5-adamantylene group, or a 2,6-adamantylene group is more preferable.

In Formula (b-1), V¹⁰¹ represents a single bond, an alkylene group, or a fluorinated alkylene group. Among these, it is preferable that V¹⁰¹ represents a single bond or a linear fluorinated alkylene group having 1 to 4 carbon atoms.

In Formula (b-1), R¹⁰² represents a fluorine atom or a fluorinated alkyl group having 1 to 5 carbon atoms. R¹⁰² represents preferably a fluorine atom or a perfluoroalkyl group having 1 to 5 carbon atoms and more preferably a fluorine atom.

In a case where Y¹⁰¹ represents a single bond, specific examples of the anion moiety represented by Formula (b-1) include a fluorinated alkylsulfonate anion such as a trifluoromethanesulfonate anion or a perfluorobutanesulfonate anion. Further, in a case where Y¹⁰¹ represents a divalent linking group having an oxygen atom, specific examples thereof include an anion represented by any of Formulae (an-1) to (an-3).

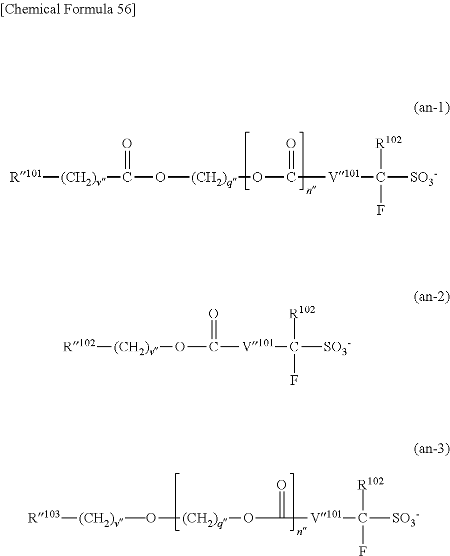

[In the formulae, R″¹⁰¹ represents an aliphatic cyclic group which may have a substituent, monovalent heterocyclic groups each represented by Chemical Formulae (r-hr-1) to (r-hr-6), a condensed cyclic group represented by Formula (r-br-1) or (r-br-2), a chain-like alkyl group which may have a substituent, or an aromatic cyclic group which may have a substituent. R″¹⁰² represents an aliphatic cyclic group which may have a substituent, a condensed cyclic group represented by Formula (r-br-1) or (r-br-2), lactone-containing cyclic groups each represented by General Formulae (a2-r-1) and (a2-r-3) to (a2-r-7), or —SO₂-containing cyclic groups each represented by General Formulae (b5-r-1) to (b5-r-4). R″¹⁰³ represents an aromatic cyclic group which may have a substituent, an aliphatic cyclic group which may have a substituent, or a chain-like alkenyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represent an integer of 0 to 3, each q″ independently represent an integer of 0 to 20, and n″ represents 0 or 1.]

As the aliphatic cyclic group as R″¹⁰¹, R″¹⁰², and R″¹⁰³ which may have a substituent, the groups described as the cyclic aliphatic hydrocarbon group as R¹⁰¹ in Formula (b-1) are preferable. Examples of the substituent include the same groups as those for the substituent which may substitute the cyclic aliphatic hydrocarbon group as R¹⁰¹ in Formula (b-1).

As the aromatic cyclic group as R″¹⁰¹ and R″¹⁰³ which may have a substituent, the groups described as the aromatic hydrocarbon group in the cyclic hydrocarbon group as R¹⁰¹ in Formula (b-1) are preferable. Examples of the substituent include the same groups as those for the substituent which may substitute the aromatic hydrocarbon group as R¹⁰¹ in Formula (b-1).

As the chain-like alkyl group as R″¹⁰¹ which may have a substituent, the groups described as the chain-like alkyl group as R¹⁰¹ in Formula (b-1) are preferable.

As the chain-like alkenyl group as R″¹⁰³ which may have a substituent, the groups described as the chain-like alkenyl group as R¹⁰¹ in Formula (b-1) are preferable.

Anion in Component (b-2)

In Formula (b-2), R¹⁰⁴ and R¹⁰⁵ each independently represent a cyclic group which may have a substituent, a chain-like alkyl group which may have a substituent, or a chain-like alkenyl group which may have a substituent, and examples thereof include the same groups as those for R¹⁰¹ in Formula (b-1). Here, R¹⁰⁴ and R¹⁰⁵ may be bonded to each other to form a ring.

As R¹⁰⁴ and R¹⁰⁵, a chain-like alkyl group which may have a substituent is preferable, and a linear or branched alkyl group or a linear or branched fluorinated alkyl group is more preferable.

The chain-like alkyl group has preferably 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R¹⁰⁵ decreases within the range of the number of carbon atoms from the viewpoint that the solubility in a solvent for a resist is also satisfactory. Further, in the chain-like alkyl group as R¹⁰⁴ and R¹⁰⁵, it is preferable that the number of hydrogen atoms substituted with fluorine atoms is as large as possible from the viewpoint that the acid strength increases and the transparency to high energy light with a wavelength of 250 nm or less or electron beams is improved. The proportion of fluorine atoms in the chain-like alkyl group, that is, the fluorination ratio is preferably 70% to 100% and more preferably 90% to 100%, and it is most preferable that the chain-like alkyl group is a perfluoroalkyl group in which all hydrogen atoms are substituted with fluorine atoms.

In Formula (b-2), V¹⁰² and V¹⁰³ each independently represent a single bond, an alkylene group, or a fluorinated alkylene group, and examples thereof include the same groups as those for V¹⁰¹ in Formula (b-1).

In Formula (b-2), L¹⁰¹ and L¹⁰² each independently represent a single bond or an oxygen atom.

Anion in Component (b-3)

In Formula (b-3), R¹⁰⁶ to R¹⁰⁸ each independently represent a cyclic group which may have a substituent, a chain-like alkyl group which may have a substituent, or a chain-like alkenyl group which may have a substituent, and examples thereof include the same groups as those for R¹⁰¹ in Formula (b-1).

In Formula (b-3), L¹⁰³ to L¹⁰⁵ each independently represent a single bond, —CO—, or —SO₂—.

Among these, as the anion moiety of the component (B), an anion of the component (b-1) is preferable, and an anion represented by Formula (an-1) is more preferable.

### {Cation Moiety}

In Formulae (b-1), (b-2), and (b-3), M′^{m+} represents an m-valent onium cation. Among these, a sulfonium cation and an iodonium cation are preferable.

m represents an integer of 1 or greater.

Preferred examples of the cation moiety ((M′^{m+})_{l/m}) include organic cations each represented by General Formulae (ca-1) to (ca-3).

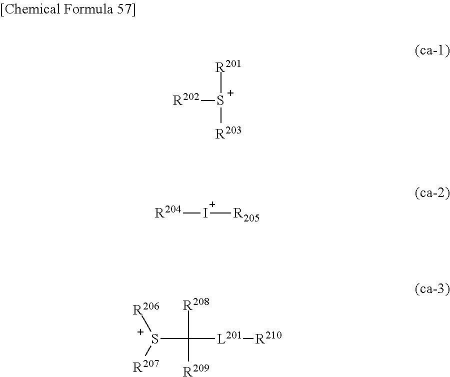

[In the formulae, R²⁰¹ to R²⁰⁷ each independently represent an aryl group, an alkyl group, or an alkenyl group, each of which may have a substituent. R²⁰¹ to R²⁰³ and R²⁰⁶ and R²⁰⁷ may be bonded to each other to form a ring together with the sulfur atoms in the formulae. R²⁰⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or a —SO₂-containing cyclic group which may have a substituent. L²⁰¹ represents —C(═O)— or —C(═O)—O—.]

In General Formulae (ca-1) to (ca-3), examples of the aryl group as R²⁰¹ to R²⁰⁷ include an unsubstituted aryl group having 6 to 20 carbon atoms, and a phenyl group or a naphthyl group is preferable.

As the alkyl group as R²⁰¹ to R²⁰⁷, a chain-like or cyclic alkyl group having 1 to 30 carbon atoms is preferable.

The alkenyl group as R²⁰¹ to R²⁰⁷ has preferably 2 to 10 carbon atoms.

Examples of the substituent which may be included in R²⁰¹ to R²⁰⁷ and R²¹⁰ include an alkyl group, a halogen atom, a halogenated alkyl group, a carbonyl group, a cyano group, an amino group, an aryl group, and groups each represented by General Formulae (ca-r-1) to (ca-r-7).

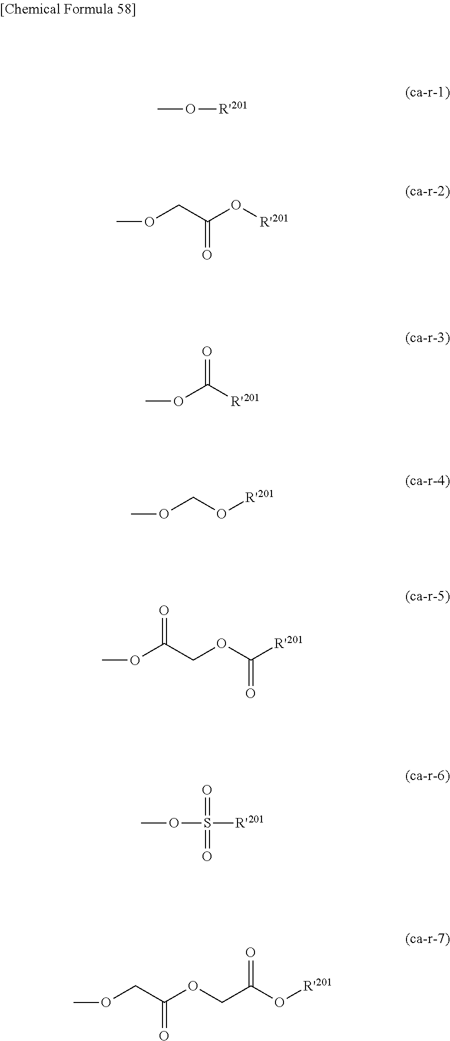

[In the formulae, R′²⁰¹'s each independently represent a hydrogen atom, a cyclic group which may have a substituent, a chain-like alkyl group which may have a substituent, or a chain-like alkenyl group which may have a substituent.]

Cyclic group which may have substituent:

The cyclic group is preferably a cyclic hydrocarbon group, and the cyclic hydrocarbon group may be an aromatic hydrocarbon group or an aliphatic hydrocarbon group. The aliphatic hydrocarbon group means a hydrocarbon group that has no aromaticity. Further, the aliphatic hydrocarbon group may be saturated or unsaturated. In general, it is preferable that the aliphatic hydrocarbon group is saturated.

The aromatic hydrocarbon group as R′²⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (an alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable. As the polycycloalkane, a group having 7 to 30 carbon atoms is preferable. Among these, a polycycloalkane having a crosslinked ring polycyclic skeleton such as adamantane, norbornane, isobornane, tricyclo[5.2.1.0^{2,6}]decane, or tetracyclododecane; and a polycycloalkane having a condensed ring polycyclic skeleton such as a cyclic group having a steroid skeleton are more preferable as the polycycloalkane.

Among these, as the cyclic aliphatic hydrocarbon group as R′²⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

As the branched aliphatic hydrocarbon group, a branched alkylene group is preferable. Specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R′²⁰¹ may have a heteroatom such as a heterocyclic ring. Specific examples thereof include lactone-containing cyclic groups each represented by General Formulae (a2-r-1) to (a2-r-7), —SO₂-containing cyclic groups each represented by General Formulae (b5-r-1) to (b5-r-4), and other heterocyclic groups each represented by Chemical Formulae (r-hr-1) to (r-hr-16).

Examples of the substituent for the cyclic group as R′²⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as the substituent, a fluorine atom is preferable.

Examples of the halogenated alkyl group as the substituent include a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

Chain-like alkyl group which may have substituent:

The chain-like alkyl group as R′²⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-like alkenyl group which may have substituent:

The chain-like alkenyl group as R′²⁰¹ may be linear or branched, and has preferably 2 to 10 carbon atoms, more preferably 2 to 5 carbon atoms, still more preferably 2 to 4 carbon atoms, and particularly preferably 3 carbon atoms. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among these, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

Examples of the cyclic group which may have a substituent, the chain-like alkyl group which may have a substituent, and the chain-like alkenyl group which may have a substituent as R′²⁰¹ include the same groups as those for the acid dissociable group represented by Formula (a1-r-2) which are the exemplary examples of the cyclic group which may have a substituent and the chain-like alkyl group which may have a substituent, in addition to those described above.

Among these, R′²⁰¹ represents preferably a cyclic group which may have a substituent, and more preferably a cyclic hydrocarbon group which may have a substituent. More specific preferred examples thereof include a phenyl group, a naphthyl group, a group in which one or more hydrogen atoms have been removed from a polycycloalkane, lactone-containing cyclic groups each represented by General Formulae (a2-r-1) to (a2-r-7), and —SO₂-containing cyclic groups each represented by General Formulae (b5-r-1) to (b5-r-4).

In General Formulae (ca-1) to (ca-3), in a case where R²⁰¹ to R²⁰³ and R²⁰⁶ and R²⁰⁷ are bonded to each other to form a ring together with a sulfur atom in the formula, these groups may be bonded to one another via a heteroatom such as a sulfur atom, an oxygen atom, or a nitrogen atom, or a functional group such as a carbonyl group, —SO—, —SO₂—, —SO₃—, —COO—, —CONH—, or —N(R_{N})— (here, R_{N} represents an alkyl group having 1 to 5 carbon atoms). As a ring to be formed, one ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represent a hydrogen atom or an alkyl group having 1 to 5 carbon atoms and are preferably a hydrogen atom or an alkyl group having 1 to 3 carbon atoms. In a case where R²⁰⁸ and R²⁰⁹ each independently represent an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or a —SO₂— containing cyclic group which may have a substituent.

Examples of the aryl group as R²¹⁰ include an unsubstituted aryl group having 6 to 20 carbon atoms, and a phenyl group or a naphthyl group is preferable.

As the alkyl group as R²¹⁰, a chain-like or cyclic alkyl group having 1 to 30 carbon atoms is preferable.

The alkenyl group as R²¹⁰ has preferably 2 to 10 carbon atoms.

As the-SO₂-containing cyclic group which may have a substituent as R²¹⁰, “—SO₂-containing polycyclic group” is preferable, and a group represented by General Formula (b5-r-1) is more preferable.

Specific examples of the cation represented by Formula (ca-1) are shown below.

Specific examples of the suitable cation represented by Formula (ca-1) include cations each represented by the following chemical formulae.

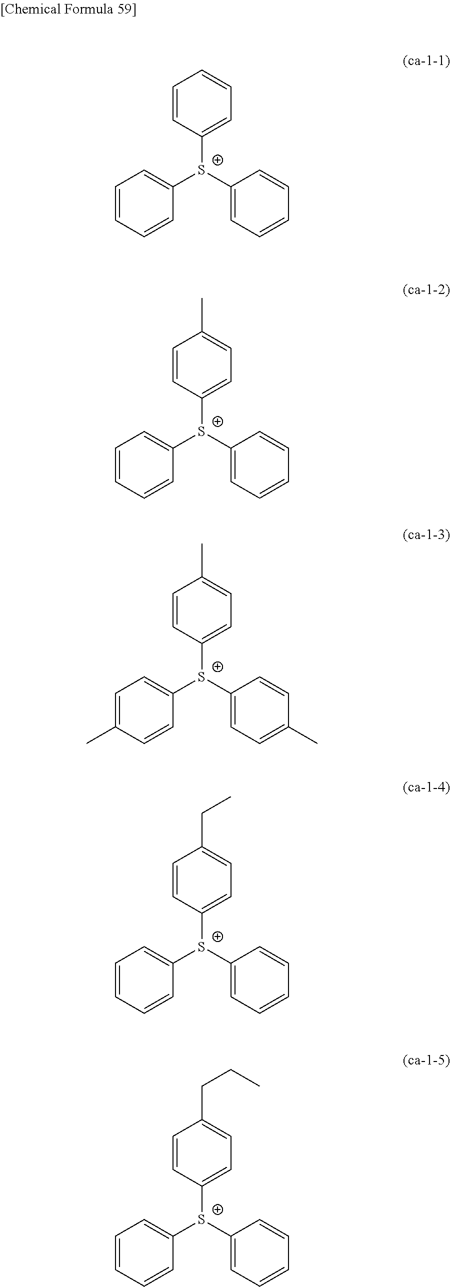

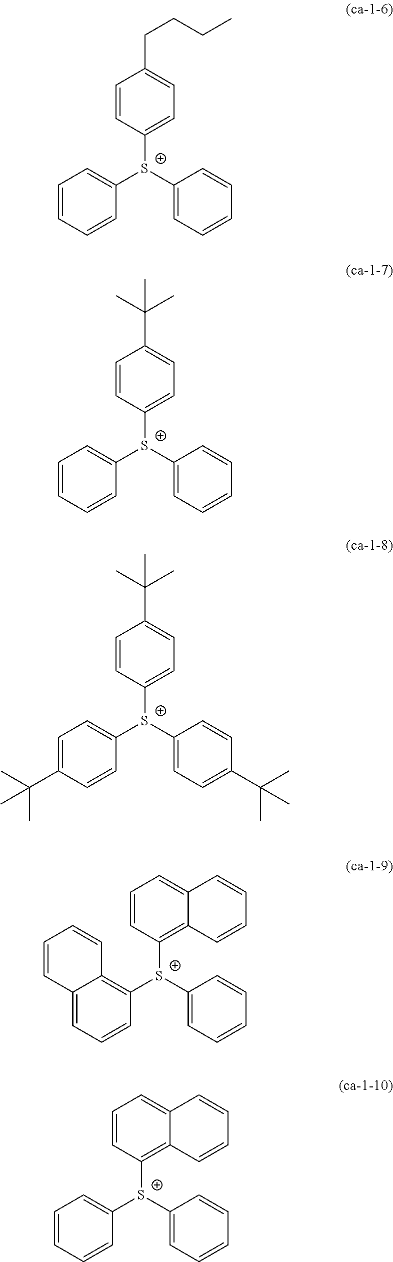 [SMILES: CCCCc1ccc([S+](c2ccccc2)c2ccccc2)cc1 ; CC(C)(C)c1ccc([S+](c2ccccc2)c2ccccc2)cc1 ; CC(C)(C)c1ccc([S+](c2ccc(C(C)(C)C)cc2)c2ccc(C(C)(C)C)cc2)cc1 ; c1ccc([S+](c2cccc3ccccc23)c2cccc3ccccc23)cc1 ; c1ccc([S+](c2ccccc2)c2cccc3ccccc23)cc1]

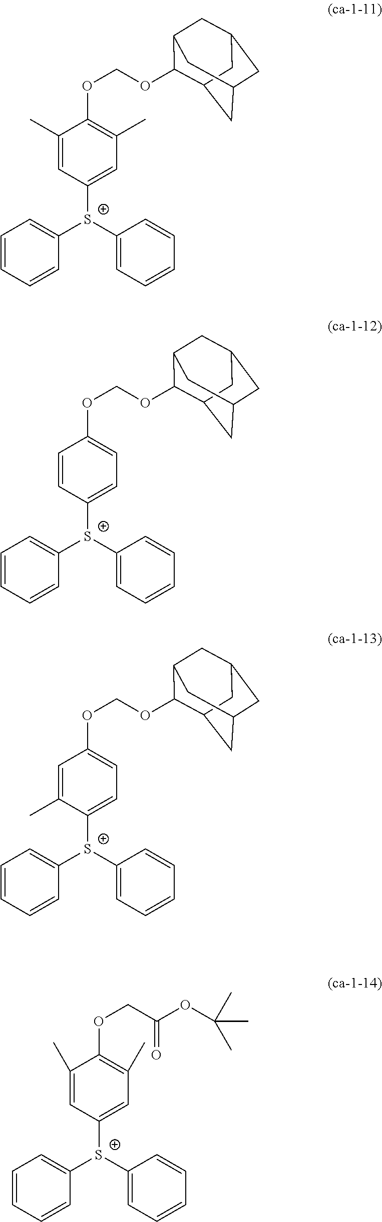 [SMILES: Cc1cc([S+](c2ccccc2)c2ccccc2)cc(C)c1OCOC1C2CC3CC(C2)CC1C3 ; c1ccc([S+](c2ccccc2)c2ccc(OCOC3C4CC5CC(C4)CC3C5)cc2)cc1 ; Cc1cc(OCOC2C3CC4CC(C3)CC2C4)ccc1[S+](c1ccccc1)c1ccccc1 ; Cc1cc([S+](c2ccccc2)c2ccccc2)cc(C)c1OCC(=O)OC(C)(C)C]

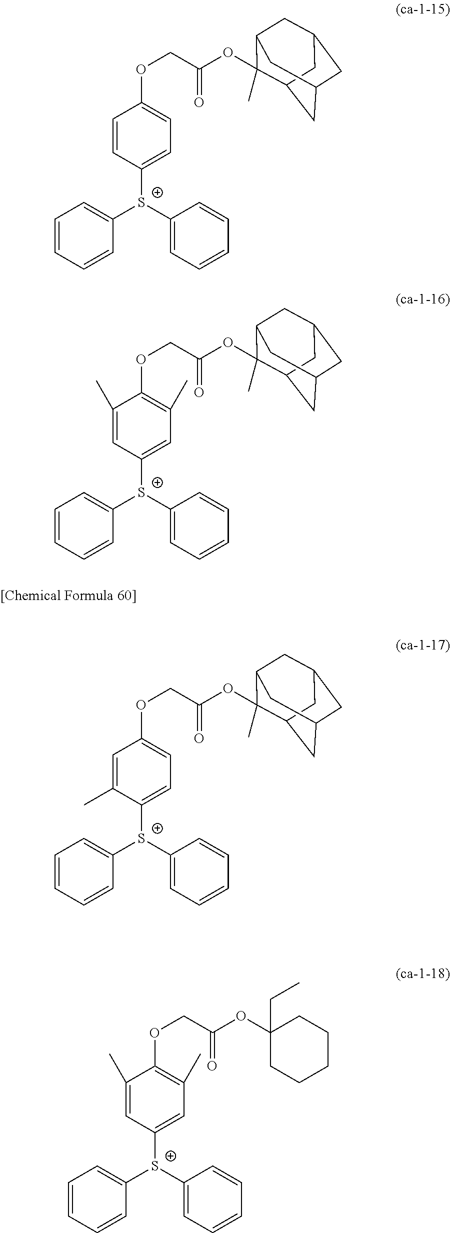 [SMILES: CC1(OC(=O)COc2ccc([S+](c3ccccc3)c3ccccc3)cc2)C2CC3CC(C2)CC1C3 ; Cc1cc([S+](c2ccccc2)c2ccccc2)cc(C)c1OCC(=O)OC1(C)C2CC3CC(C2)CC1C3 ; Cc1cc(OCC(=O)OC2(C)C3CC4CC(C3)CC2C4)ccc1[S+](c1ccccc1)c1ccccc1 ; CCC1(OC(=O)COc2c(C)cc([S+](c3ccccc3)c3ccccc3)cc2C)CCCCC1]

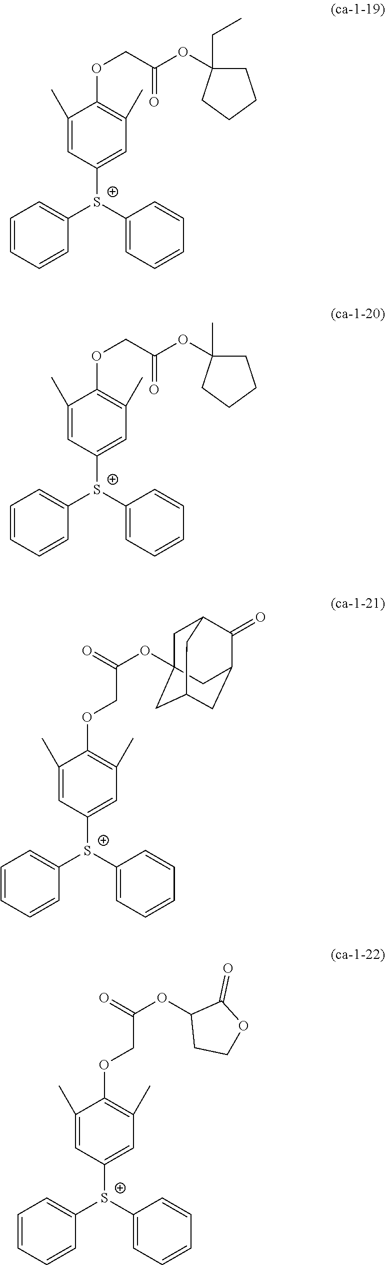 [SMILES: CCC1(OC(=O)COc2c(C)cc([S+](c3ccccc3)c3ccccc3)cc2C)CCCC1 ; Cc1cc([S+](c2ccccc2)c2ccccc2)cc(C)c1OCC(=O)OC1(C)CCCC1 ; Cc1cc([S+](c2ccccc2)c2ccccc2)cc(C)c1OCC(=O)OC12CC3CC(C1)C(=O)C(C3)C2 ; Cc1cc([S+](c2ccccc2)c2ccccc2)cc(C)c1OCC(=O)OC1CCOC1=O]

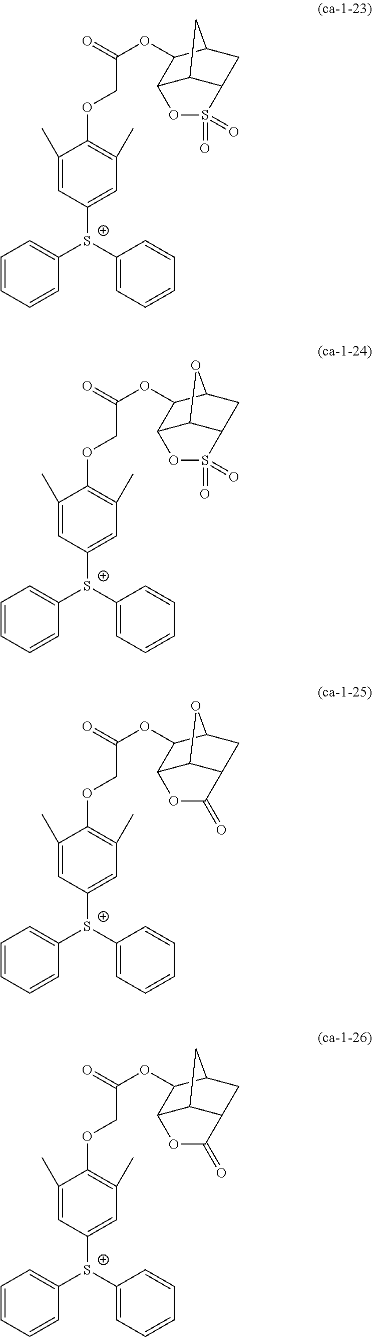 [SMILES: Cc1cc([S+](c2ccccc2)c2ccccc2)cc(C)c1OCC(=O)OC1C2CC3C1OS(=O)(=O)C3C2 ; Cc1cc([S+](c2ccccc2)c2ccccc2)cc(C)c1OCC(=O)OC1C2CC3C(O2)C1OS3(=O)=O ; Cc1cc([S+](c2ccccc2)c2ccccc2)cc(C)c1OCC(=O)OC1C2CC3C(=O)OC1C3O2 ; Cc1cc([S+](c2ccccc2)c2ccccc2)cc(C)c1OCC(=O)OC1C2CC3C(=O)OC1C3C2]

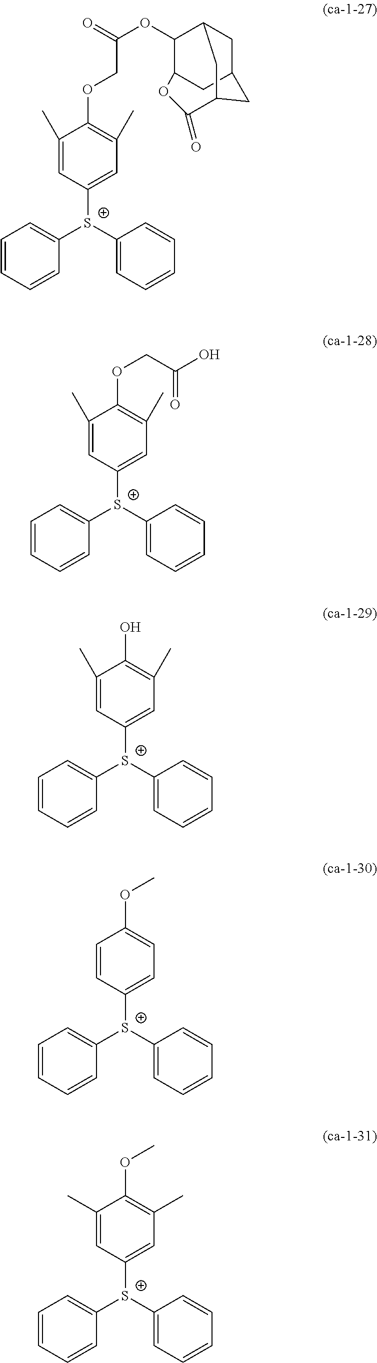 [SMILES: Cc1cc([S+](c2ccccc2)c2ccccc2)cc(C)c1OCC(=O)OC1C2CC3CC(C2)C(=O)OC1C3 ; Cc1cc([S+](c2ccccc2)c2ccccc2)cc(C)c1OCC(=O)O ; Cc1cc([S+](c2ccccc2)c2ccccc2)cc(C)c1O ; COc1ccc([S+](c2ccccc2)c2ccccc2)cc1 ; COc1c(C)cc([S+](c2ccccc2)c2ccccc2)cc1C]

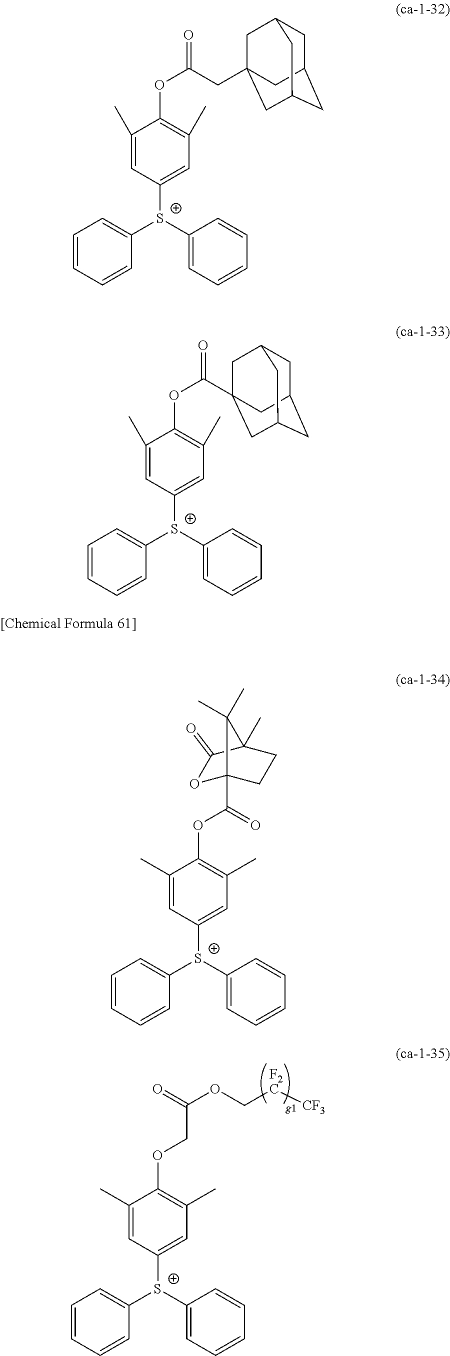

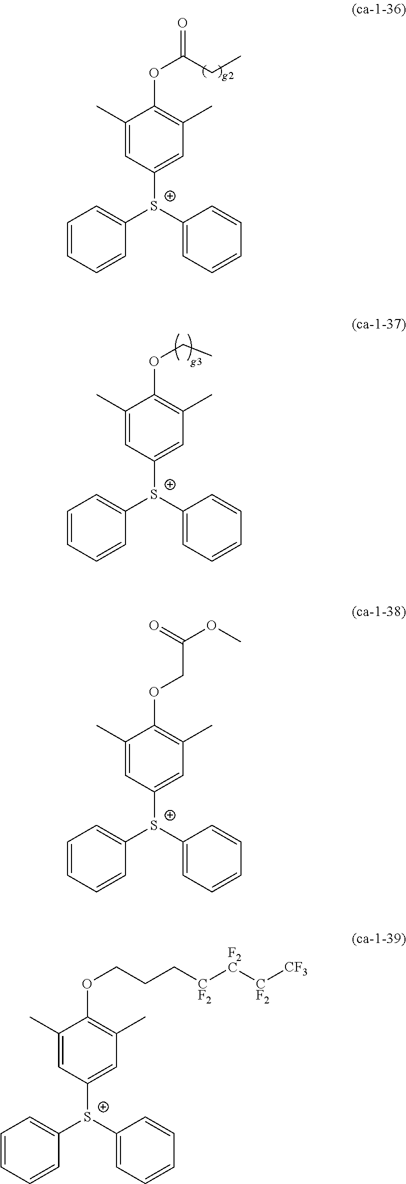

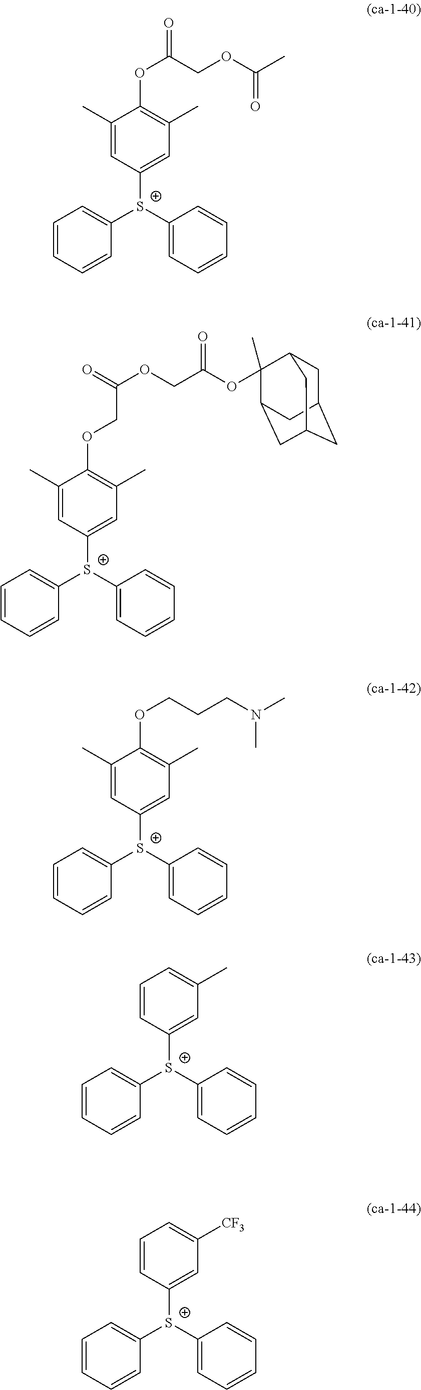 [SMILES: CC(=O)OCC(=O)Oc1c(C)cc([S+](c2ccccc2)c2ccccc2)cc1C ; Cc1cc([S+](c2ccccc2)c2ccccc2)cc(C)c1OCC(=O)OCC(=O)OC1(C)C2CC3CC(C2)CC1C3 ; Cc1cc([S+](c2ccccc2)c2ccccc2)cc(C)c1OCCCN(C)C ; Cc1cccc([S+](c2ccccc2)c2ccccc2)c1 ; FC(F)(F)c1cccc([S+](c2ccccc2)c2ccccc2)c1]

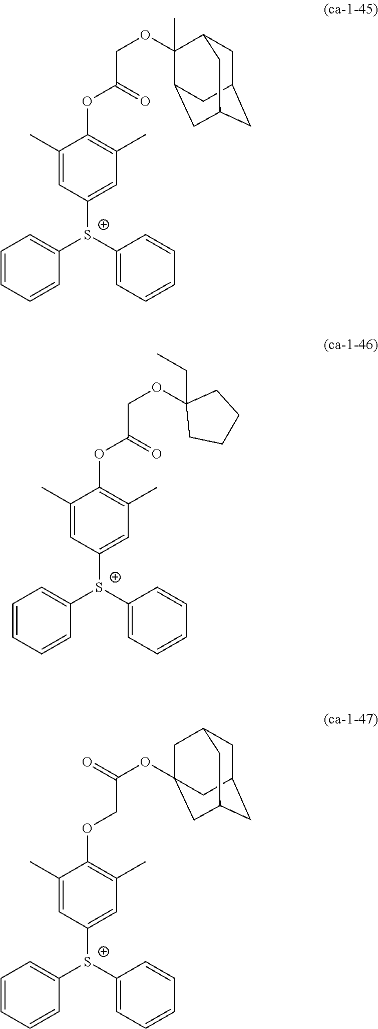 [SMILES: Cc1cc([S+](c2ccccc2)c2ccccc2)cc(C)c1OC(=O)COC1(C)C2CC3CC(C2)CC1C3 ; CCC1(OCC(=O)Oc2c(C)cc([S+](c3ccccc3)c3ccccc3)cc2C)CCCC1 ; Cc1cc([S+](c2ccccc2)c2ccccc2)cc(C)c1OCC(=O)OC12CC3CC(CC(C3)C1)C2]

[In the formulae, g1, g2, and g3 represent a repeating number, g1 represents an integer of 1 to 5, g2 represents an integer of 0 to 20, and g3 represents an integer of 0 to 20.]

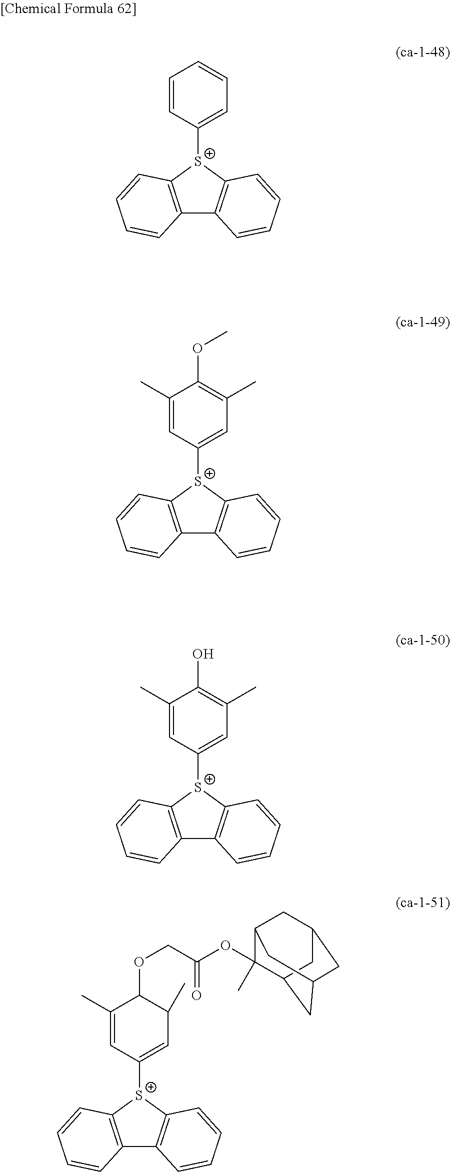 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; CC1=CC([s+]2c3ccccc3c3ccccc32)=CC(C)C1OCC(=O)OC1(C)C2CC3CC(C2)CC1C3]

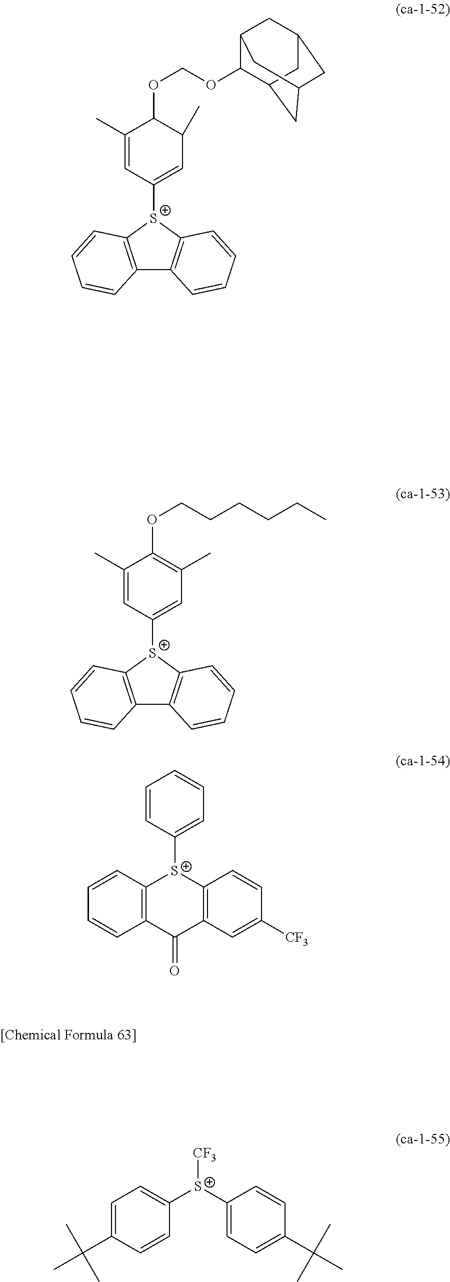

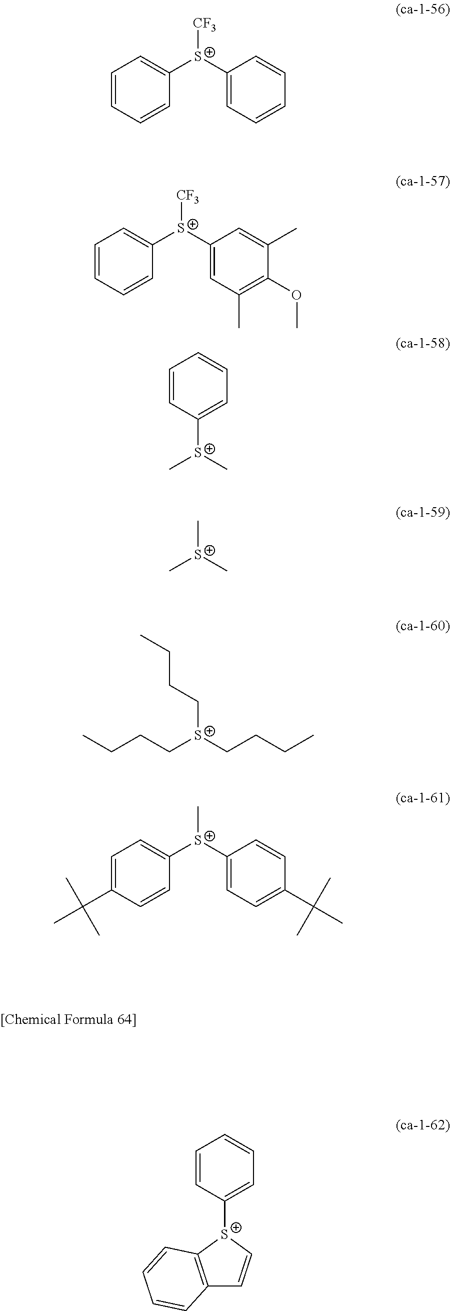

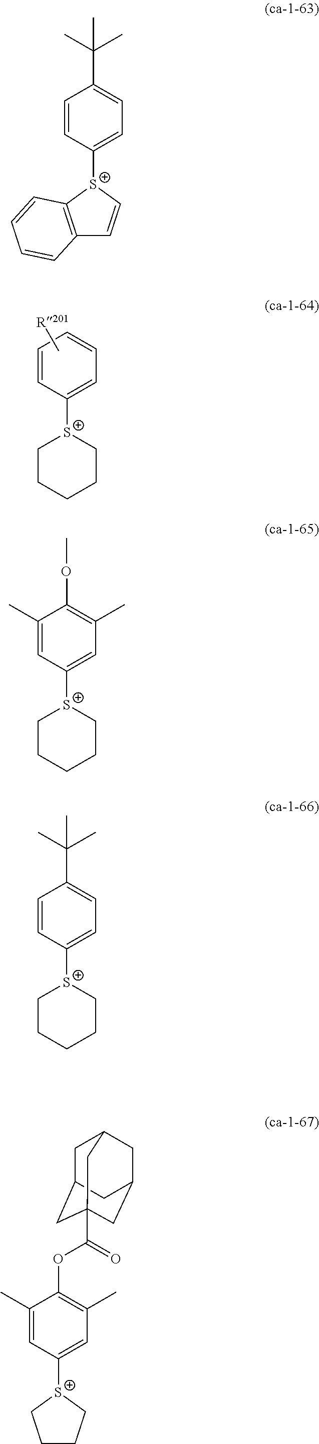

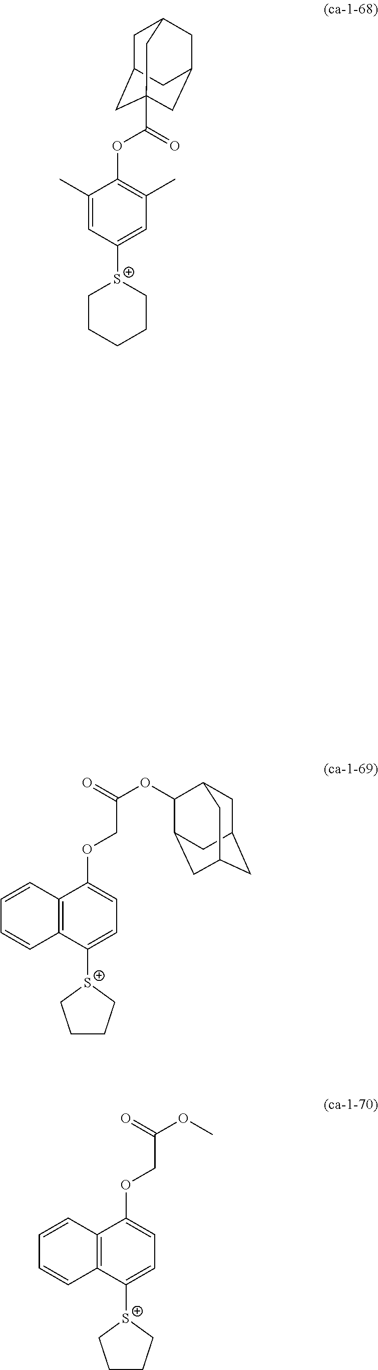 [SMILES: Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3 ; COC(=O)COc1ccc([S+]2CCCC2)c2ccccc12]

[In the formulae, R″²⁰¹ represents a hydrogen atom or a substituent, and examples of the substituent include the same groups as those for the substituents which may be included in R²⁰¹ to R²⁰⁷ and R²¹⁰ to R²¹².]

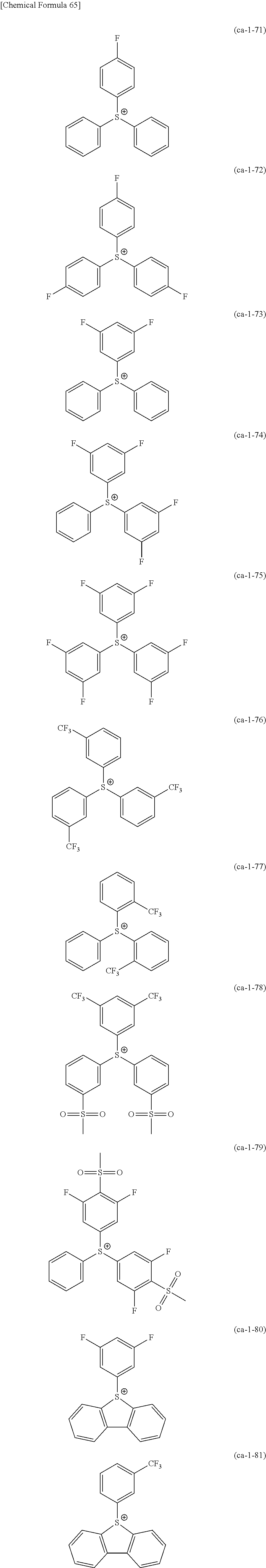

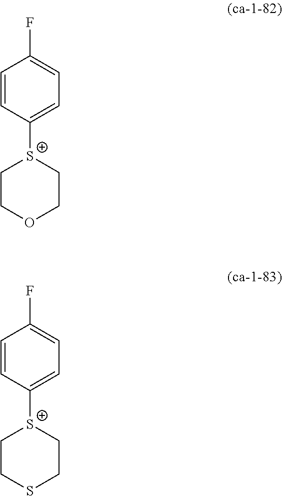 [SMILES: Fc1ccc([S+]2CCOCC2)cc1 ; Fc1ccc([S+]2CCSCC2)cc1]

Specific examples of the suitable cation represented by Formula (ca-2) include a diphenyliodonium cation and a bis(4-tert-butylphenyl) iodonium cation.

Specific examples of the suitable cation represented by Formula (ca-3) include cations each represented by Formulae (ca-3-1) to (ca-3-6).

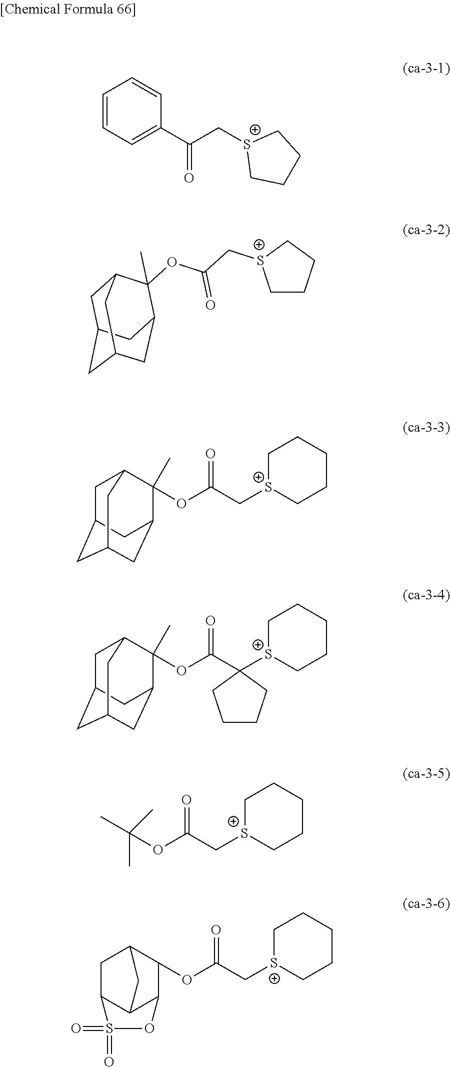 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC1C2CC3C1OS(=O)(=O)C3C2]

Among these, as the cation moiety of the component (B), a sulfonium cation is preferable, and a cation represented by Formula (ca-1) is more preferable.

In the resist composition of the present embodiment, the component (B) may be used alone or in combination of two or more kinds thereof.

In a case where the resist composition contains the component (B), the content of the component (B) in the resist composition is preferably less than 50 parts by mass, more preferably 10 to 40 parts by mass, and still more preferably 10 to 30 parts by mass with respect to 100 parts by mass of the component (A).

In a case where the content of the component (B) is set to be in the above-described preferable range, pattern formation can be sufficiently performed. Further, it is preferable that each component of the resist composition is dissolved in an organic solvent from the viewpoint that a uniform solution is easily obtained and the storage stability of the resist composition is enhanced.

### <<Base Component (D) (Excluding Component (D0))>>

The resist composition of the present embodiment may contain, in addition to the component (D0), a base component (hereinafter, also referred to as “component (D) (excluding the component (D0))”) that traps an acid generated upon light exposure (that is, controls the diffusion of the acid). The component (D) (excluding the component (D0)) acts as a quencher (acid diffusion control agent) that traps an acid generated upon light exposure in the resist composition.

Examples of the component (D) other than the component (D0) include a photodegradable base (D1) having an acid diffusion controllability (hereinafter, referred to as “component (D1)”) which is lost by the decomposition upon light exposure and a nitrogen-containing organic compound (D2) (hereinafter, referred to as “component (D2)”) which does not correspond to the component (D1).

Among these, the photodegradable base (the component (D1)) is preferable from the viewpoint of easily enhancing the roughness reducing property. Further, in a case where the component (D1) is contained, both the characteristics of improving the sensitivity and suppressing the occurrence of coating defects are likely to be enhanced.

The contained forms of the component (D1) and the component (D2) may be a form of a compound, a form in which the component (D1) and the component (D2) are incorporated into the component (A1) as the constitutional unit (a6) described above, or both of these forms. The compound described as the component (D1) below may be used as the acid generator component (component (B)) below depending on the combination with other compounds.

### In Regard to Component (D1)

The component (D) may contain a photodegradable base (D1) (hereinafter, referred to as “component (D1)”) which does not correspond to the component (D0).

In a case where a resist composition containing the component (D1) is obtained, the contrast between an exposed portion and an unexposed portion of the resist film can be further improved in a case of forming a resist pattern.

The component (D1) is not particularly limited as long as the component is decomposed upon light exposure and loses an acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable.

Since the components (d1-1) to (d1-3) are decomposed and lose the acid diffusion controllability (basicity), the components (d1-1) to (d1-3) do not act as a quencher at the exposed portion of the resist film, but act as a quencher at the unexposed portion of the resist film.

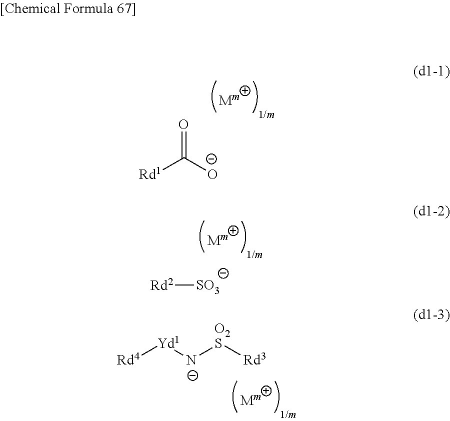

[In the formulae, Rd¹ to Rd⁴ represent a cyclic group which may have a substituent, a chain-like alkyl group which may have a substituent, or a chain-like alkenyl group which may have a substituent. Here, the carbon atom adjacent to the S atom as Rd² in Formula (d1-2) has no fluorine atom bonded thereto. Yd¹ represents a single bond or a divalent linking group. m represents an integer of 1 or greater, and M^{m+} 's each independently represent an m-valent organic cation.]

{Component (d1-1)}

### Anion Moiety

In Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹.

Among these, as Rd¹, represents an aromatic hydrocarbon group which may have a substituent, an aliphatic cyclic group which may have a substituent, or a chain-like alkyl group which may have a substituent is preferable. Examples of the substituent that may be included in these groups include a hydroxyl group, an oxo group, an alkyl group, an aryl group, a fluorine atom, a fluorinated alkyl group, lactone-containing cyclic groups each represented by General Formulae (a2-r-1) to (a2-r-7), an ether bond, an ester bond, and a combination thereof. In a case where an ether bond or an ester bond is included as the substituent, the substituent may be bonded through an alkylene group, and linking groups each represented by Formulae (y-a1-1) to (y-a1-5) are preferable as the substituent. Further, in a case where the aromatic hydrocarbon group, the aliphatic cyclic group, or the chain-like alkyl group as Rd¹ contains linking groups each represented by General Formulae (y-a1-1) to (y-a1-7) as a substituent, V′¹⁰¹ in General Formula (y-a1-1) to (y-a1-7) is bonded to the carbon atom constituting the aromatic hydrocarbon group, the aliphatic cyclic group, or the chain-like alkyl group as Rd¹ in General Formula (d3-1), in Formulae (y-a1-1) to (y-a1-7).

Suitable examples of the aromatic hydrocarbon group include a phenyl group, a naphthyl group, and a polycyclic structure having a bicyclooctane skeleton (a polycyclic structure consisting of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclo[5.2.1.0^{2,6}]decane, or tetracyclododecane is more preferable.

The chain-like alkyl group has preferably 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may contain an atom other than a fluorine atom. Examples of the atom other than a fluorine atom include an oxygen atom, a sulfur atom, and a nitrogen atom.

Specific preferred examples of the anion moiety in the component (d1-1) are shown below.

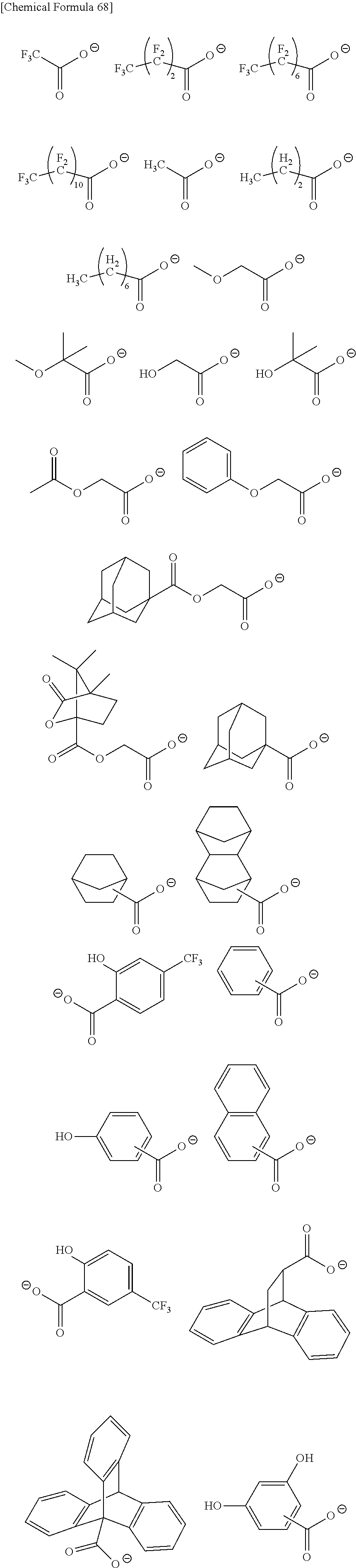

### Cation Moiety

In Formula (d1-1), M^{m+} represents an m-valent organic cation.

Suitable examples of the organic cation of M^{m+} include the same cations as those each represented by General Formulae (ca-1) to (ca-3), the cation represented by General Formula (ca-1) is more preferable, and cations each represented by Formulae (ca-1-1) to (ca-1-83) are still more preferable.

The component (d1-1) may be used alone or in combination of two or more kinds thereof.

The component (d1-1) may be used alone or in combination of two or more kinds thereof.

{Component (d1-2)}

### Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′²⁰¹.

Here, the carbon atom adjacent to the S atom as Rd² has no fluorine atom bonded thereto (the carbon atom is not substituted with fluorine). As a result, the anion of the component (d1-2) becomes an appropriately weak acid anion, thereby improving the quenching ability of the component (D).

As Rd², a chain-like alkyl group which may have a substituent or an aliphatic cyclic group which may have a substituent is preferable, and an aliphatic cyclic group which may have a substituent is more preferable.

The chain-like alkyl group has preferably 1 to 10 carbon atoms and more preferably 3 to 10 carbon atoms.

As the aliphatic cyclic group, a group (which may have a substituent) in which one or more hydrogen atoms have been removed from adamantane, norbornane, isobornane, tricyclo[5.2.1.0^{2,6}]decane, or tetracyclododecane; and a group in which one or more hydrogen atoms have been removed from camphor is more preferable.

The hydrocarbon group as Rd² may have a substituent, and examples of the substituent include the same groups as those for the substituent which may be included in the hydrocarbon group (an aromatic hydrocarbon group, an aliphatic cyclic group, or a chain-like alkyl group) as Rd¹ in Formula (d1-1).

Specific preferred examples of the anion moiety in the component (d1-2) are shown below.

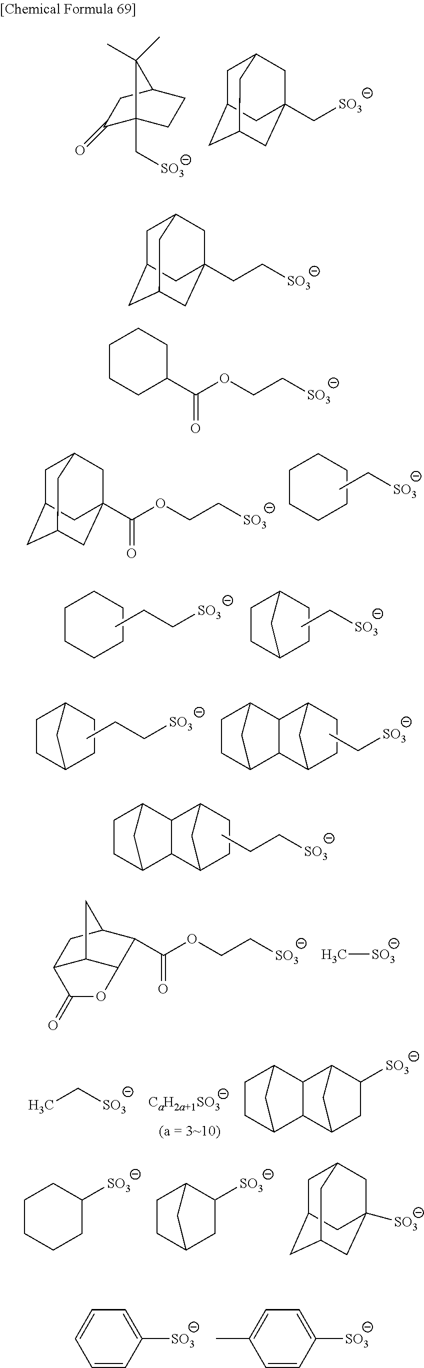

### Cation Moiety

In Formula (d1-2), M^{m+} represents an m-valent organic cation and is the same as M^{m+} in Formula (d1-1).

The component (d1-2) may be used alone or in combination of two or more kinds thereof.

{Component (d1-3)}

### Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group as Rd¹ are more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for R′²⁰¹.

Among these, an alkyl group which may have a substituent, an alkoxy group which may have a substituent, an alkenyl group which may have a substituent, or a cyclic group which may have a substituent is preferable.

It is preferable that the alkyl group as Rd⁴ is a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ is an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may have an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclo[5.2.1.0^{2,6}]decane, or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are enhanced. Further, in a case where Rd⁴ represents an aromatic group, the resist composition has excellent light absorption efficiency in lithography using EUV or the like as an exposure light source, and thus the sensitivity and lithography characteristics are enhanced.

In Formula (d1-3), Yd¹ represents a single bond or a divalent linking group.

The divalent linking group as Yd¹ is not particularly limited, and examples thereof include a divalent hydrocarbon group (an aliphatic hydrocarbon group or an aromatic hydrocarbon group) which may have a substituent and a divalent linking group having a heteroatom. These divalent linking groups are the same as those described above as the divalent hydrocarbon group which may have a substituent and the divalent linking group having a heteroatom described above as the divalent linking group as Ya²¹ in Formula (a2-1).

As Yd¹, a carbonyl group, an ester bond, an amide bond, an alkylene group, or a combination thereof is preferable. As the alkylene group, a linear or branched alkylene group is more preferable, and a methylene group or an ethylene group is still more preferable.

Specific preferred examples of the anion moiety in the component (d1-3) are shown below.

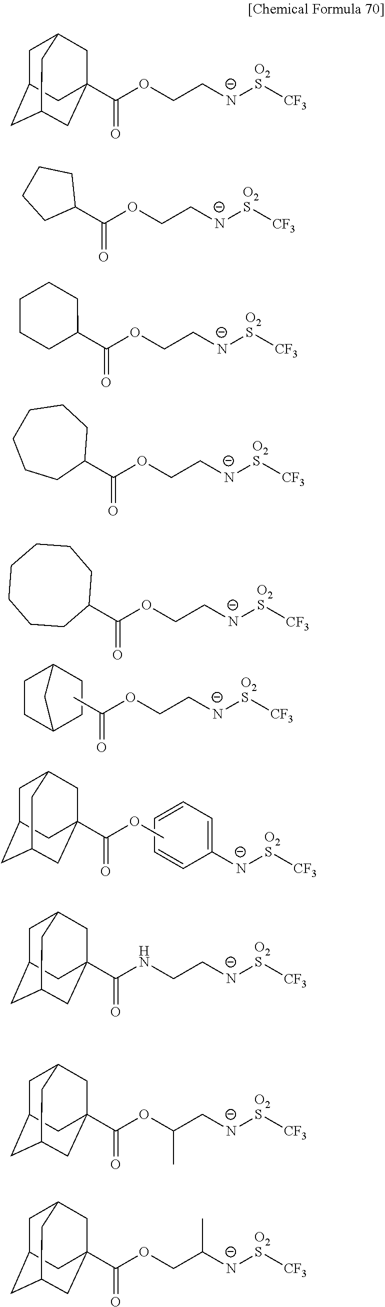

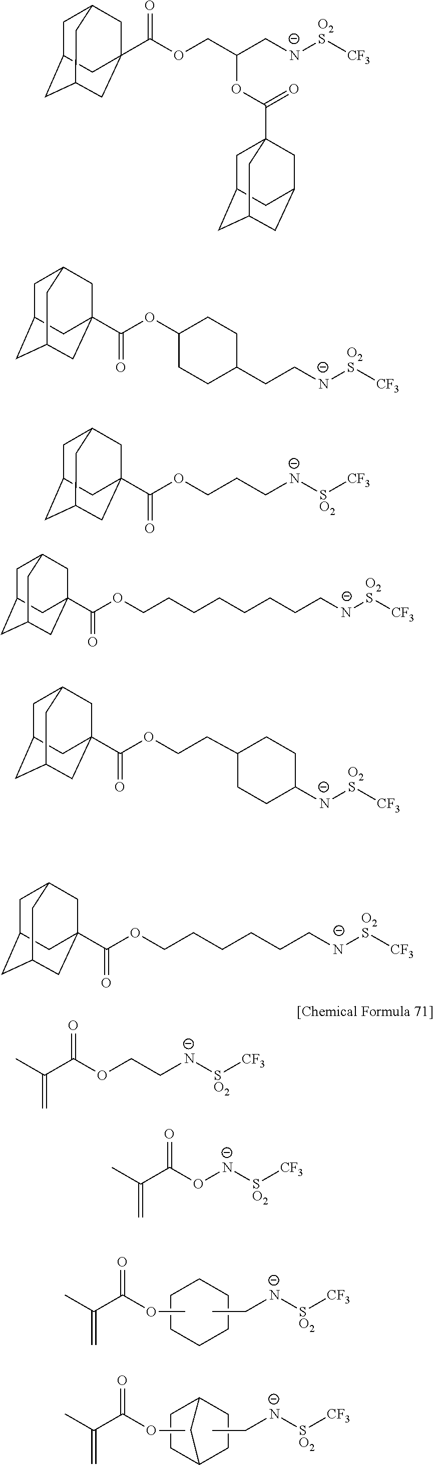

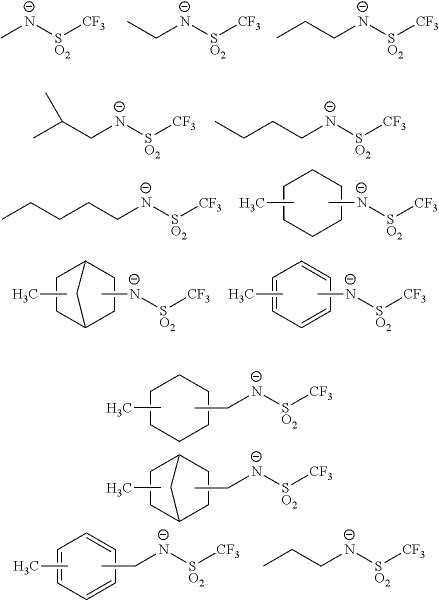

### Cation Moiety

In Formula (d1-3), M^{m+} represents an m-valent organic cation and is the same as M^{m+} in Formula (d1-1).

The component (d1-3) may be used alone or in combination of two or more kinds thereof.

As the component (D1), only one of the above-described components (d1-1) to (d1-3) or a combination of two or more kinds thereof may be used.

In a case where the resist composition contains the component (D1), the content of the component (D1) in the resist composition is preferably 0.5 to 15 parts by mass, more preferably 1 to 10 parts by mass, and still more preferably 2 to 8 parts by mass with respect to 100 parts by mass of the component (A).

In a case where the content of the component (D1) is equal to or greater than the preferable lower limits, particularly satisfactory lithography characteristics and a particularly satisfactory resist pattern shape are likely to be obtained. On the contrary, in a case where the content is equal to or less than the upper limits, the sensitivity can be satisfactorily maintained and the throughput is also excellent.

Method of producing component (D1):

The methods of producing the component (d1-1) and the component (d1-2) are not particularly limited, and these components can be produced by known methods.

Further, the method of producing the component (d1-3) is not particularly limited, and the component is produced, for example, by the same method as disclosed in United States Patent Application, Publication No. 2012-0149916.

A compound of the component (D1) is described as an example of the base component (component (D)) that traps an acid generated upon light exposure, but the compound of the component (D1) may be used as the component (B).

For example, in the resist composition of the present embodiment, a compound of the component (D1) may be used as the component (B), and a compound that generates an acid having an acidity lower than the acid generated by the compound of the component (D1) upon light exposure may be used as the component (D). In addition, in the resist composition of the present embodiment, a compound of the component (D1) may be used as the component (B), and the component (D2) described below may be used as the component (D).

### In Regard to Component (D2)

The component (D) may contain a nitrogen-containing organic compound component (hereinafter, referred to as “component (D2)”) which does not correspond to the component (D1) described above.

The component (D2) is not particularly limited as long as the component acts as an acid diffusion control agent and does not correspond to the component (D1), and any known compound may be used. Among these, an aliphatic amine is preferable, and particularly a secondary aliphatic amine and a tertiary aliphatic amine are more preferable.

The aliphatic amine is an amine containing one or more aliphatic groups, and the aliphatic groups have preferably 1 to 12 carbon atoms.

Examples of these aliphatic amines include amines in which at least one hydrogen atom of ammonia NH₃ has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolamine, triisopropanolamine, di-n-octanolamine, and tri-n-octanolamine. Among these, a trialkylamine having 6 to 30 carbon atoms is still more preferable, and tri-n-pentylamine or tri-n-octylamine is particularly preferable.

Examples of the cyclic amine include a heterocyclic compound having a nitrogen atom as a heteroatom. The heterocyclic compound may be a monocyclic compound (aliphatic monocyclic amine) or a polycyclic compound (aliphatic polycyclic amine).

Specific examples of the aliphatic monocyclic amine include piperidine and piperazine.

The aliphatic polycyclic amine has preferably 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy) ethoxy}ethyl]amine, and triethanolamine triacetate. Among these, triethanolamine triacetate is preferable.

As the component (D2), an aromatic amine may be used.

Examples of aromatic amines include 4-dimethylaminopyridine, pyrrole, indole, pyrazole, imidazole, and derivatives thereof, tribenzylamine, 2,6-diisopropylaniline, N-tert-butoxycarbonylpyrrolidine, and 2,6-di-tert-butylpyridine.

Among these, the component (D2) is preferably an alkylamine and more preferably a trialkylamine having 5 to 10 carbon atoms.

The component (D2) may be used alone or in combination of two or more kinds thereof.

In a case where the resist composition contains the component (D2), the content of the component (D2) in the resist composition is preferably 0.01 to 5 parts by mass, more preferably 0.1 to 5 parts by mass, and still more preferably 0.5 to 5 parts by mass with respect to 100 parts by mass of the component (A).

In a case where the content of the component (D2) is equal to or greater than the preferable lower limits, particularly satisfactory lithography characteristics and a particularly satisfactory resist pattern shape are likely to be obtained. On the contrary, in a case where the content is equal to or less than the upper limits, the sensitivity can be satisfactorily maintained and the throughput is also excellent.

<<At Least One Compound (E) Selected from Group Consisting of Organic Carboxylic Acids, and Phosphorus Oxo Acids and Derivatives Thereof>>

For the purpose of preventing any deterioration of sensitivity and improving the resist pattern shape and the post light exposure temporal stability, the resist composition of the present embodiment may contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

In the resist composition of the present embodiment, the component (E) may be used alone or in combination of two or more kinds thereof.

In a case where the resist composition contains the component (E), the content of the component (E) is preferably 0.01 to 5 parts by mass and more preferably 0.05 to 3 parts by mass with respect to 100 parts by mass of the component (A). In a case where the content of the component (E) is set to be in the above-described ranges, the lithography characteristics are further improved.

### <<Fluorine Additive Component (F)>>

The resist composition of the present embodiment may contain a fluorine additive component (hereinafter, referred to as “component (F)”) as a hydrophobic resin.

The component (F) is used to impart water repellency to the resist film and used as a resin different from the component (A), and thus the lithography characteristics are improved.

As the component (F), for example, the fluorine-containing polymer compounds described in Japanese Unexamined Patent Application, First Publication Nos. 2010-002870, 2010-032994, 2010-277043, 2011-13569, and 2011-128226 can be used.

More specific examples of the component (F) include a polymer having a constitutional unit (f1) represented by General Formula (f1-1). As the polymer, a polymer (homopolymer) consisting of only the constitutional unit (f1) represented by Formula (f1-1); a copolymer of the constitutional unit (f1) and the constitutional unit (a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-1-adamantyl (meth)acrylate is preferable, and a constitutional unit derived from 1-ethyl-1-cyclooctyl (meth)acrylate is more preferable.

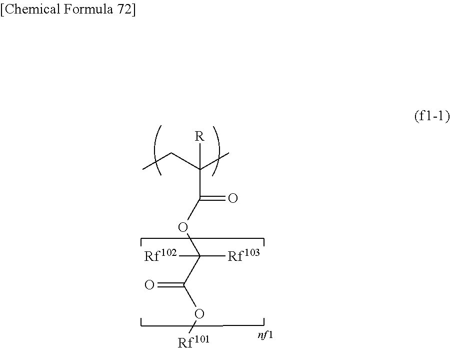

[In the formula, R has the same definition as described above, Rf¹⁰² and Rf¹⁰³ each independently represent a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other. nf¹ represents an integer of 0 to 5, and Rf¹⁰¹ represents an organic group having a fluorine atom.]

In Formula (f1-1), R bonded to the carbon atom at the α-position has the same definition as described above. As R, a hydrogen atom or a methyl group is preferable.

In Formula (f1-1), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R. Among these, a methyl group or an ethyl group is preferable. Specific examples of the halogenated alkyl group having 1 to 5 carbon atoms for Rf¹⁰² and Rf¹⁰³ include groups in which some or all hydrogen atoms of an alkyl group having 1 to 5 carbon atoms have been substituted with halogen atoms. Among these, a fluorine atom is preferable as the halogen atom. Among these, Rf¹⁰² and Rf¹⁰³ represent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ represents an integer of 0 to 5, preferably an integer of 0 to 3, and more preferably 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and has preferably 1 to 20 carbon atoms, more preferably 1 to 15 carbon atoms, and particularly preferably 1 to 10 carbon atoms.

In the hydrocarbon group having a fluorine atom, preferably 25% or more of the hydrogen atoms in the hydrocarbon group are fluorinated, more preferably 50% or more thereof are fluorinated, and particularly preferably 60% or more thereof are fluorinated from the viewpoint of increasing the hydrophobicity of the resist film during immersion light exposure.

Among these, Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

In addition, examples of the component (F) also include a polymer having the constitutional unit (a8). As the polymer, a copolymer of the constitutional unit (a8) and the constitutional unit (a2) is preferable.

The weight-average molecular weight (Mw) (in terms of polystyrene according to gel permeation chromatography) of the component (F) is preferably 1000 to 50000, more preferably 5000 to 40000, and most preferably 10000 to 30000. In a case where the weight-average molecular weight of the component (F) is equal to or less than the upper limits of the above-described ranges, the resist composition exhibits a satisfactory solubility in a solvent for a resist enough to be used as a resist. Meanwhile, in a case where the weight-average molecular weight of the component (F) is equal to or greater than the lower limits of the above-described ranges, water repellency of the resist film is improved.

Further, the dispersity (Mw/Mn) of the component (F) is preferably 1.0 to 5.0, more preferably 1.0 to 3.0, and most preferably 1.0 to 2.5.

In the resist composition of the present embodiment, the component (F) may be used alone or in combination of two or more kinds thereof.

In a case where the resist composition contains the component (F), the content of the component (F) is preferably 0.5 to 10 parts by mass and more preferably 1 to 10 parts by mass with respect to 100 parts by mass of the component (A).

### <<Organic Solvent Component(S)>>

The resist composition of the present embodiment may be produced by dissolving the resist materials in an organic solvent component (hereinafter, referred to as “component (S)”).

The component (S) may be any organic solvent which can dissolve each component to be used to obtain a uniform solution, and an optional organic solvent can be appropriately selected from those which have been known in the related art as solvents of a chemically amplified resist composition and then used.

In the resist composition of the present embodiment, the component (S) may be used alone or as a mixed solvent of two or more kinds thereof. Among these, PGMEA, PGME, γ-butyrolactone, ethyl lactate (EL), or cyclohexanone is preferable.

Further, a mixed solvent obtained by mixing PGMEA with a polar solvent is also preferable as the component (S). The blending ratio (mass ratio) may be appropriately determined in consideration of the compatibility or the like between PGMEA and the polar solvent.

Further, a mixed solvent of γ-butyrolactone and at least one selected from PGMEA and EL is also preferable as the component (S). In this case, as the mixing ratio, the mass ratio between the former and the latter is preferably 70:30 to 95:5.

The amount of the component (S) to be used is not particularly limited and is appropriately set to have a concentration which enables coating a substrate or the like depending on the thickness of the coated film. The component (S) is typically used in an amount such that the solid content concentration of the resist composition is set to be in a range of 0.1% to 20% by mass and preferably in a range of 0.2% to 15% by mass.

After the resist material is dissolved in the component (S), impurities may be removed from the resist composition of the present embodiment using a porous polyimide film, a porous polyamide-imide film, or the like. For example, the resist composition may be filtered using a filter consisting of a porous polyimide film, a filter consisting of a porous polyamide-imide film, a filter consisting of a porous polyimide film and a porous polyamide-imide film, or the like. Examples of the porous polyimide film and the porous polyamide-imide film include those described in Japanese Unexamined Patent Application, First Publication No. 2016-155121.

The resist composition of the present embodiment described above contains the compound (D0) (the component (D0)) represented by General Formula (d0).

The resist composition in the related art has a problem in that the fine resolution is deteriorated due to storage.

In the component (D0), R¹O, which is an alkoxy group, and an iodine atom are bonded to a benzene ring of the anion moiety. In addition, the substituent bonded to the benzene ring of the cation moiety has a fluorine atom.

By the R¹O of the anion moiety and the fluorine atom of the cation moiety of the component (D0), in the resist composition of the present embodiment, the temporal stability during storage is improved and the deterioration of the fine resolution after storage is suppressed. The reason why such an effect is exhibited is not clear, but is presumed as follows.

In the resist composition of the related art, during storage, the cation of the acid generator is decomposed by the anion of the acid diffusion control agent, and thus the fine resolution is deteriorated.

In the resist composition of the present embodiment, during storage, the decomposition of the cation of the acid generator by the anion of the component (D0) is suppressed. As a result, it is presumed that the acid generator is stably stored during storage, and the deterioration of the fine resolution after storage is suppressed.

The iodine atom has a large absorption of EUV having a wavelength of 13.5 nm. Therefore, the component (D0) easily generates secondary electrons during light exposure. In the secondary electrons generated from the component (D0) upon light exposure, in a case where the energy of the secondary electrons is transferred to the component (the base material component (A) or the acid generator component (B)) which generates an acid upon light exposure, the decomposition of the component which generates an acid upon light exposure is promoted. As a result, in a case where a resist pattern is formed using the resist composition of the present embodiment, the sensitivity is increased.

### (Resist Pattern Formation Method)

A resist pattern formation method according to a second aspect of the present invention is a method including a step of forming a resist film on a support using the resist composition according to the first aspect of the present invention described above, a step of exposing the resist film to light, and a step of developing the resist film exposed to light to form a resist pattern.

According to the embodiment of the resist pattern formation method, a resist pattern formation method, which is performed in the following manner is an exemplary example.

First, a support is coated with the resist composition of the embodiment using a spinner or the like, and a bake (post applied bake (PAB)) treatment is performed under a temperature condition of, for example, 80 °C to 150 °C for 40 to 120 seconds and preferably for 60 to 90 seconds to form a resist film.

Next, the selective light exposure is performed on the resist film by, for example, light exposure through a mask (mask pattern) having a predetermined pattern formed thereon using a light exposure apparatus such as an electron beam lithography apparatus or an ArF light exposure apparatus, or direct irradiation with an electron beam for drawing without using a mask pattern, and a bake treatment (post-light exposure bake (PEB)) is carried out, for example, under a temperature condition of 80 °C to 150 °C for 40 to 120 seconds and preferably for 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse solution containing an organic solvent is preferable in a case of the solvent developing process.

In a case of the solvent developing process, after the developing treatment or the rinse treatment, the developing solution or the rinse solution attached onto the pattern may be removed by a treatment using a supercritical fluid.

After the developing treatment or the rinse treatment, drying is conducted. As desired, a bake treatment (post bake) may be conducted after the developing treatment.

The support is not particularly limited and a known support of the related art can be used, and examples thereof include a substrate for an electronic component and a substrate on which a predetermined wiring pattern has been formed. More specific examples thereof include a metal substrate such as a silicon wafer, copper, chromium, iron, or aluminum; and a glass substrate. As the materials of the wiring pattern, copper, aluminum, nickel, or gold can be used.

The wavelength to be used for the light exposure is not particularly limited, and the light exposure can be carried out using radiation such as an ArF excimer laser, a KrF excimer laser, an F2 excimer laser, extreme ultraviolet rays (EUV), vacuum ultraviolet rays (VUV), electron beams (EB), X-rays, or soft X-rays, and it is preferable to carry out the light exposure using EUV or EB.

The method of exposing the resist film to light may be general light exposure (dry light exposure) conducted in air or an inert gas such as nitrogen, or liquid immersion light exposure (liquid immersion lithography).

The liquid immersion light exposure is a light exposure method in which the region between the resist film and the lens at the lowermost position of the light exposure apparatus is filled with a solvent (liquid immersion medium) in advance that has a refractive index greater than the refractive index of air, and the light exposure (immersion light exposure) is conducted in this state.

As the liquid immersion medium, a solvent having a refractive index greater than the refractive index of air but less than the refractive index of the resist film to be exposed to light is preferable, and examples thereof include water, a fluorine-based inert liquid, a silicon-based solvent, and a hydrocarbon-based solvent.

As the liquid immersion medium, water is preferably used.

Examples of the alkali developing solution used for the developing treatment in the alkali developing process include a 0.1% to 10% by mass tetramethylammonium hydroxide (TMAH) aqueous solution.

The organic solvent contained in the organic developing solution used for the developing treatment in the solvent developing process may be any solvent that is capable of dissolving the component (A) (the component (A) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butanoate, methyl 2-hydroxyisobutyrate, isoamyl acetate, isobutyl isobutyrate, and butyl propionate.

Examples of the nitrile-based solvent include acetonitrile, propionitrile, valeronitrile, and butyronitrile.

Known additives can be blended into the organic developing solution as necessary. Examples of the additive include a surfactant.

The developing treatment can be performed according to a known developing method, and examples thereof include a method of immersing a support in a developing solution for a certain time (dip method), a method of raising a developing solution on the surface of a support using the surface tension and maintaining the state for a certain time (puddle method), a method of spraying a developing solution to the surface of a support (spray method), and a method of continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, for example, a solvent that is difficult to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used.

These organic solvents may be used alone or in combination of two or more kinds thereof. Further, an organic solvent other than the above-described solvents and water may be mixed and used.

The rinse treatment carried out using a rinse solution (washing treatment) can be performed according to a known rinse method. Examples of the method of performing the rinse treatment include a method of continuously ejecting a rinse solution onto a support rotating at a certain rate (rotary coating method), a method of immersing a support in a rinse solution for a certain time (dip method), and a method of spraying a rinse solution to the surface of a support (spray method).

Various materials that are used in the resist composition of the above-described embodiment and the resist pattern formation method of the above-described embodiment (for example, a resist solvent, a developing solution, a rinse solution, a composition for forming an antireflection film, and a composition for forming a top coat) preferably do not contain impurities such as a metal, a metal salt containing halogen, an acid, an alkali, and a component containing a sulfur atom or phosphorus atom.

Here, examples of the impurities containing metal atoms include Na, K, Ca, Fe, Cu, Mn, Mg, Al, Cr, Ni, Zn, Ag, Sn, Pb, Li, and salts thereof. The content of the impurities contained in these materials is preferably 200 ppb or less, more preferably 1 ppb or less, still more preferably 100 parts per trillion (ppt) or less, particularly preferably 10 ppt or less, and most preferably substantially zero (equal to or less than the detection limit of the measuring device).

### (Compound)

The compound of the present embodiment is represented by General Formula (d0).

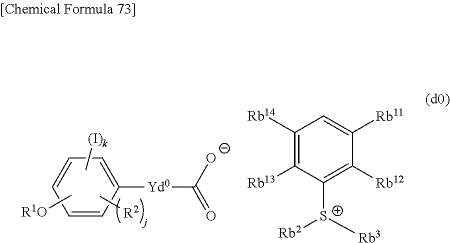

[In the formula, R¹ represents a saturated hydrocarbon group. I represents an iodine atom. R² represents a substituent. Yd⁰ represents a divalent linking group or a single bond. j represents an integer of 0 to 3. k represents an integer of 1 to 4. 1≤j+k≤4 is satisfied. Rb¹¹, Rb¹², Rb¹³, and Rb¹⁴ each independently represent a fluorine atom, a fluorinated alkyl group, or a hydrogen atom. At least one of Rb¹¹, Rb¹², Rb¹³, or Rb¹⁴ is a fluorine atom or a fluorinated alkyl group. Rb² and Rb³ each independently represent a hydrocarbon group which may have a substituent, or Rb² and Rb³ are bonded to each other to form a ring together with a sulfur atom in the formula.]

The compound of the present embodiment is the same as the component (D0) of the resist composition according to the first aspect.

The compound of the present embodiment is useful as an acid diffusion control agent used in the resist composition.

### (Acid Diffusion Control Agent)

The acid diffusion control agent of the present embodiment contains the compound (d0) represented by Formula (d0) in the above-described embodiment.

### (Method of Producing Compound)

The method of producing a compound of the present embodiment is not particularly limited, but may include, for example, the following Reaction 1 and Reaction 2.

In Reaction 1, for example, a compound represented by General Formula (d0-preA0) is reacted with a reaction solvent containing a base compound represented by General Formula (bs0) to obtain a compound represented by General Formula (d0-preB0).

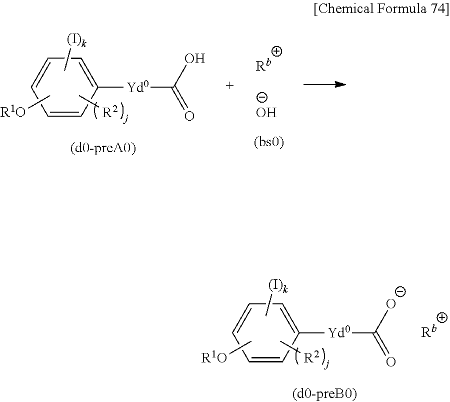

[In the formula, R¹ represents a saturated hydrocarbon group. I represents an iodine atom. R² represents a substituent. Yd⁰ represents a divalent linking group or a single bond. j represents an integer of 0 to 3. k represents an integer of 1 to 4. 1≤j+k≤4 is satisfied. R^{b+} represents an organic cation or an inorganic cation.]

R¹, R², Yd⁰, j, and k in Formula (d0-preA0) and Formula (d0-preB0) are the same as R¹, R², Yd⁰, j, and k in Formula (d0).

Examples of the base compound represented by General Formula (bs0) include tetramethylammonium hydroxide (TMAH).

Examples of R^{b+} include ammonium cations such as a tetramethylammonium cation, a tetrabutylammonium cation, and a triethylammonium cation, and a pyridinium cation.

Examples of the reaction solvent include water, dichloromethane, acetonitrile, and chloroform.

In Reaction 1, the reaction temperature is, for example, 0 °C to 100 °C, and the reaction time is, for example, 10 minutes or more and 24 hours or less.

Next, in Reaction 2, for example, a compound represented by Formula (d0-preB0) and a compound represented by General Formula (d0-s0) are reacted to obtain a compound represented by General Formula (d0).

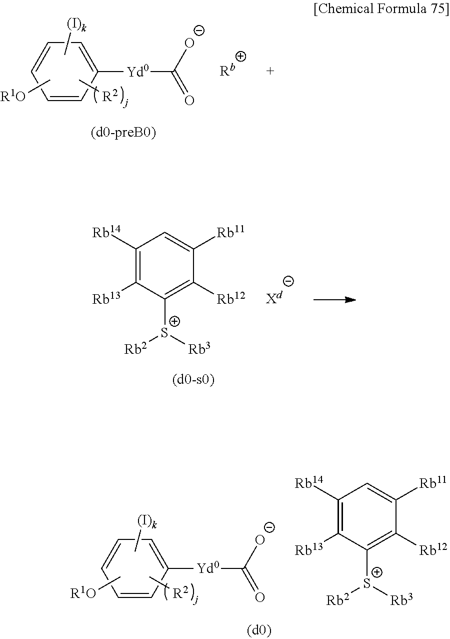

[In the formula, R¹ represents a saturated hydrocarbon group. I represents an iodine atom. R² represents a substituent. Yd⁰ represents a divalent linking group or a single bond. j represents an integer of 0 to 3. k represents an integer of 1 to 4. 1≤j+k≤4 is satisfied. Rb¹¹, Rb¹², Rb¹³, and Rb¹⁴ each independently represent a fluorine atom, a fluorinated alkyl group, or a hydrogen atom. At least one of Rb¹¹, Rb¹², Rb¹³, or Rb¹⁴ is a fluorine atom or a fluorinated alkyl group. Rb² and Rb³ each independently represent a hydrocarbon group which may have a substituent, or Rb² and Rb³ are bonded to each other to form a ring together with the sulfur atom in the formula. R^{b+} represents an organic cation or an inorganic cation. Xd-represents a halogen anion.]

Rb², Rb³, Rb¹¹, Rb¹², Rb¹³, and Rb¹⁴ in Formula (d0-s0) are the same as Rb², Rb³, Rb¹¹, Rb¹², Rb¹³, and Rb¹⁴ in Formula (d0).

X^{d-} in Formula (d0-s0) represents preferably a chloride ion and a bromide ion, and more preferably a chloride ion.

Examples of the reaction solvent in Reaction 2 include the same reaction solvents which can be used in Reaction 1.

In Reaction 2, the reaction temperature is, for example, 0 °C to 100 °C, and the reaction time is, for example, 10 minutes or more and 24 hours or less.

After completion of the salt exchange reaction, the compound (d0) in the reaction solution may be isolated and purified. A known method in the related art can be used for isolation and purification, and for example, concentration, solvent extraction, distillation, crystallization, recrystallization, or chromatography can be appropriately combined and used.

The structure of the compound (d0) obtained as described above can be identified by typical organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), an elemental analysis method, and an X-ray crystal diffraction method.

### EXAMPLES

Hereinafter, the present invention will be described in more detail based on Examples, but the present invention is not limited to these Examples.

### Production Example of Compound

### Production Example 1: Production of Compound (D0-1)

20.0 g of the compound (D0-preA1) and 90.3 g of a 5% aqueous solution of tetramethylammonium hydroxide (BS1) were placed in a three-neck flask, and the mixture was stirred at room temperature for 3 hours to obtain an aqueous solution of the compound (D0-preB1).

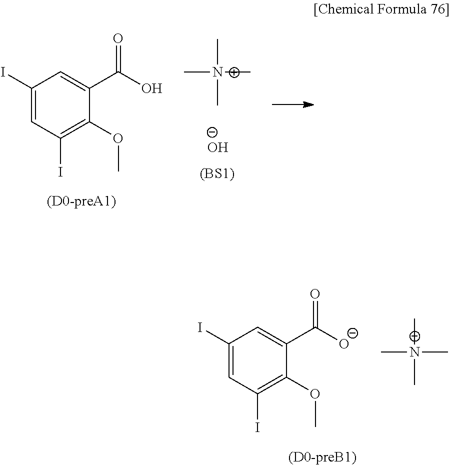

Next, 18.2 g of (D0-S1) and 200 g of dichloromethane were placed in a three-neck flask, and the aqueous solution of the compound (D0-preB1) was stirred for 3 hours. The organic layer was washed with ion exchange water and recovered. The organic layer was concentrated by a rotary evaporator, thereby obtaining 26.9 g of a compound (D0-1).

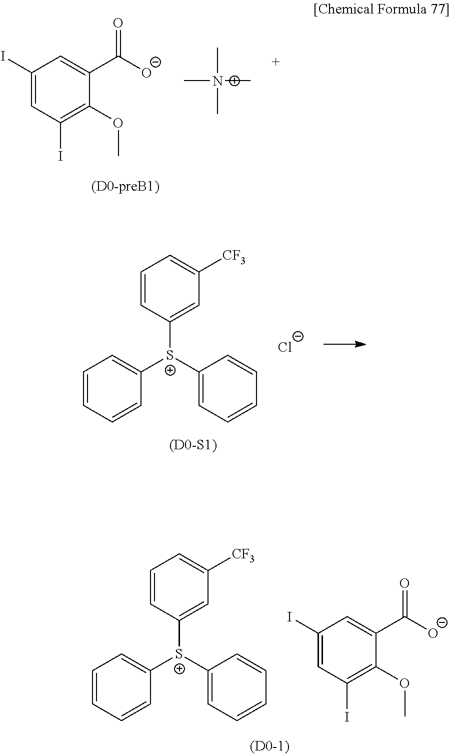

The ¹H NMR measurement results of the compound (D0-1) are shown below.

¹H NMR (400 MHz, DMSO-d6) δ (ppm)=3.83 (3H, s), 7.43 (14H, m), 8.18 (1H, d), 8.30 (1H, d)

### Production Examples 2 to 10: Production of Compounds (D0-2) to (D0-10)

Each of compounds (D0-2) to (D0-10) was obtained in the same manner as in “Production Example 1: production of compound (D0-1)” described above, except that the combinations of the following compounds (D0-preA2) to (D0-preA4) and the following compounds (D0-s2) to (D0-s7) were changed.

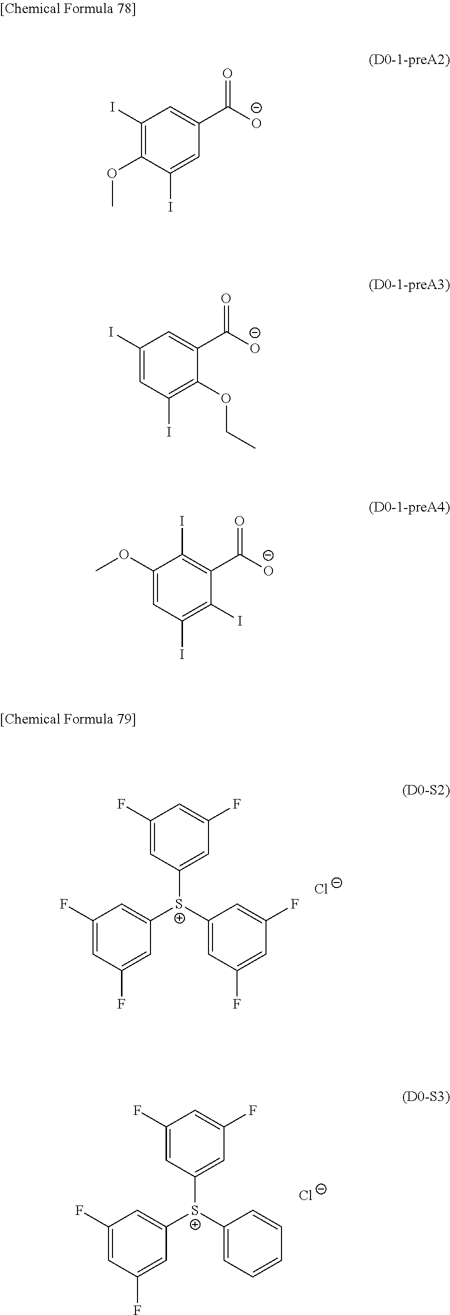

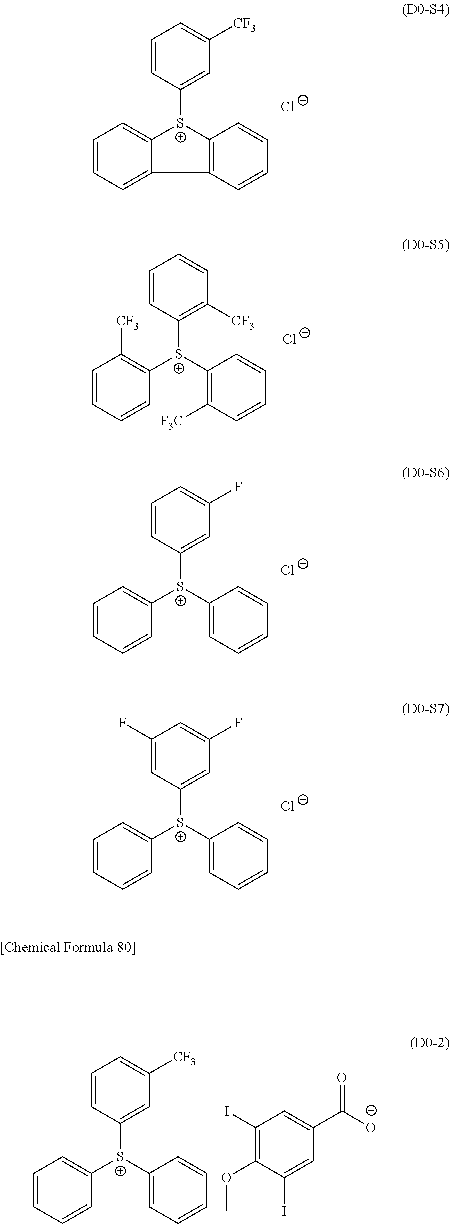

The ¹H NMR measurement results of the compound (D0-2) are shown below.

¹H NMR (400 MHz, DMSO-d6) δ (ppm)=3.83 (3H, s), 7.43 (14H, m), 8.18 (2H, s)

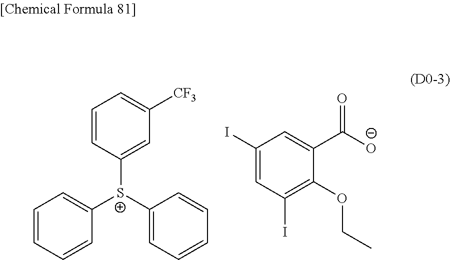 [SMILES: FC(F)(F)c1cccc([S+](c2ccccc2)c2ccccc2)c1 ; CCOc1c(I)cc(I)cc1C(=O)[O-]]

The ¹H NMR measurement results of the compound (D0-3) are shown below.

¹H NMR (400 MHz, DMSO-d6) δ (ppm)=1.34 (3H, t), 4.07 (q. 2H), 7.43 (14H, m), 8.18 (1H, d), 8.30 (1H, d)

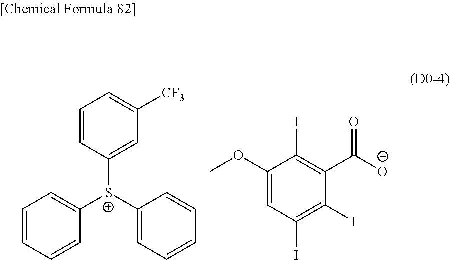 [SMILES: FC(F)(F)c1cccc([S+](c2ccccc2)c2ccccc2)c1 ; COc1cc(I)c(I)c(C(=O)[O-])c1I]

The ¹H NMR measurement results of the compound (D0-4) are shown below.

¹H NMR (400 MHz, DMSO-d6) δ (ppm)=3.93 (3H, s), 7.16 (2H, s), 7.43 (14H, m)

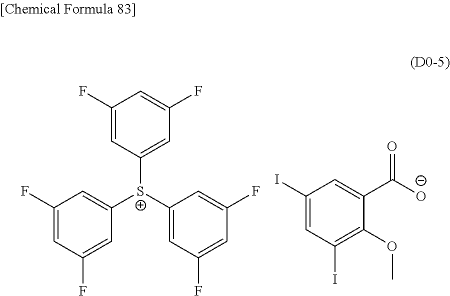 [SMILES: Fc1cc(F)cc([S+](c2cc(F)cc(F)c2)c2cc(F)cc(F)c2)c1 ; COc1c(I)cc(I)cc1C(=O)[O-]]

The ¹H NMR measurement results of the compound (D0-5) are shown below.

¹H NMR (400 MHz, DMSO-d6) δ (ppm)=3.83 (3H, s), 6.77 (9H, m), 8.18 (1H, 5 d), 8.30 (1H, d)

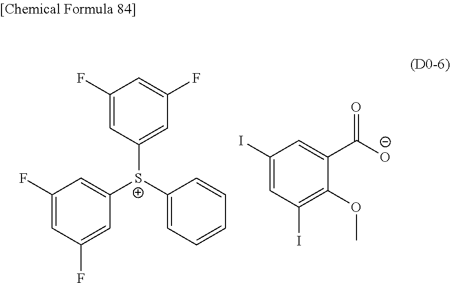 [SMILES: Fc1cc(F)cc([S+](c2ccccc2)c2cc(F)cc(F)c2)c1 ; COc1c(I)cc(I)cc1C(=O)[O-]]

The ¹H NMR measurement results of the compound (D0-6) are shown below.

¹H NMR (400 MHz, DMSO-d6) δ (ppm)=3.83 (3H, s), 6.77 (6H, m), 7.34 (5H, m), 8.18 (1H, d), 8.30 (1H, d)

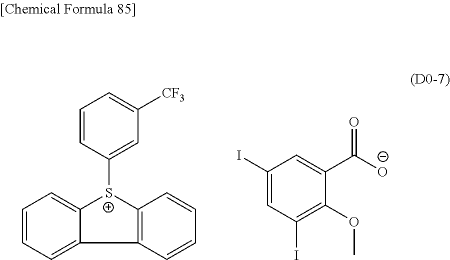 [SMILES: FC(F)(F)c1cccc(-[s+]2c3ccccc3c3ccccc32)c1 ; COc1c(I)cc(I)cc1C(=O)[O-]]

The ¹H NMR measurement results of the compound (D0-7) are shown below.

¹H NMR (400 MHz, DMSO-d6) δ (ppm)=3.83 (3H, s), 7.30 (2H, m), 7.48 (8H, m), 7.77 (2H, m), 8.18 (1H, d), 8.30 (1H, d)

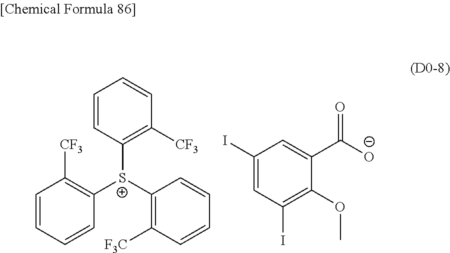 [SMILES: FC(F)(F)c1ccccc1[S+](c1ccccc1C(F)(F)F)c1ccccc1C(F)(F)F ; COc1c(I)cc(I)cc1C(=O)[O-]]

The ¹H NMR measurement results of the compound (D0-8) are shown below.

¹H NMR (400 MHz, DMSO-d6) δ (ppm)=3.83 (3H, s), 7.24 (6H, m), 7.35 (3H, m), 7.47 (3H, m), 8.18 (1H, d), 8.30 (1H, d)

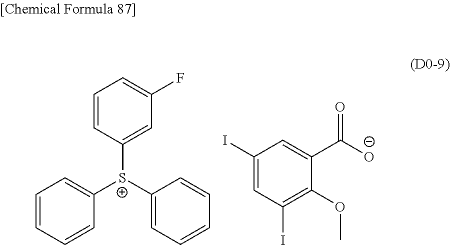 [SMILES: Fc1cccc([S+](c2ccccc2)c2ccccc2)c1 ; COc1c(I)cc(I)cc1C(=O)[O-]]

The ¹H NMR measurement results of the compound (D0-9) are shown below.

¹H NMR (400 MHz, DMSO-d6) δ (ppm)=3.83 (3H, s), 7.09 (3H, m), 7.35 (11H, m), 8.18 (1H, d), 8.30 (1H, d)

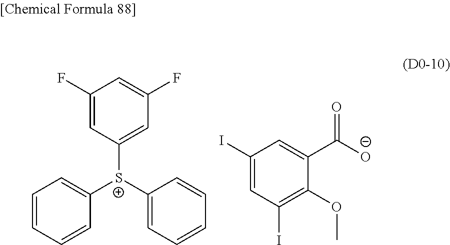 [SMILES: Fc1cc(F)cc([S+](c2ccccc2)c2ccccc2)c1 ; COc1c(I)cc(I)cc1C(=O)[O-]]

The ¹H NMR measurement results of the compound (D0-10) are shown below.

¹H NMR (400 MHz, DMSO-d6) δ (ppm)=3.83 (3H, s), 6.78 (3H, m), 7.35 (10H, m), 8.18 (1H, d), 8.30 (1H, d)

### <Preparation of Resist Composition>

### Examples 1 to 13 and Comparative Examples 1 to 5

Each of the components shown in Table 1 was mixed and dissolved to prepare a resist composition of each Example.

TABLE 1

| | Component | Component | Component | Component |
|---|---|---|---|---|
| | (A) | (B) | (D) | (S) |
| Example 1 | (A)-1 | (B)-1 | (D0)-1 | (S)-1 |
| | [100] | [16.2] | [9.2] | [8000] |
| Example 2 | (A)-1 | (B)-1 | (D0)-2 | (S)-1 |
| | [100] | [16.2] | [9.2] | [8000] |
| Example 3 | (A)-1 | (B)-1 | (D0)-3 | (S)-1 |
| | [100] | [16.2] | [9.3] | [8000] |
| Example 4 | (A)-1 | (B)-1 | (D0)-4 | (S)-1 |
| | [100] | [16.2] | [10.7] | [8000] |
| Example 5 | (A)-1 | (B)-1 | (D0)-5 | (S)-1 |
| | [100] | [16.2] | [9.7] | [8000] |
| Example 6 | (A)-1 | (B)-1 | (D0)-6 | (S)-1 |
| | [100] | [16.2] | [9.2] | [8000] |
| Example 7 | (A)-1 | (B)-1 | (D0)-7 | (S)-1 |
| | [100] | [16.2] | [9.1] | [8000] |
| Example 8 | (A)-1 | (B)-1 | (D0)-8 | (S)-1 |
| | [100] | [16.2] | [10.9] | [8000] |
| Example 9 | (A)-1 | (B)-1 | (D0)-9 | (S)-1 |
| | [100] | [16.2] | [8.5] | [8000] |
| Example 10 | (A)-1 | (B)-1 | (D0)-10 | (S)-1 |
| | [100] | [16.2] | [8.8] | [8000] |
| Example 11 | (A)-2 | (B)-1 | (D0)-1 | (S)-1 |
| | [100] | [16.2] | [9.2] | [8000] |
| Example 12 | (A)-3 | (B)-1 | (D0)-1 | (S)-1 |
| | [100] | [16.2] | [9.2] | [8000] |
| Example 13 | (A)-1 | (B)-2 | (D0)-1 | (S)-1 |
| | [100] | [20.0] | [9.2] | [8000] |
| Comparative | (A)-1 | (B)-1 | (D1)-1 | (S)-1 |
| Example 1 | [100] | [16.2] | [8.3] | [8000] |
| Comparative | (A)-1 | (B)-1 | (D1)-2 | (S)-1 |
| Example 2 | [100] | [16.2] | [8.5] | [8000] |
| Comparative | (A)-1 | (B)-1 | (D1)-3 | (S)-1 |
| Example 3 | [100] | [16.2] | [8.3] | [8000] |
| Comparative | (A)-1 | (B)-1 | (D1)-4 | (S)-1 |
| Example 4 | [100] | [16.2] | [9.0] | [8000] |
| Comparative | (A)-1 | (B)-1 | (D1)-5 | (S)-1 |
| Example 5 | [100] | [16.2] | [9.1] | [8000] |

In Table 1, each abbreviation has the following meaning. The numerical values in the brackets are blending amounts (parts by mass).

### (A)-1: Polymer Compound Represented by Chemical Formula (A1)-1

The weight-average molecular weight (Mw) of the polymer compound (A1)-1 in terms of standard polystyrene, determined by GPC measurement, was 6900, and the polydispersity (Mw/Mn) thereof was 1.74. The copolymerization compositional ratio (the ratio (molar ratio) between constitutional units in the structural formula) determined by ¹³C-NMR was l/m=60/40.

### (A)-2: Polymer Compound Represented by Chemical Formula (A1)-2

The weight-average molecular weight (Mw) of the polymer compound (A1)-2 in terms of standard polystyrene, determined by GPC measurement, was 7200, and the polydispersity (Mw/Mn) thereof was 1.72. The copolymerization compositional ratio (the ratio (molar ratio) between constitutional units in the structural formula) determined by ¹³C-NMR was l/m=60/40.

(A)-3: polymer compound represented by Chemical Formula (A1)-3

The weight-average molecular weight (Mw) of the polymer compound (A1)-3 in terms of standard polystyrene, determined by GPC measurement, was 7100, and the polydispersity (Mw/Mn) thereof was 1.72. The copolymerization compositional ratio (the ratio (molar ratio) between constitutional units in the structural formula) determined by ¹³C-NMR was l/m=60/40.

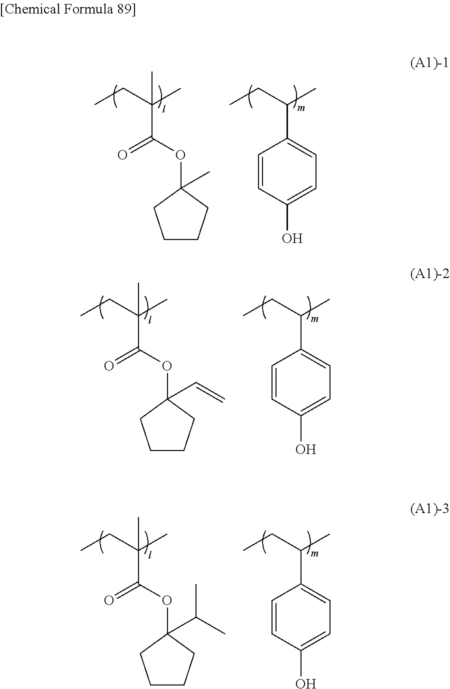

(B)-1: acid generator consisting of compound (B1-1) shown below
(B)-2: acid generator consisting of compound (B1-2) shown below

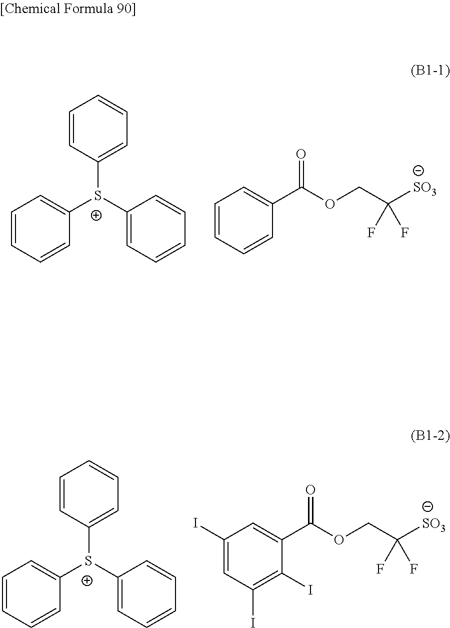

(D0)-1: acid diffusion control agent consisting of compound (D0-1)
(D0)-2: acid diffusion control agent consisting of compound (D0-2)
(D0)-3: acid diffusion control agent consisting of compound (D0-3)
(D0)-4: acid diffusion control agent consisting of compound (D0-4)
(D0)-5: acid diffusion control agent consisting of compound (D0-5)
(D0)-6: acid diffusion control agent consisting of compound (D0-6)
(D0)-7: acid diffusion control agent consisting of compound (D0-7)
(D0)-8: acid diffusion control agent consisting of compound (D0-8)
(D0)-9: acid diffusion control agent consisting of compound (D0-9)
(D0)-10: acid diffusion control agent consisting of compound (D0-10)
(D1)-1: acid diffusion control agent consisting of compound (D1-1) shown below
(D1)-2: acid diffusion control agent consisting of compound (D1-2) shown below
(D1)-3: acid diffusion control agent consisting of compound (D1-3) shown below
(D1)-4: acid diffusion control agent consisting of compound (D1-4) shown below
(D1)-5: acid diffusion control agent consisting of compound (D1-5) shown below

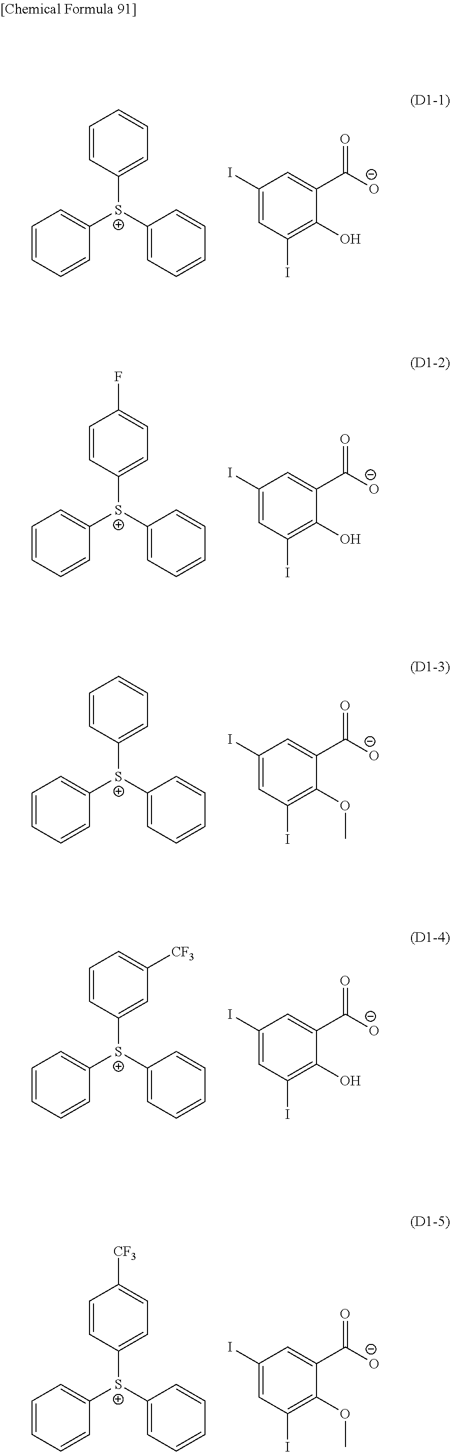

(S)-1: mixed solvent of propylene glycol monomethyl ether acetate/propylene glycol monomethyl ether=60/40 (mass ratio)

### <Resist Pattern Formation>

The resist composition of each Example was allowed to stand and stored at −20 °C for 3 weeks.

An 8-inch silicon substrate which had been subjected to a hexamethyldisilazane (HMDS) treatment was coated with the resist composition of each Example, which was stored at −20 °C, using a spinner, and subjected to a pre-bake (PAB) treatment on a hot plate at a temperature of 110 °C for 60 seconds so that the 8-inch silicon substrate was dried, thereby forming a resist film having a film thickness of 50 nm.

Next, drawing (light exposure) was carried out on the resist film by using an electron beam lithography apparatus JEOL-JBX-9300FS (manufactured by JEOL Ltd.), with the target size being set to a 1:1 line and space pattern (hereinafter, referred to as an “LS pattern”) of a line width of 50 nm, at an acceleration voltage of 100 kV. Thereafter, a post-light exposure bake (PEB) treatment was performed thereon at 100 °C for 60 seconds.

Next, alkali development was performed at 23 °C for 60 seconds using a 2.38% by mass tetramethylammonium hydroxide (TMAH) aqueous solution “NMD-3” (trade name, manufactured by TOKYO OHKA KOGYO CO., LTD.). Thereafter, water rinsing was performed for 15 seconds using pure water. As a result, a 1:1 LS pattern having a line width of 50 nm was formed.

In the above-described resist pattern formation method, a light exposure amount at which an LS pattern having a target size was formed was determined as an optimum light exposure amount Eop (μC/cm²).

In addition, the resist composition of each Example was allowed to stand and stored at 40 °C for 3 weeks.

A resist pattern was formed by the same method as in a case where the resist composition of each Example stored at −20 °C was used, using the resist composition of each Example stored at 40 °C. The light exposure amount was set to the optimum light exposure amount Eop determined above.

### [Evaluation of Temporal Change in Line Pattern Dimension]

In a case where the resist composition stored at −20 °C was used, the line pattern dimension of the obtained LS pattern was defined as CD1.

In a case where the resist composition stored at 40 °C was used, the line pattern dimension of the obtained LS pattern was defined as CD2.

The amount of change in the line pattern dimension over time (ACD) was calculated as CD1-CD2, that is, a difference between CD1 and CD2. The results are listed in the columns of “ΔCD (nm)” in Table 2.

The smaller the value of ΔCD, the more excellent the temporal stability of the resist composition.

### [Evaluation of Temporal Change in Linewise Roughness (LWR)]

In a case where the resist composition stored at −20 °C was used, 3σ of the obtained LS pattern was defined as LWR1.

In a case where the resist composition stored at 40 °C was used, 3σ of the obtained LS pattern was defined as LWR2.

The term “30” means a triple value (30) (unit: nm) of the standard deviation (σ) determined from measurement results obtained by measuring 400 line positions in the longitudinal direction of the line with a scanning electron microscope (acceleration voltage: 800V, trade name: S-9380, manufactured by Hitachi High-Tech Corporation).

The amount of change in the LWR over time (ALWR) was defined as LWR1-LWR2, that is, a difference between LWR1 and LWR2. The results are listed in the columns of “ΔLWR (nm)” in Table 2.

The smaller the value of ΔLWR, the more excellent the temporal stability of the resist composition.

TABLE 2

| | ΔLWR | ΔCD |
|---|---|---|
| | (nm) | (nm) |
| Example 1 | 0.6 | 0.2 |
| Example 2 | 0.6 | 0.2 |
| Example 3 | 0.7 | 0.3 |
| Example 4 | 0.5 | 0.2 |
| Example 5 | 0.5 | 0.2 |
| Example 6 | 0.6 | 0.4 |
| Example 7 | 0.4 | 0.2 |
| Example 8 | 0.5 | 0.2 |
| Example 9 | 1.4 | 0.8 |
| Example 10 | 0.8 | 0.5 |
| Example 11 | 0.6 | 0.3 |
| Example 12 | 0.6 | 0.2 |
| Example 13 | 0.6 | 0.2 |
| Comparative | 5.8 | 2.6 |
| Example 1 | | |
| Comparative | 5.2 | 2.1 |
| Example 2 | | |
| Comparative | 4.2 | 1.9 |
| Example 3 | | |
| Comparative | 5.7 | 2.6 |
| Example 4 | | |
| Comparative | 4.0 | 1.7 |
| Example 5 | | |

From the results shown in Table 2, in the resist compositions of Examples 1 to 13 to which the present invention is applied, the anion moiety of the compound (D0) has an alkoxy group, and in the benzene ring of the cation moiety of the compound (D0), a fluorine atom or a trifluoromethyl group is bonded to the sulfur atom at the ortho position or the meta position. The resist compositions of Examples 1 to 13 have ΔCD of 1.5 nm or less and ΔLWR of 2 nm or less.

On the other hand, in all of the resist compositions of Comparative Examples 1 to 5 which are outside the scope of the present invention, ΔCD is more than 1.5 nm, and ΔLWR is more than 2 nm.

In Example 10 in which the cation of the compound (D0) has two fluorine atoms, both ΔCD and ΔLWR are further reduced as compared with Example 9 in which the cation of the compound (D0) has one fluorine atom.

In Example 6 in which the cation of the compound (D0) has four fluorine atoms, both ΔCD and ΔLWR are further reduced as compared with Example 10 in which the cation of the compound (D0) has two fluorine atoms.

In Example 5 in which the cation of the compound (D0) has six fluorine atoms, both ΔCD and ΔLWR are further reduced as compared with Example 6 in which the cation of the compound (D0) has four fluorine atoms.

In Example 7 in which Rb² and Rb³ are bonded to each other to form a ring together with a sulfur atom in the cation of the compound (D0), both ΔCD and ΔLWR are further reduced as compared with Example 1 in which Rb² and Rb³ are not bonded to each other to form a ring together with a sulfur atom in the cation of the compound (D0).

What is claimed is:

1. A resist composition which generates an acid upon light exposure and whose solubility in a developing solution is changed by an action of the acid, the resist composition comprising:
a base material component (A) whose solubility in a developing solution is changed by the action of the acid; and
a compound (D0) represented by General Formula (d0), 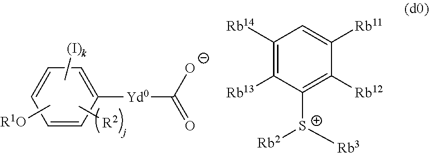
wherein R¹ represents a saturated hydrocarbon group, I represents an iodine atom, R² represents a substituent, Yd⁰ represents a divalent linking group or a single bond, j represents an integer of 0 to 3, k represents an integer of 1 to 4, 1≤j+k≤4 is satisfied, Rb¹¹, Rb¹², Rb¹³, and Rb¹⁴ each independently represent a fluorine atom, a fluorinated alkyl group, or a hydrogen atom, at least one of Rb¹¹, Rb¹², Rb¹³, or Rb¹⁴ is a fluorine atom or a fluorinated alkyl group, and Rb² and Rb³ each independently represents a hydrocarbon group which may have a substituent, or Rb² and Rb³ are bonded to each other to form a ring together with a sulfur atom in the formula.

2. The resist composition according to claim 1, wherein the number of all fluorine atoms included in Rb¹¹, Rb¹², Rb¹³, and Rb¹⁴ in General Formula (d0) is 2 or greater.

3. The resist composition according to claim 1, wherein Rb² and Rb³ in General Formula (d0) each independently represents a phenyl group which may have a substituent.

4. A resist pattern formation method comprising:
forming a resist film on a support using the resist composition according to claim 1;
exposing the resist film to light; and
developing the resist film exposed to light to form a resist pattern.

5. The resist pattern formation method according to claim 4, wherein in exposing the resist film to light, the resist film is exposed to an extreme ultraviolet ray or an electron beam.

6. A compound which is represented by General Formula (d0), 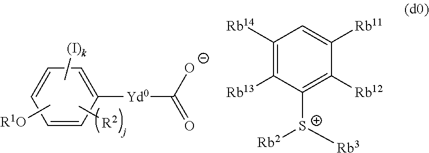
wherein R¹ represents a saturated hydrocarbon group, I represents an iodine atom, R² represents a substituent, Yd⁰ represents a divalent linking group or a single bond, j represents an integer of 0 to 3, k represents an integer of 1 to 4, 1≤j+k≤4 is satisfied, Rb¹¹, Rb¹², Rb¹³, and Rb¹⁴ each independently represents a fluorine atom, a fluorinated alkyl group, or a hydrogen atom, at least one of Rb¹¹, Rb¹², Rb¹³, or Rb¹⁴ is a fluorine atom or a fluorinated alkyl group, and Rb² and Rb³ each independently represents a hydrocarbon group which may have a substituent, or Rb² and Rb³ are bonded to each other to form a ring together with a sulfur atom in the formula.

7. An acid diffusion control agent comprising the compound according to claim 6.
